(19) United States
(12) Patent Application Publication
(10) Pub. No.: US 2026/0345507 A1
(43) Pub. Date: Nov. 5, 2026

(54) COMPOSITIONS AND METHODS FOR DELIVERY OF RNA

(71) Applicant: REJUVENATION TECHNOLOGIES, INC., Mountain View, CA (US)

(72) Inventors: John RAMUNAS, Mountain View, CA (US); Glenn Jeremy MARKOV, Mountain View, CA (US)

(73) Assignee: REJUVENATION TECHNOLOGIES, INC., Mountain View, CA (US)

(21) Appl. No.: 19/106,852

(22) PCT Filed: Aug. 29, 2023
(86) PCT No.: PCT/US2023/073104

§ 371 Date: Feb. 26, 2025

Related U.S. Application Data
(60) Provisional application No. 63/402,195, filed on Aug. 30, 2022.

(51) Int. Cl. A61K 9/51; A61K 9/00; A61K 31/7105

(52) U.S. Cl. CPC A61K 9/5123; A61K 9/0019; A61K 31/7105

(57) ABSTRACT

The present invention relates to lipid nanoparticle (LNP) compositions, and therapeutic polynucleotides, e.g. TERT mRNA, which may be delivered with the LNP compositions in formulations for the prevention or treatment of lung disease and/or fibrosis.

Numerous lung diseases have been identified for which there is no current treatment. Lung diseases, e.g., pulmonary fibrosis, interstitial lung disease, and lung cancer, often induce fibrosis as part of disease progression, which further limits the extent to which patient recovery can occur. Treatment options for lung diseases remain limited. Delivery of polynucleotides to the lung may provide much-needed treatment options to patients with lung diseases; however, the compositions and methods for delivery of polynucleotides are in need of improvement.

Thus, there remains a need in the art for delivery formulations capable of delivering polynucleotides, e.g., mRNAs, to the lung which prevent or treat lung disease and/or lung fibrosis.

The present invention addresses this and other related needs in the art.

### SUMMARY

According to embodiments provided herein, are methods of delivering a polynucleotide to a subject, comprising administering, by intravenous injection, a polynucleotide encapsulated in a lipid nanoparticle (LNP) comprising: (i) a cationic lipid in a molar percentage of between about 20% and about 50%, (ii) a SS-OP or an SS-OP analog at a molar percentage of between about 20% and about 60%, wherein the polynucleotide comprises a synthetic RNA, which upon or after administration of the LNP, the synthetic RNA is translated into a corresponding protein encoded by the synthetic RNA in vivo in the subject.

According to related embodiments, the cationic lipid is (a) SS-OP or an SS-OP analog at a molar percentage of between about 25% and about 60%, wherein the LNP has a lipid nitrogen:polynucleotide phosphate ratio (N/P ratio) of between 10 and 30, or (b) DLin-MC3-DMA, or a DLin-MC3-DMA analog, at a molar percentage of between at or about 30% and at or about 50%, wherein the LNP has an N/P ratio of between 3.5 and 10. Herein, the term “MC3” used by itself means “DLin-MC3-DMA.”

According to one embodiment, the half-life of the lipid DOTAP in vivo in the: lung of the subject is at least 35 hours; liver of the subject is no more than 15 hours; and/or spleen of the subject is no more than 9 hours.

According to related embodiments, the half-life of the lipid DOTAP in vivo in the: lung of the subject is at least 5, 7, 9, 10, 15, 20, 25, 30, 35, 37, 40, 45 or more hours. Also, according to related embodiments, the half-life of the lipid DOTAP in vivo in the liver of the subject is no more than 4, 5, 6, 7, 8, 9, 10, 11, 12, 13, or 15 hours. Also, according to related embodiments, the half-life of the lipid DOTAP in vivo in the spleen of the subject is no more than 4, 5, 6, 7, 8, 9, 10, 11, 12, 13, or 15 hours.

Also according to embodiments provided herein are methods of delivering a polynucleotide to a subject, comprising administering, by intravenous injection, a polynucleotide encapsulated in a lipid nanoparticle (LNP) comprising: (i) a cationic lipid in a molar percentage of between about 20% and about 50%, (ii) a SS-OP or an SS-OP analog at a molar percentage of between about 20% and about 60%, wherein the polynucleotide comprises a synthetic RNA, which upon or after administration of the LNP, the synthetic RNA is translated into a corresponding protein encoded by the synthetic RNA in vivo in the subject, and wherein at or about 6 hours after intravenous infusion of the LNP, the lung: liver ratio of the amount of the protein per mg of tissue is at least 3:1, 4:1, or 5:1. Often, according to related embodiments the lung:liver ratio of the amount of the lipid DOTAP per mg of tissue is at least 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, or 50:1. Also often, according to related embodiments the recited ratio is at or about 3, 4, 5, 6, 7, 8, 9, 10, 11, 12, 24, 48, 72, or 168 hours after intravenous infusion of the LNP encapsulating the mRNA.

Also according to embodiments provided herein are methods of delivering a polynucleotide to a subject, comprising administering, by intravenous injection, a polynucleotide encapsulated in a lipid nanoparticle (LNP) comprising: (i) a cationic lipid in a molar percentage of between about 20% and about 50%, (ii) a SS-OP or an SS-OP analog at a molar percentage of between about 20% and about 60%, wherein the polynucleotide comprises a synthetic RNA, which upon or after administration of the LNP, the synthetic RNA is translated into a corresponding protein encoded by the synthetic RNA in vivo in the subject, and wherein the median telomere length of lung alveolar cells is extended by at least 5%, at least 10%, at least 20%, or at least 50%. Often, according to related embodiments the median telomere length of lung alveolar cells is extended by at least 6%, 7%, 8%, 9%, 11%, 12%, 13%, 14%, 15%, 16%, 17%, 18%, 19%, 25%, 30%, 35%, 40%, 45%, or more. Often, according to related embodiments the median telomere length of lung alveolar cells is extended by at least 50, 100, 200, 300, 400, 500, 1000 base pairs or more.

The synthetic RNA may comprise a sequence encoding a telomerase reverse transcriptase (TERT) protein or part thereof, selected from human TERT (hTERT), mouse TERT (mTERT), or TERT of another mammalian species. According to the presently contemplated embodiments, the mRNA often comprises SEQ ID NO: 1 or a fragment thereof. Also according to the presently contemplated embodiments, the synthetic ribonucleic acid (RNA) encodes telomerase reverse transcriptase (TERT), wherein optionally the TERT mRNA comprises a nucleic acid sequence at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to any one of SEQ ID NOS: 1-5, or SEQ ID NOS: 38-40 of PCT/US22/22642.

Often the TERT synthetic mRNA comprises a 5′ cap structure, wherein the 5′ cap structure is m7(3′OMeG)(5′)ppp(5′)(2′OMeA)pG, IRES, Cap0, Cap1, ARCA, inosine, N1-methylguanosine, 2′fluoro-guanosine, 7-deaza-guanosine, CleanCap™, 8-oxo-guanosine, 2-aminoguanosine, LNA-guanosine, 2-azido-guanosine, Cap2, Cap4, CAP-003, or CAP-225.

Often according to the present methods, the delivery of the polynucleotide is for the prevention and/or treatment of lung disease and/or lung fibrosis in the subject. Also often, according to the present methods the method of delivering the polynucleotide is for the modulation of lung disease and/or lung fibrosis in the subject.

According to often included embodiments, the LNP has a lipid nitrogen:polynucleotide phosphate (N/P) ratio of between 28 and 31. In certain embodiments the N/P ratio is at or about 29.1. Often in such embodiments the ionizable lipid is SS-OP and the LNP has a cationic lipid to ionizable lipid ratio at or about 1.8 and is composed of 5 lipids. In related embodiments, the ionizable lipid is SS-OP and the LNP has a cationic lipid to ionizable lipid ratio at or about 1.8 and is composed of 5 lipids, including DOTAP and DOPC. In related embodiments, the ionizable lipid is SS-OP and the LNP has a cationic lipid to ionizable lipid ratio at or about 1.8 and is composed of 5 lipids, including DMG-PEG2000, DOPC and DOTAP.

According to often included embodiments, the LNP has a N/P ratio of between 11 and 13. In certain embodiments the N/P ratio is at or about 12]. Often in such embodiments the ionizable lipid is SS-OP and the LNP has a cationic lipid to ionizable lipid ratio at or about 0.8 and is composed of 3 or 5 lipids. In related embodiments, the ionizable lipid is SS-OP and the LNP has a cationic lipid to ionizable lipid ratio at or about 0.8 and is composed of 3 or 5 lipids, including DMG-PEG2000 and DOTAP.

According to often included embodiments, the LNP has a N/P ratio of between 4 and 5. In certain embodiments the N/P ratio is at or about 4.5. Often in such embodiments the ionizable lipid is DLin-MC3-DMA and the LNP has a cationic lipid to ionizable lipid ratio at or about 1.5 and is composed of 3 lipids. In related embodiments, the ionizable lipid is DLin-MC3-DMA and the LNP has a cationic lipid to ionizable lipid ratio at or about 1.5 and is composed of 3 lipids, including DMG-PEG2000 and DOTAP.

Also according to methods provided herein, the ionizable lipid is DLin-MC3-DMA and the LNP has a cationic lipid to ionizable lipid ratio at or about 1.5 and is composed of 3 lipids, including DMG-PEG2000 and DOTAP, and N/P ratio is at or about 4.5.

Also according to often included embodiments the LNP is selected from an LNP of Table 8-10.

According to embodiments (FIG. 22A), the LNP has only a single cationic lipid, for example DOTAP.

Also provided herein are methods manufacture of LNPs. In one specific example, the LNPs are manufactured in a manner that forms the LNP prior to addition of nucleic acid. In specific embodiments thereof, such LNPs are 2, 3 or 5 lipids LNPs described herein in Table 8 and the method employs vortex mixing during the addition of the nucleic acid. In certain embodiments the LNP is composed of 2 or 3 lipid LNPs described herein.

These and other embodiments, features, and advantages will become apparent to those skilled in the art when taken with reference to the following more detailed description of various exemplary embodiments of the present disclosure in conjunction with the accompanying drawings.

### BRIEF DESCRIPTION OF THE DRAWINGS

The skilled person in the art will understand that the drawings, described below, are for illustration purposes only.

FIGS. 1A-1D depict the pharmacokinetics of specific lipid nanoparticles (LNPs). Timeline of serum (FIG. 1A), lung (FIG. 1B), and liver (FIG. 1C) levels of the LNPs as determined by measuring one of the lipids, DOTAP. FIG. 1D provides tabular data related to FIGS. 1A-1C.

FIG. 2 depicts the ratio of bioluminescence signal from the indicated organs, produced from luciferase protein translated from a formulation of mRNA in LNPs (“mRNA LNPs”) injected intravenously, at the indicated time points post intravenous injection.

FIG. 3 shows that LNPs formulated with TERT mRNA (“TERT mRNA LNPs”) extend telomeres in lung cells in vivo, where telomere length was measured in lung tissue sections using Q-FISH, and the telomere signal intensity (green/lighter) is proportional to signal length.

FIG. 4 shows that TERT mRNA LNPs extend telomeres in lung cells in vivo, where TERT mRNA LNPs extend median and average telomere length in AT2 cells. At least 50 pro-SPC-positive cell nuclei were quantified per mouse. The error bars are standard error of the mean (SEM) for the telomere length between mice.

FIG. 5 shows that TERT mRNA LNPs extend telomeres in lung cells in vivo, where TERT mRNA LNPs extend median and average telomere length in lung alveolar cells. Telomeres of at least 100 alveolar cells were quantified per mouse. The error bars are standard error of the mean (SEM) for the telomere length between mice.

FIG. 6A shows experimental design and timeline of dosing for data of the present disclosure presented in FIGS. 3-12 and FIGS. 20-21.

FIG. 6B shows experimental design and timeline of dosing for data of the present disclosure presented in FIGS. 13-16.

FIG. 7 shows that TERT mRNA LNPs reduce fibrosis in lung in vivo, where high-density fibrotic foci (yellow/darker) are mapped using picrosirius red staining in lungs in bleomycin-treated mice treated with either control mRNA LNPs (carrying luciferase mRNA) or TERT mRNA LNPs. “No bleo” indicates a lung section from a normal healthy mouse, not treated with bleomycin.

FIG. 8 shows that TERT mRNA LNPs reduce fibrosis in lung in vivo, as measured by quantification of high-density fibrotic foci in Picrosirius Red stained tissue sections.

FIG. 9 shows that TERT mRNA LNPs increase the amount of useful lung volume in vivo, as determined by quantification of the change in normo-aerative lung ratio from baseline using 3D quantification of fibrosis detected by signal intensity in x-ray computed tomography scan data.

FIG. 10 shows that TERT mRNA LNPs improve tissue elastance in vivo, as determined by quantification of measurement of tissue elastance in mice at the end of the study using the Flexivent system.

FIG. 11 shows that TERT mRNA LNPs improve lung function in vivo, as determined by measurement of forced expiratory volume in mice at the end of the study using the Flexivent system.

FIG. 12 shows that TERT mRNA LNPs improve lung function in vivo as determined by measurement of forced vital capacity in mice at the end of the study using the Flexivent system.

FIG. 13 shows that TERT mRNA LNPs improve lung architecture in vivo, where Morpho-Quant Lung software was used to analyze digitized images of tissue sections to quantify alveolar number per unit area.

FIG. 14 shows that TERT mRNA LNPs improve lung architecture in vivo, where Morpho-Quant Lung software was used to analyze digitized images of tissue sections to quantify alveolar diameter.

FIG. 15 shows that TERT mRNA LNPs improve lung architecture in vivo, where Morpho-Quant Lung software was used to analyze digitized images of tissue sections to quantify alveolar circularity.

FIG. 16 shows that TERT mRNA LNPs improve lung architecture in vivo, where representative images of alveoli false colored by Morpho-Quant Lung software during the digitized analysis of alveolar architecture are shown.

FIG. 17 shows pharmacodynamic data of TERT mRNA in vivo following intravenous infusion in a formulation of TERT mRNA LNPs, where in the level of telomerase activity in lung tissue is quantified at the indicated time points following infusion.

FIG. 18 shows an experimental schema for quantifying the effect of TERT mRNA LNPs on human primary epithelial cell colony formation capacity.

FIG. 19 shows that TERT mRNA LNPs increase the colony formation capacity of human primary epithelial cells in the experimental model shown in FIG. 18.

FIG. 20. Depicts levels of P21+senescent lung alveolar cells involving the LNP of Table 3, based on the experimental model shown in FIG. 6A

FIG. 21. is a representative immunohistochemistry image of anti-P21 staining of lung sections, as quantified in FIG. 20.

FIGS. 22A, 22B, 22C and 22D provide lung delivery data obtained using 2 lipid LNP compositions.

FIG. 23 depicts the results of titrating lipid:mRNA ratio in 2 lipid LNP.

FIG. 24 depicts the time course of protein expression after delivery of lung-targeting mRNA-LNP encoding firefly luciferase using the LNPs of Table 3.

FIGS. 25A, 25B and 25C provide lung radiance, encapsulation efficiency, and body weight variance in titrating SS-OP lipid into the ‘DOTAP+PEG’ LNP using LNPs of FIG. 25D.

FIGS. 26A and 26B lung and liver delivery data using the LNP of Table 4.

FIGS. 27A and 27B provide exemplary LNPs and encapsulation efficiency thereof.

FIGS. 27C and 27D provide lung delivery data and body weight variance data in titrating SS-OP lipid into the ‘DOTAP+PEG’ LNP using LNPs of FIG. 27A.

FIGS. 28A and 28B provide comparative fresh versus long term cryopreserved lung transfection and weight data for LNPs of Table 3.

FIGS. 29A and 29B provide comparative fresh versus short term cryopreserved lung transfection and particle size data for LNPs of Table 3.

FIG. 30A provides exemplary 2, 3 and 5 lipid LNPs.

FIGS. 30B and 30C provide lung delivery data and body weight variance data in titrating N/P ratios and comparing 2, 3, and 5 lipid formulations of FIG. 30A.

FIGS. 31A and 31B provide exemplary 3 and 5 lipid LNPs and encapsulation efficiencies thereof.

FIG. 31C provides lung delivery data in titrating N/P ratio (mRNA to lipid ratio) with 3 and 5 lipid formulations 30A.

FIGS. 32A and 32B provide lung transfection data and LNP size and encapsulation efficiency data for LNPS having lipid ratios of Table 3 prepared using vortex or microfluidic mixing.

FIG. 33A provides exemplary 3 and 5 lipid LNPs.

FIG. 33B provides lung transfection data using the LNPs of FIG. 33A.

FIG. 34A provides exemplary 2, 3 and 5 lipid LNPs.

FIGS. 34B and 34C provide lung transfection data using, and particle size and encapsulation efficiency of the LNPs of FIG. 34A.

FIG. 35A provides exemplary 5 lipid LNPs prepared using different flow rates.

FIGS. 35B and 35C provide encapsulation percentage and lung transfection data using the LNPs of FIG. 35A.

FIG. 36A provides exemplary 3 lipid LNPs prepared using different flow rates.

FIGS. 36B and 36C provide lung transfection and body weight change data using the LNPs of FIG. 36A.

FIGS. 37A and 37B provide exemplary LNPs prepared by substituting SS-OP with a different ionizable lipid (DLin-MC3-DMA), including encapsulation percentages.

FIGS. 37C and 37D provide lung transfection and body weight change data using the LNPs of FIG. 37A.

FIG. 38A provides exemplary LNPs prepared by swapping out SS-OP for DLin-MC3-DMA in 3 lipid LNPs.

FIGS. 38B and 38C provide encapsulation percentage and lung transfection data using the LNPs of FIG. 38A.

FIG. 39A provides exemplary LNPs prepared by swapping out SS-OP for DLin-MC3-DMA in 5 lipid LNPs.

FIGS. 39B and 39C provide encapsulation percentage and lung transfection data using the LNPs of FIG. 39A.

FIG. 40A provides a depiction of titrating time that mRNA is adsorbed to form LNP in a 2-lipid formulation.

FIG. 40B provides body weight change data using the LNPs of FIG. 40A.

FIGS. 41A and 41B provide lung transfection and encapsulation percentage data of 2-lipid LNPs made while titrating mRNA:lipid ratio.

FIGS. 42A and 42B provide lung transfection and encapsulation percentage data of 3-lipid LNPs made while titrating N/P ratio.

FIGS. 43A and 43B provide lung transfection and body weight change data using the listed 2-lipid LNPS titrated with DMG-PEG2000.

FIG. 44 provides lung mean radiance in a comparison of microfluidic vs manual mixing of lipids in ethanol with malic acid buffer.

FIGS. 45A-45D provide formulation, transfection and IHC data comparing an MC3 formulation with a 5 lipid LNP composition of the present specification.

FIG. 46A provides exemplary 2, 3 and 5 lipid LNPs.

FIGS. 46B, 46C and 46D provide lung, liver and spleen cell transfection data using the LNPs of FIG. 46A.

FIG. 46E provides IHC data related to use of exemplary 2, 3 and 5 lipid LNPs in lung, liver and spleen tissue.

FIGS. 47A, 47B and 47C depict the pharmacokinetics of specific 2-lipid LNPs in plasma, lung and liver.

FIG. 48 depicts the pharmacokinetics of specific 5-lipid LNPs from Table 1.

FIGS. 49A and 49B depict biodistribution of exemplary 5-lipid LNPs from Table 1 when administered to a mammal.

FIGS. 50A, 50B and 50C depict biodistribution of 3-lipid LNP as described in FIG. 46A when administered to a mammal.

FIGS. 50D, 50E and 50F depict organ level bioluminescence related to the use of 3-lipid LNPs in mammals as described in Example 33.

FIG. 50G depicts biodistribution of exemplary 3-lipid LNPs as described in FIG. 46A when administered to a mammal.

FIGS. 51A and 51B provide data related to telomere extension and telomerase activity in lung epithelial cells using the LNP of Table 3.

FIG. 52 provides data related to telomerase activity in lung fibroblasts using the LNP of Table 3.

FIGS. 53A and 53B provide lung transfection and encapsulation efficiency data using the LNPs of Example 38.

FIGS. 54A and 54B provide lung transfection and encapsulation efficiency data using the LNPs of Example 39.

FIG. 55 demonstrates lung mean radiance of the freeze-dried (lyophilized) LNPs of Example 36.

FIG. 56 models optimal cationic lipid to ionizable lipid ratio (“C/I Ratio”) and optimal lipid nitrogen:polynucleotide phosphate ratio (“N/P ratio”) for LNPs containing either the SS-OP family or DLin-MC3-DMA family of ionizable lipids. The grey boxes indicate the optimal ranges of C/I ratio and N/P ratio. The horizontal dashed lines indicate thresholds of relative lung radiance selected so that the convex peak(s) of relative lung radiance in each graph is/are substantially above the dashed lines. The horizonal dotted line is the threshold of relative yield such that the convex curve of relative yield is substantially above the dashed line. The limits of the optimal ranges (gray boxes) are determined by the intersections of the curves of relative lung radiance and relative yield with the horizontal dashed or dotted threshold lines, respectively.

### DETAILED DESCRIPTION

For clarity of disclosure, and not by way of limitation, the detailed description of the invention is divided into the subsections that follow.

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, the term “approximately” or “about,” as applied to one or more values of interest, refers to a value that is similar in magnitude and/or within a similar range to a stated reference value. In certain embodiments, the term “approximately” or “about” may refer to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure.

As used herein, the term “and/or” may mean “and,” it may mean “or,” it may mean “exclusive-or,” it may mean “one,” it may mean “some, but not all,” it may mean “neither,” and/or it may mean “both.”

The terms “individual,” “subject,” and “patient” are used interchangeably herein and refer to any subject for whom treatment or therapy is desired. The subject may be a mammalian subject. Mammalian subjects include, e. g., humans, non-human primates, rodents, (e.g., rats, mice), lagomorphs (e.g., rabbits), ungulates (e.g., cows, sheep, pigs, horses, goats, and the like), etc. In some embodiments, the subject is a human. In some embodiments, the subject is a non-human primate, for example a cynomolgus monkey. In some embodiments, the subject is a companion or service animal (e.g. cats or dogs).

The term “nucleic acid” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating nucleic acid sequences which, upon being translated (if RNA) or transcribed and translated (if DNA), produce the same sequence of amino acid residues as the original nucleic acid sequence; or, in which the third position of one or more selected (or all) codons is substituted with mixed-base and/or deoxyinosine residues.

The term “nucleotide sequence” refers to a polymer of DNA or RNA that can be single- or double-stranded, optionally containing synthetic, non-natural or altered nucleotide bases capable of incorporation into DNA or RNA polymers.

As used herein, “ribonucleic acid” means a polymer of ribonucleotides.

As used herein, “mRNA species” means mRNA molecules with identical ribonucleotide lengths and sequences.

Telomeres are repetitive DNA sequences bound by proteins at the ends of each chromosome arm, that protect the chromosome tips by forming a loop. Telomeres shorten with cell division, and if they become too short to form the loop, the DNA tip is exposed, activating DNA damage signaling, P53/P21, causing cellular senescence and apoptosis. The enzyme telomerase reverse transcriptase (TERT) extends telomeres and is activated transiently in normal stem and progenitor cells periodically throughout life3.

Alveolar type 2 (AT2) cells are stem cells able to renew and repair the alveolar epithelium. Accumulating evidence implicates AT2 cell telomeres in the etiology and pathogenesis of idiopathic pulmonary fibrosis (IPF). In the telomere model of IPF, alveolar cell damage from diverse sources drives AT2 cells to divide to repair and regenerate the alveolae11, and since telomeres shorten when cells divide, eventually AT2 cell telomeres become “critically short”, meaning that the protective telomere loop can no longer form, P53 and P21 are chronically activated, and deleterious consequences ensue. The model implies that individuals born with short telomeres, or predisposed to having short telomeres, would be predisposed to IPF, and indeed, telomere lengths in IPF patient leukocyte average below the 10th percentile of the general population, and 15% of familial IPF patients carry loss-of-function mutations in telomere maintenance genes, most commonly telomerase reverse transcriptase (TERT), the enzyme that extends telomeres and is expressed by alveolar epithelial AT2 cells. Independent of TERT mutation status, IPF patient lung tissues have decreased TERT mRNA and TERT protein levels. The model also suggests that AT2 cell telomeres would be relatively short within an individual compared to other cell types, and indeed, in IPF patients, telomeres are shorter in AT2 cells than other lung cells including myofibroblasts, and further, AT2 cell telomeres are shorter in fibrotic regions than non-fibrotic regions.

Short telomeres cause cellular senescence, and IPF patient lungs exhibit increased markers of senescence including P21. Short telomeres cause AT2 cell apoptosis and IPF patient lungs have depleted AT2 cell numbers. Consistent with deficient alveolar repair by AT2 cells due to telomere-induced senescence and apoptosis, IPF patients have elevated serum markers of epithelial damage such as CA-125 and SP-D.

Consistent with a role of short telomere-induced senescence in altering intercellular signaling, IPF patients also have elevated levels of inflammatory molecules (SASPs) such as TGFβ, implicated in lung fibroblast activation and conversion to myofibroblasts. Consistent with short telomere-induced senescence impairing successful AT2 differentiation, IPF patient lungs also accumulate a transitional cellular phenotype with a senescent profile.

Consistent with these consequences of short telomeres, longer telomere length in IPF patients is associated with transplant-free survival independent of age, sex, forced vital capacity, or diffusing capacity of carbon monoxide. Thus, in the telomere model of IPF, alveolar epithelial cell telomeres serve as an etiologic nexus, first integrating alveolar damage over time until they become critically short, and thereafter causing multiple pathogenic consequences culminating in death.

The telomere model of IPF motivates exploration of telomere extension. To avoid the risks of constitutive telomerase activity associated with cancer and AAV delivery of TERT DNA, here we focus on a method that only increases telomerase activity transiently, as stem cells do periodically throughout the lifespan.

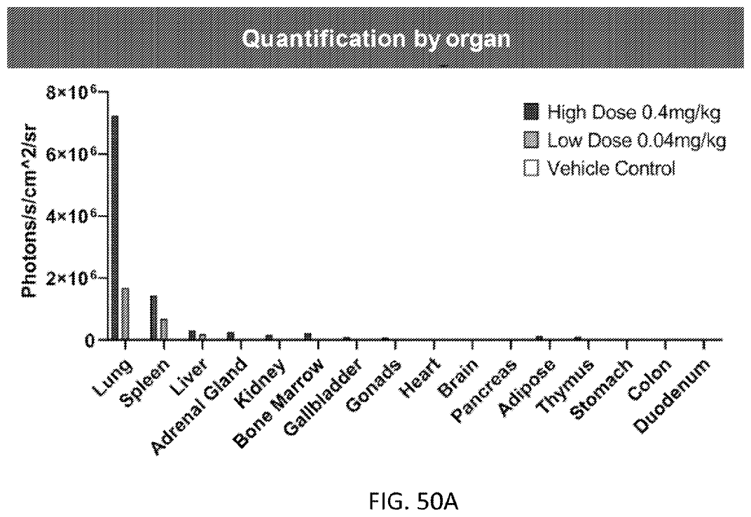

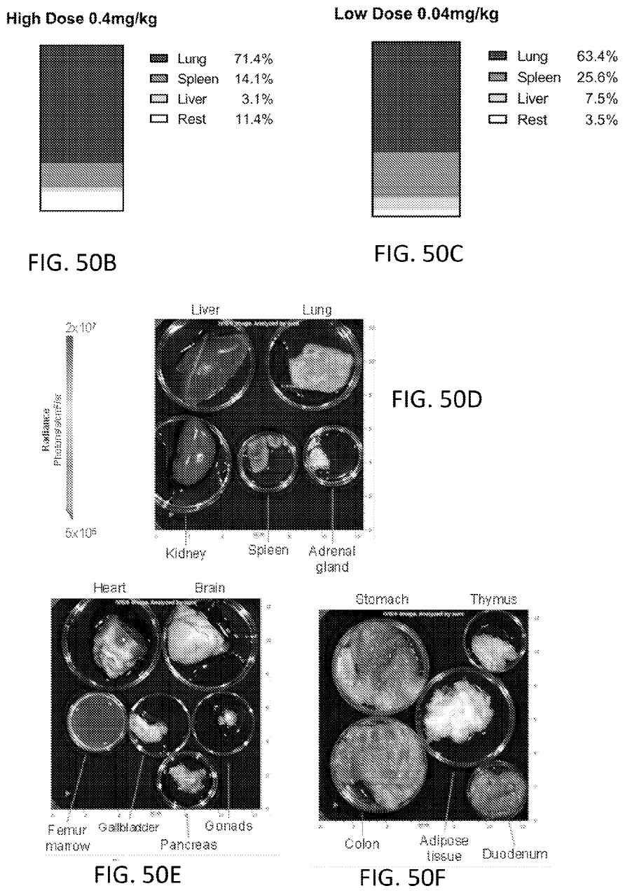

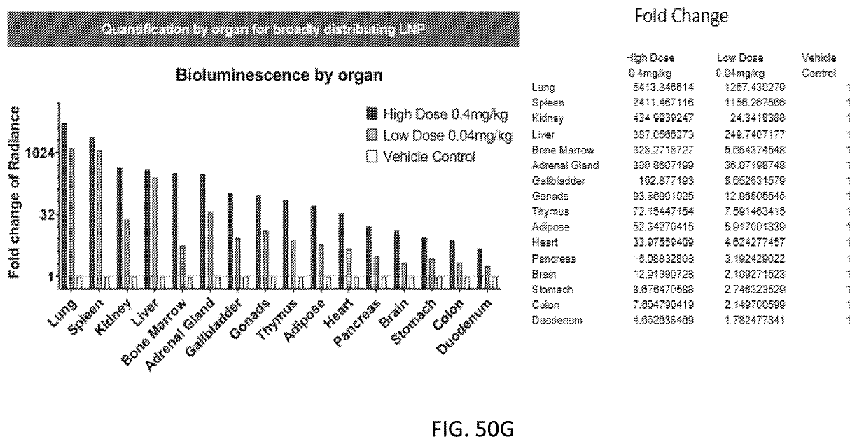

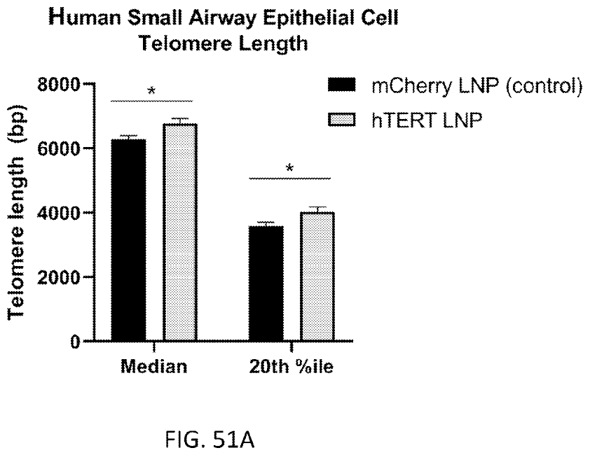

The data provided herein demonstrate or support, for example, pharmacokinetics of the exemplified LNPs in vivo, and particularly information and data related to the time course of concentration levels of a lipid from the LNPs in the lung, liver, and/or serum. In embodiments described herein, LNP compositions and formulations are provided that are rapidly cleared from the subject but have a longer half-life in the lung, lung tissues, and/or lung cells. In terms of rapid clearance, provided herein are LNP compositions that provide a ribonucleic acid payload that does not reside (90% to 100% has been cleared) in the tissues listed on FIG. 50A after about 9 to 11 hours after administration to the subject, except the lung. Conversely, in terms of rapid clearance, provided herein are LNP compositions that provide a ribonucleic acid payload that resides in the lung after about 9 to 11 hours after administration but is not present at all, or alternatively not present in any significant level, in one or more (including all) of spleen, live, adrenal gland, kidney, bone marrow, gallbladder, gonads, heart, brain, pancreas, adipose, thymus, stomach, colon and/or duodenum tissues.

The data provided herein also demonstrate or support, for example, the use of LNPs in which the mRNA encodes telomerase. Specifically provided herein are demonstrations of telomere extension in: alveolar cells in lungs in mice, and AT2 cells in alveoli of lungs of mice. Also provided is a demonstration of increased colony formation capacity of human primary alveolar epithelial cells by treatment with TERT mRNA LNPs.

Also provided is a demonstration of pharmacodynamics of TERT mRNA LNPs, specifically a time course of telomerase activity in lung tissue following intravenous infusion of TERT mRNA LNPs in mice.

Also provided are efficacy data of TERT mRNA LNPs in treating lung fibrosis in vivo, specifically improvement in lung function (FEV0.1 and FVC), reduction of lung fibrosis, increase in normo-aerative lung ratio, and improvement in lung architecture (alveolar density or number, circularity, and diameter).

Also provided herein are methods manufacture of LNPs. In one specific example, the LNPs are manufactured in a manner that forms the LNP prior to addition of nucleic acid. In specific embodiments thereof, such LNPs are 2, 3 or 5 lipids LNPs described herein and the method employs vortex mixing during the addition of the nucleic acid.

The present inventors have found that in LNPs adapted to work according to the presently contemplated methods and uses, the ratios of cationic lipid to ionizable lipid (e.g. SSOP:DOTAP or MC3:DOTAP) are unpredictable based on the specific constituents of the LNP. Specifically, while not intending to be bound by any specific theory, experimentally it appears that the particular ionizable lipid in the LNP affects the cationic lipid to ionizable lipid ratio in a manner that requires experimentation to confirm. The experimental evidence provided herein provides support for a specific cationic lipid to ionizable lipid ratio, or specific ratio range, based on the identified ionizable lipid in the LNP. The tables and Figures provided herein, for example Table 8, provide additional examples of specific ratios of cationic lipid to ionizable lipid of 3- and 5-lipid LNPs contemplated herein. In certain embodiments the cationic lipid to ionizable lipid ratio is at or about 0.8 and the ionizable lipid is SS-OP. In certain related embodiments the LNP has a cationic lipid to ionizable lipid ratio at or about 0.8 and is composed of 3 or 5 lipids. Also in certain related embodiments, the LNP has a cationic lipid to ionizable lipid ratio at or about 0.8 and is composed of 3 or 5 lipids, including DOTAP. Also in certain related embodiments, the LNP has a cationic lipid to ionizable lipid ratio at or about 0.8 and is composed of 3 or 5 lipids, including a pegylated lipid (e.g., DMG-PEG2000) and DOTAP.

Also in certain embodiments the cationic lipid to ionizable lipid ratio is at or about 1.8 and the ionizable lipid is SS-OP. In certain related embodiments the LNP has a cationic lipid to ionizable lipid ratio at or about 1.8 and is composed of 5 lipids. Also in certain related embodiments, the LNP has a cationic lipid to ionizable lipid ratio at or about 1.8 and is composed of 5 lipids, including DOTAP and DOPC. Also in certain related embodiments, the LNP has a cationic lipid to ionizable lipid ratio at or about 1.8 and is composed of 5 lipids, including a pegylated lipid (e.g., DMG-PEG2000), DOPC and DOTAP.

Also in certain embodiments the cationic lipid to ionizable lipid ratio is at or about 1.5 and the ionizable lipid is DLin-MC3-DMA. In certain related embodiments the LNP has a cationic lipid to ionizable lipid ratio is at or about 1.5 and is composed of 3 lipids. Also in certain related embodiments, the LNP has a cationic lipid to ionizable lipid ratio at or about 1.5 and is composed of 3 lipids, including DOTAP. Also in certain related embodiments, the LNP has a cationic lipid to ionizable lipid ratio at or about 1.5 and is composed of 3 lipids, including a pegylated lipid (e.g., DMG-PEG2000) and DOTAP.

LNP compositions of the present disclosure include and encompass a variety of lipid ratios and lipid:mRNA ratios (lipid:mRNA ratios are also referred to herein as “N/P ratio”). When mentioned herein LNP compositions refers to either or both the final LNP composition or the formulation used to produce the LNP which will be evident based on the context in which the phrase is used. Specific examples of contemplated compositions are provided in the Examples, including the supporting Figures. Particularly preferred LNP compositions of the present disclosure are provided, for example, in Table 8 and other tables, Figures and descriptions provided herein.

According to preferred embodiments herein, nucleic acid containing LNP compositions composed of 5 different lipids, including an ionizable lipid and a cationic lipid, and having a N/P ratio of between 10 and 30 are provided. Table 8 provides some such exemplary compositions. Often according to such embodiments such LNP compositions are further characterized as having a cationic lipid to ionizable lipid ratio of between 0.8 to 1.8. Also according to such embodiments such LNP compositions are further characterized as having a cationic lipid to ionizable lipid ratio of at or about 0.8. Also according to such embodiments such LNP compositions include SS-OP as the ionizable lipid in the composition. Also according to such embodiments such LNP compositions are used in vivo in a mammal subject and target lung tissue and/or lung cells in a manner that provides for transfection of the nucleic acid payload in the lung tissue and/or lung cells. Thus, such compositions are contemplated herein as adapted to target lung tissue and/or lung cells. Also according to such embodiments such LNP compositions preferentially target lung tissues and/or lung cells over other tissues in the subject, including for example spleen, live, adrenal gland, kidney, bone marrow, gallbladder, gonads, heart, brain, pancreas, adipose, thymus, stomach, colon and/or duodenum tissues. Also according to such embodiments such LNP compositions have an increased half-life in lung tissues and/or lung cells versus the half life in over other tissues in the subject, including for example spleen, live, adrenal gland, kidney, bone marrow, gallbladder, gonads, heart, brain, pancreas, adipose, thymus, stomach, colon and/or duodenum tissues. According to such embodiments such LNP compositions have a half-life in lung tissues or over 30 hours, or between 30 hours and 40 hours, or about 37 hours.

Also according to preferred embodiments herein, nucleic acid containing LNP compositions composed of 3 different lipids, including an ionizable lipid and a cationic lipid, and having a N/P ratio of between 4 and 24 are provided, or an N/P ratio of between 4.5 and 12. Table 8 provides some such exemplary compositions. Often according to such embodiments such LNP compositions are further characterized as having a cationic lipid to ionizable lipid ratio of between 0.8 to 1.5. Also according to such embodiments such LNP compositions are further characterized as having a cationic lipid to ionizable lipid ratio of at or about 0.8. Also according to such embodiments such LNP compositions include SS-OP as the ionizable lipid in the composition and a cationic lipid to ionizable lipid ratio of at or about 0.8. Also according to such embodiments such LNP compositions include DLin-MC3-DMA as the ionizable lipid in the composition and a cationic lipid to ionizable lipid ratio of at or about 1.5, and optionally an N/P ratio of at or about 4.5. Also according to such embodiments such LNP compositions are used in vivo in a mammal subject and target lung tissue and/or lung cells in a manner that provides for transfection of the nucleic acid payload in the lung tissue and/or lung cells. Thus, such compositions are contemplated herein as adapted to target lung tissue and/or lung cells. Also according to such embodiments such LNP compositions preferentially target lung tissues and/or lung cells over other tissues in the subject, including for example spleen, live, adrenal gland, kidney, bone marrow, gallbladder, gonads, heart, brain, pancreas, adipose, thymus, stomach, colon and/or duodenum tissues. Also according to such embodiments such LNP compositions have an increased half-life in lung tissues and/or lung cells versus the half life in over other tissues in the subject, including for example spleen, live, adrenal gland, kidney, bone marrow, gallbladder, gonads, heart, brain, pancreas, adipose, thymus, stomach, colon and/or duodenum tissues. According to such embodiments such LNP compositions have a half-life in lung tissues of over 5 hours, over 30 hours, or between 30 hours and 40 hours, or about 37 hours.

Also according to preferred embodiments herein, nucleic acid containing LNP compositions composed of 3 or 5 different lipids including DOTAP, including an ionizable lipid and a cationic lipid, and having a N/P ratio of between 4 and 24 are provided, or an N/P ratio of at or about 12. Also according to preferred embodiments herein, nucleic acid containing LNP compositions composed of 3 different lipids including DLin-MC3-DMA, including an ionizable lipid and a cationic lipid, and having a N/P ratio of between 4 and 24 are provided, or an N/P ratio of at or about 4.5.

Also according to frequently included embodiments contemplated herein, nucleic acid containing LNP compositions composed of 2 different lipids having a N/P ratio of at or about 4 are provided. Preferably, in such embodiments one of the two lipids is or comprises DOTAP. Also according to such embodiments such LNP compositions are used in vivo in a mammal subject and target lung tissue and/or lung cells in a manner that provides for transfection of the nucleic acid payload in the lung tissue and/or lung cells. Thus, such compositions are contemplated herein as adapted to target lung tissue and/or lung cells. Also according to such embodiments such LNP compositions preferentially target lung tissues and/or lung cells over other tissues in the subject, including for example spleen, live, adrenal gland, kidney, bone marrow, gallbladder, gonads, heart, brain, pancreas, adipose, thymus, stomach, colon and/or duodenum tissues. Also according to such embodiments such LNP compositions have an increased half-life in lung tissues and/or lung cells versus the half life in over other tissues in the subject, including for example spleen, live, adrenal gland, kidney, bone marrow, gallbladder, gonads, heart, brain, pancreas, adipose, thymus, stomach, colon and/or duodenum tissues. According to such embodiments such LNP compositions have a half-life in lung tissues or over 30 hours, or between 30 hours and 40 hours, or about 37 hours.

Also provided herein are methods of producing and/or storing LNP compositions. It has been found by the inventors that sucrose is a surprisingly good cryoprotectant for the presently contemplated LNP compositions. Specifically, it has been found that sucrose included at a range of between 5% to 25% of the final composition provides for preferred levels of cryoprotection. In specific embodiments the cryoprotectant (e.g., sucrose) is included at or about 15% of the final composition.

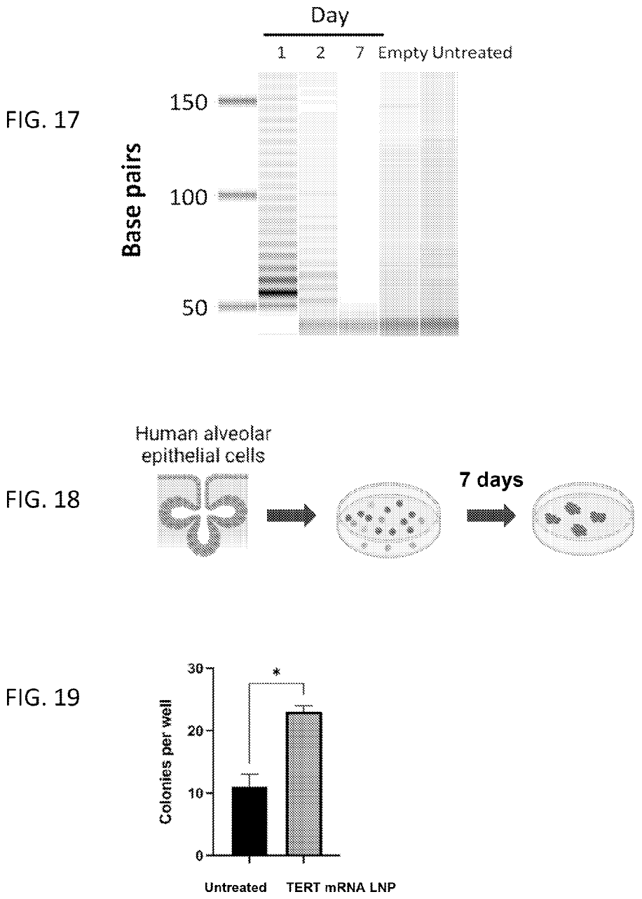

Delivery of synthetic nucleoside-modified mRNA encoding TERT (TERT mRNA) enables transient elevation of telomerase activity which extends telomeres in hours sufficiently to reverse years of telomere shortening. Importantly for safety, the mRNA and resulting TERT protein are degraded within hours and days, respectively, and the resulting telomerase activity lasts a few days (FIG. 17). Thus delivery of synthetic TERT mRNA recapitulates normal telomere extension by endogenous TERT mRNA as occurs in stem cells periodically throughout life. Immediately after the treatment ends, the extended telomeres resume shortening at their normal rate. Recent advances in lipid nanoparticle (LNP) vehicles enable delivery of mRNA to up to over 90% of lung epithelial cells via intravenous (IV) infusion with high tolerability.

Synthetic mRNAs

A synthetic ribonucleic acid (RNA) as used herein may refer to any RNA sequence comprising a mutation (point or deletion) or additional nucleotides not found in the wild type sequence. For example, a messenger RNA (mRNA) may refer to a wild type sequence encoding a human sequence, flanked by the addition of 1, 2, 3, 10, 100 or more nucleotides. Similarly, the nucleotides themselves may encode amino acids distinct from the wild type, or be modified to reduce immunogenicity in the cell or tissue. An mRNA sequence in some embodiments may comprise any of the following modifications, including but not limited to an untranslated region (UTR), a 5′ cap, and a poly-adenosine tail. In some embodiments, the RNA may be circular and/or self-replicating.

Illustrative methods of making circular mRNAs are provided in Chen et al. Science. 1995 Apr. 21; 268(5209):415-7; Perriman R. (2002) Circular mRNA Encoding for Monomeric and Polymeric Green Fluorescent Protein. In: Hicks B. W. (eds) Green Fluorescent Protein. Methods in Molecular Biology, vol 183. Humana Press; Wang et al. RNA. 2015 February; 21(2):172-9. doi: 10.1261/rna.048272.114. Epub 2014 Dec. 1; Wesselhoeft et al. Nat Commun. 2018 Jul. 6; 9(1):2629; and Wesselhoeft et al. Mol Cell. 2019 May 2; 74(3):508-520.e4. Illustrative methods of making self-replicating mRNAs are provided in Tews B. A., Meyers G. (2017) Self-Replicating RNA. In: Kramps T., Elbers K. (eds) RNA Vaccines. Methods in Molecular Biology, vol 1499. Humana Press; Leyman et al. Mol Pharm. 2018 Feb. 5; 15 (2):377-384; and Huysmans et al. Mol Ther Nucleic Acids. 2019 Sep. 6; 17:388-395.

In some embodiments, a mRNA may comprise a codon-optimized sequence. In some embodiments, a mRNA may comprise a uridine depleted sequence.

In some embodiments, the 5′ cap of the ribonucleic acid is a non-immunogenic cap. In some embodiments, the 5′ cap may increase the translation of the ribonucleic acid. In some embodiments, the 5′ cap may be treated with phosphatase to modulate the innate immunogenicity of the ribonucleic acid. In some embodiments, the 5′ cap is an anti-reverse cap analog (“ARCA”), such as a 3′-O-Me-m7G(5′)ppp(5′)G RNA cap structure analog. In some embodiments, the 5′ cap is m7G(5′)ppp(5′)(2′OmeA)pG (also known as CleanCap© AG). In some embodiments, the 5′ cap is m7(3′OmeG)(5′)ppp(5′)(2′OmeA)pG (also known as CleanCap®AG (3′ OMe)).

The above features, or others, may increase translation of the protein encoded by the ribonucleic acid, may increase or decrease the stability of the ribonucleic acid itself in a cell type-specific or cell type-independent manner, or may do both. In some embodiments, the 5′ UTR and/or the 3′ UTR are from a gene that has a very stable mRNA and/or an mRNA that is rapidly translated, for example, α-globin or β-globin, c-fos, or tobacco etch virus. In some embodiments, the 5′ UTR and 3′ UTR are from different genes or are from different species than the species into which the compositions are being delivered. The UTRs may also be assemblies of parts of UTRs from the mRNAs of different genes, where the parts are selected to achieve a certain combination of stability and efficiency of translation. The UTRs may also comprise designed sequences that confer properties to the RNA such as cell type-specific stability or cell type-independent stability.

The ribonucleic acids of the present disclosure may comprise one or more modified nucleosides, and/or comprise primary sequences of nucleosides, that modulate translation, stability, or immunogenicity of the RNA. Most mature RNA molecules in eukaryotic cells contain nucleosides that are modified versions of the canonical unmodified RNA nucleosides, adenine, cytidine, guanosine, and uridine. For example, the 5′ cap of mature RNA comprises a modified nucleoside, and other modified nucleosides often occur elsewhere in the RNA. Those modifications may prevent the RNA from being recognized as a foreign RNA. Synthetic RNA molecules made using certain nucleosides are much less immunogenic than unmodified RNA. The immunogenicity can be reduced even further by purifying the synthetic mRNA, for example by using high performance liquid chromatography (HPLC). The modified nucleosides may be, for example, chosen from the nucleosides listed below. The nucleosides are, in some embodiments, pseudouridine, 1-methylpseudouridine, 2-thiouridine, 5-methoxyuridine, or 5-methylcytidine. The primary sequence may be modified in ways that increase or decrease immunogenicity. Under some circumstances, it may be desirable for the modified RNA to retain some immunogenicity.

Accordingly, in some embodiments, the ribonucleic acids of the instant compositions comprise a 1-methylpseudouridine, pseudouridine, a 5-methoxyuridine (5-moU), a 2-thiouridine, a 5-methylcytidine, or another modified nucleoside. Modified nucleosides found in eukaryotic cells include m1A 1-methyladenosine, m6A N6-methyladenosine, Am 2′-O-methyladenosine, i6A N6-isopentenyladenosine, io6A N6-(cis-hydroxyisopentenyl) adenosine, ms2io6A 2-methylthio-N6-(cis-hydroxyisopentenyl) adenosine, g6A N6-glycinylcarbamoyladenosine, t6A N6-threonylcarbamoyladenosine, ms2t6A 2-methylthio-N6-threonyl carbamoyladenosine, Ar(p) 2′-O-ribosyladenosine (phosphate), m6 2A N6,N6-dimethyladenosine, m6Am N6,2′-O-dimethyladenosine, m6 2Am N6,N6,2′-O-trimethyladenosine, m1Am 1,2′-O-dimethyladenosine, m3C 3-methylcytidine, m5C 5-methylcytidine, Cm 2′-O-methylcytidine, ac4C N4-acetylcytidine, f5C 5-formylcytidine, m4C N4-methylcytidine, hm5C 5-hydroxymethylcytidine, f5Cm 5-formyl-2′-O-methylcytidine, m1G 1-methylguanosine, m2G N2-methylguanosine, m7G 7-methylguanosine, Gm 2′-O-methylguanosine, m2 2G N2,N2-dimethylguanosine, Gr(p) 2′-O-ribosylguanosine (phosphate), yW wybutosine, o2yW peroxywybutosine, OhyW hydroxywybutosine, OhyW* undermodified hydroxywybutosine, imG wyosine, m2,7G N2,7-dimethylguanosine, m2,2,7G N2,N2,7-trimethylguanosine I inosine, m1I 1-methylinosine, Im 2′-O-methylinosine, Q queuosine, galQ galactosyl-queuosine, manQ mannosyl-queuosine, Ψ pseudouridine, D dihydrouridine, m5U 5-methyluridine, Um 2′-O-methyluridine, m5Um 5,2′-O-dimethyluridine, m1Ψ′ 1-methylpseudouridine, Ψm 2′-O-methylpseudouridine, s2U 2-thiouridine, ho5U 5-hydroxyuridine, chm5U 5-(carboxyhydroxymethyl)uridine, mchm5U 5-(carboxyhydroxymethyl)uridine, methyl ester mcm5U 5-methoxycarbonylmethyluridine, mcm5Um 5-methoxycarbonylmethyl-2′-O-methyluridine, mcm5s2U 5-methoxycarbonylmethyl-2-thiouridine, ncm5U 5-carbamoylmethyluridine, ncm5Um 5-carbamoylmethyl-2′-O-methyluridine, cmnm5U 5-carboxymethylaminomethyluridine, m3U 3-methyluridine, m1acp3′Ψ 1-methyl-3-(3-amino-3-carboxypropyl) pseudouridine, cm5U 5-carboxymethyluridine, m3Um 3,2′-O-dimethyluridine, m5D 5-methyldihydrouridine, τm5U 5-taurinomethyluridine, τm5s2U 5-taurinomethyl-2-thiouridine, 2-Aminoadenosine, 2-Amino-6-chloropurineriboside, 8-Azaadenosine, 6-Chloropurineriboside, 5-Iodocytidine, 5-Iodouridine, Inosine, 2′-O-Methylinosine, Xanthosine, 4-Thiouridine, O6-Methylguanosine, 5,6-Dihydrouridine, 2-Thiocytidine, 6-Azacytidine, 6-Azauridine, 2′-O-Methyl-2-aminoadenosine, 2′-O-Methylpseudouridine, N1-Methyladenosine, 2′-O-Methyl-5-methyluridine, 7-Deazaguanosine, 8-Azidoadenosine, 5-Bromocytidine, 5-Bromouridine, 7-Deazaadenosine, 5-Aminoallyluridine, 5-Aminoallylcytidine, 8-Oxoguanosine, 2-Aminopurine-riboside, Pseudoisocytidine, N1-Methylpseudouridine, 5,6-Dihydro-5-Methyluridine, N6-Methyl-2-Aminoadenosine, 5-Carboxycytidine, 5-Hydroxymethyluridine, Thienoguanosine, 5-Hydroxycytidine, 5-Formyluridine, 5-Carboxyuridine, 5-Methoxyuridine, 5-Methoxycytidine, Thienouridine, 5-Carboxymethylesteruridine, Thienocytidine, 8-Oxoadenoosine, Isoguanosine, N1-Ethylpseudouridine, N1-Methyl-2′-O-Methylpseudouridine, N1-Methoxymethylpseudouridine, N1-Propylpseudouridine, 2′-O-Methyl-N6-Methyladenosine, 2-Amino-6-C1-purine-2′-deoxyriboside, 2-Amino-2′-deoxyadenosine, 2-Aminopurine-2′-deoxyriboside, 5-Bromo-2′-deoxycytidine, 5-Bromo-2′-deoxyuridine, 6-Chloropurine-2′-deoxyriboside, 7-Deaza-2′-deoxyadenosine, 7-Deaza-2′-deoxyguanosine, 2′-Deoxyinosine, 5-Propynyl-2′-deoxycytidine, 5-Propynyl-2′-deoxyuridine, 5-Fluoro-2′-deoxyuridine, 5-Iodo-2′-deoxycytidine, 5-Iodo-2′-deoxyuridine, N6-Methyl-2′-deoxyadenosine, 5-Methyl-2′-deoxycytidine, 06-Methyl-2′-deoxyguanosine, N2-Methyl-2′-deoxyguanosine, 8-Oxo-2′-deoxyadenosine, 8-Oxo-2′-deoxyguanosine, 2-Thiothymidine, 2′-Deoxy-P-nucleoside, 5-Hydroxy-2′-deoxycytidine, 4-Thiothymidine, 2-Thio-2′-deoxycytidine, 6-Aza-2′-deoxyuridine, 6-Thio-2′-deoxyguanosine, 8-Chloro-2′-deoxyadenosine, 5-Aminoallyl-2′-deoxycytidine, 5-Aminoallyl-2′-deoxyuridine, N4-Methyl-2′-deoxycytidine, 2′-Deoxyzebularine, 5-Hydroxymethyl-2′-deoxyuridine, 5-Hydroxymethyl-2′-deoxycytidine, 5-Propargylamino-2′-deoxycytidine, 5-Propargylamino-2′-deoxyuridine, 5-Carboxy-2′-deoxycytidine, 5-Formyl-2′-deoxycytidine, 5-[(3-Indolyl) propionamide-N-allyl]-2′-deoxyuridine, 5-Carboxy-2′-deoxyuridine, 5-Formyl-2′-deoxyuridine, 7-Deaza-7-Propargylamino-2′-deoxyadenosine, 7-Deaza-7-Propargylamino-2′-deoxyguanosine, Biotin-16-Aminoallyl-2′-dUTP, Biotin-16-Aminoallyl-2′-dCTP, Biotin-16-Aminoallylcytidine, N4-Biotin-OBEA-2′-deoxycytidine, Biotin-16-Aminoallyluridine, Dabcyl-5-3-Aminoallyl-2′-dUTP, Desthiobiotin-6-Aminoallyl-2′-deoxycytidine, Desthiobiotin-16-Aminoallyl-Uridine, Biotin-16-7-Deaza-7-Propargylamino-2′-deoxyguanosine, Cyanine 3-5-Propargylamino-2′-deoxycytidine, Cyanine 3-6-Propargylamino-2′-deoxyuridine, Cyanine 5-6-Propargylamino-2′-deoxycytidine, Cyanine 5-6-Propargylamino-2′-deoxyuridine, Cyanine 3-Aminoallylcytidine, Cyanine 3-Aminoallyluridine, Cyanine 5-Aminoallylcytidine, Cyanine 5-Aminoallyluridine, Cyanine 7-Aminoallyluridine, 2′-Fluoro-2′-deoxyadenosine, 2′-Fluoro-2′-deoxycytidine, 2′-Fluoro-2′-deoxyguanosine, 2′-Fluoro-2′-deoxyuridine, 2′-O-Methyladenosine, 2′-O-Methylcytidine, 2′-O-Methylguanosine, 2′-O-Methyluridine, Puromycin, 2′-Amino-2′-deoxycytidine, 2′-Amino-2′-deoxyuridine, 2′-Azido-2′-deoxycytidine, 2′-Azido-2′-deoxyuridine, Aracytidine, Arauridine, 2′-Azido-2′-deoxyadenosine, 2′-Amino-2′-deoxyadenosine, Araadenosine, 2′-Fluoro-thymidine, 3′-O-Methyladenosine, 3′-O-Methylcytidine, 3′-O-Methylguanosine, 3′-O-Methyluridine, 2′-Azido-2′-deoxyguanosine, Araguanosine, 2′-Deoxyuridine, 3′-O-(2-nitrobenzyl)-2′-Deoxyadenosine, 3′-O-(2-nitrobenzyl)-2′-Deoxyinosine, 3′-Deoxyadenosine, 3′-Deoxyguanosine, 3′-Deoxycytidine, 3′-Deoxy-5-Methyluridine, 3′-Deoxyuridine, 2′,3′-Dideoxyadenosine, 2′,3′-Dideoxyguanosine, 2′,3′-Dideoxyuridine, 2′,3′-Dideoxythymidine, 2′,3′-Dideoxycytidine, 3′-Azido-2′,3′-dideoxyadenosine, 3′-Azido-2′,3′-dideoxythymidine, 3′-Amino-2′,3′-dideoxyadenosine, 3′-Amino-2′,3′-dideoxycytidine, 3′-Amino-2′,3′-dideoxyguanosine, 3′-Amino-2′,3′-dideoxythymidine, 3′-Azido-2′,3′-dideoxycytidine, 3′-Azido-2′,3′-dideoxyuridine, 5-Bromo-2′,3′-dideoxyuridine, 2′,3′-Dideoxyinosine, 2′-Deoxyadenosine-5′-O-(1-Thiophosphate), 2′-Deoxycytidine-5′-O-(1-Thiophosphate), 2′-Deoxyguanosine-5′-O-(1-Thiophosphate), 2′-Deoxythymidine-5′-O-(1-Thiophosphate), Adenosine-5′-O-(1-Thiophosphate), Cytidine-5′-O-(1-Thiophosphate), Guanosine-5′-O-(1-Thiophosphate), Uridine-5′-O-(1-Thiophosphate), 2′,3′-Dideoxyadenosine-5′-O-(1-Thiophosphate), 2′,3′-Dideoxycytidine-5′-O-(1-Thiophosphate), 2′,3′-Dideoxy guanosine-5′-O-(1-Thiophosphate), 3′-Deoxythymidine-5′-O-(1-Thiophosphate), 3′-Azido-2′,3′-dideoxythymidine-5′-O-(1-Thiophosphate), 2′,3′-Dideoxyuridine-5′-O-(1-Thiophosphate), 2′-Deoxyadenosine-5′-O-(1-Boranophosphate), 2′-Deoxycytidine-5′-O-(1-Boranophosphate), 2′-Deoxy guanosine-5′-O-(1-Boranophosphate), and 2′-Deoxythymidine-5′-O-(1-Boranophosphate).

Without intending to be bound by theory, the presence of the modified nucleosides, and/or sequences of nucleosides that alter secondary structure of the RNA and/or binding of RNA to RNA binding proteins or microRNA, may enable mRNA to avoid activation of an immune response mediated by various receptors, including the Toll-like receptors and RIG-1. Non-immunogenic mRNA has been used as a therapeutic agent in mice via topical delivery. Kormann et al. (2011) Nature Biotechnology 29:154-157. In some embodiments, the ribonucleic acids comprise more than one of the above nucleosides or combination of the above nucleosides. In some embodiments, the ribonucleic acids comprise 1-methylpseudouridine, 5-methoxyuridine, or pseudouridine and 5-methylcytidine.

In some embodiments, an immune response to the mRNA may be desired, and the RNA may be modified to induce an optimal level of innate immunity. In other embodiments, an immune response to the mRNA may not be desired, and the RNA may be modified in order to minimize such a reaction. The RNA can be modified for either situation.

The ribonucleic acid molecules can be synthetic ribonucleic acids. The term “synthetic”, as used herein, can mean that the ribonucleic acids are in some embodiments prepared using the tools of molecular biology under the direction of a human, for example as described below. The synthetic ribonucleic acids may, for example, be prepared by in vitro synthesis using cellular extracts or purified enzymes and nucleic acid templates. The synthetic ribonucleic acids may in some embodiments be prepared by chemical synthesis, either partially or completely. Alternatively, or in addition, the synthetic ribonucleic acids may in some embodiments be prepared by engineered expression in a cell, followed by disruption of the cell and at least partial purification of the ribonucleic acid.

The ribonucleic acids of the present disclosure may be prepared using a variety of techniques, as would be understood by one of ordinary skill in the art. In some embodiments, the ribonucleic acids may be prepared by in vitro synthesis. In some embodiments, the ribonucleic acids may be prepared by chemical synthesis. In some embodiments, the ribonucleic acids may be prepared by a combination of in vitro synthesis and chemical synthesis. As described above, the term “synthetic” should be understood to include ribonucleic acids that are prepared either by chemical synthesis, by in vitro synthesis, by expression in vivo and at least partial purification, or by a combination of such, or other, chemical or molecular biological methods.

The ribonucleic acids may, in some embodiments, be purified. As noted above, purification may reduce immunogenicity of the ribonucleic acids and may be advantageous in some circumstances. In some embodiments, the ribonucleic acids are purified by one or more of HPLC, DNAse treatment, protease treatment, or by affinity capture and elution.

In some embodiments, an mRNA sequence may be synthesized as an unmodified or modified mRNA. An mRNA may be modified to enhance stability and/or evade immune detection and degradation. A modified mRNA may include, for example, one or more of a nucleotide modification, a nucleoside modification, a backbone modification, a sugar modification, and/or a base modification. In some embodiments, the modified nucleoside is pseudouridine or a pseudouridine analog. In some embodiments, the pseudouridine analog is N-1-methylpseudouridine. In some embodiments, the modified nucleoside is 5-methoxyuridine. In some embodiments a modified nucleoside as used herein may comprise any of the moieties listed in Table 2.

TABLE 2
Common name

pseudouridine
N-1-methylpseudouridine
5-methoxyuridine
1,2′-O-dimethyladenosine
1-methyl-3-(3-amino-3-carboxypropyl) pseudouridine
1-methyladenosine
1-methylguanosine
1-methylinosine
1-methylpseudouridine
2,2-dimethyl-guanosine
2′,3′-dideoxyadenosine
2′,3′-Dideoxycytidine
2′,3′-Dideoxyguanosine
2′,3′-Dideoxyinosine
2′,3′-dideoxynucleosides
2′,3′-Dideoxythymidine
2′,3′-dideoxythymine
2′,3′-Dideoxyuridine
2,6-diaminopurine
2′-O-ribosyladenosine (phosphate)
2′-Amino-2′-deoxyadenosine
2-Amino-2′-deoxyadenosine
2′-Amino-2′-deoxyuridine
2-Amino-6-chloropurineriboside
2-Amino-6-Cl-purine-2′-deoxyriboside
2-aminoadenosine
2-Aminoadenosine
2-Aminopurine-2′-deoxyriboside
2-Aminopurine-riboside
2′-Azido-2′-deoxyadenosine
2′-Azido-2′-deoxycytidine
2′-Azido-2′-deoxyguanosine
2′-Azido-2′-deoxyuridine
2′-Deoxyinosine
2′-Deoxy-P-nucleoside
2′-Deoxyuridine
2′-Deoxyzebularine
2′-Fluoro-2′-deoxyadenosine
2′-Fluoro-2′-deoxycytidine
2′-Fluoro-2′-deoxyguanosine
2′-Fluoro-2′-deoxyuridine
2′-Fluoro-thymidine
2-methyl-adenosine
2-methyl-guanosine
2-methylthio-N⁶-(cis-hydroxyisopentenyl) adenosine
2-methylthio-N-6-isopentenyl-adenosine
2-methylthio-N⁶-threonylcarbamoyladenosine
2′-O-Methyl-2-aminoadenosine
2′-O-Methyl-5-methyluridine
2′-O-methyladenosine
2′-O-methylcytidine
2′-O-methylguanosine
2′-O-methylinosine
2′-O-Methyl-N6-Methyladenosine
2′-O-methylpseudouridine
2′-O-methyluridine
2′-O-ribosylguanosine (phosphate)
2-Thio-2′-deoxycytidine
2-Thiocytidine
2-Thiothymidine
2-thiouridine
3,2′-O-dimethyluridine
3′-Amino-2′,3′-dideoxyadenosine
3′-Amino-2′,3′-dideoxycytidine
3′-Amino-2′,3′-dideoxyguanosine
3′-Amino-2′,3′-dideoxythymidine
3′-Azido-2′,3′-dideoxyadenosine
3′-Azido-2′,3′-dideoxycytidine
3′-Azido-2′,3′-dideoxythymidine
3′-Azido-2′,3′-dideoxyuridine
3′-Deoxy-5-Methyluridine
3′-Deoxyadenosine
3′-deoxyadenosine (cordycepin)
3′-Deoxycytidine
3′-Deoxyguanosine
3′-deoxythymine
3′-Deoxyuridine
3-methylcytidine
3-methyluridine
3′-O-(2-nitrobenzyl)-2′-Deoxyadenosine
3′-O-(2-nitrobenzyl)-2′-Deoxyinosine
3′-O-Methyladenosine
3′-O-Methylcytidine
3′-O-Methylguanosine
3′-O-Methyluridine
4-acetyl-cytidine
4-Thiothymidine
4-Thiouridine
5-(carboxyhydroxymethyl) uridine methyl ester
5-(carboxyhydroxymethyl)uridine
5,2′-O-dimethyluridine
5,6-Dihydro-5-Methyluridine
5,6-Dihydrouridine
5-[(3-Indolyl)propionamide-N-allyl]-2′-deoxyuridine
5-Aminoallyl-2′-deoxycytidine
5-Aminoallyl-2′-deoxyuridine
5-Aminoallylcytidine
5-Aminoallyluridine
5-Bromo-2′,3′-dideoxyuridine
5-Bromo-2′-deoxycytidine
5-Bromo-2′-deoxyuridine
5-Bromocytidine
5-Bromouridine
5-carbamoylmethy1-2′-O-methyluridine
5-carbamoylmethyluridine
5-Carboxy-2′-deoxycytidine
5-Carboxycytidine
5-carboxymethylaminomethyl-2-thio-uridine
5-carboxymethylaminomethyluridine
5-Carboxymethylesteruridine
5-carboxymethyluridine
5-Carboxyuridine
5-Fluoro-2′-deoxyuridine
5-fluoro-uridine
5-Formyl-2′-deoxycytidine
5-Formy1-2′-deoxyuridine
5-formy1-2′-O-methylcytidine
5-formylcytidine
5-Formyluridine
5-Hydroxy-2′-deoxycytidine
5-Hydroxycytidine
5-Hydroxymethyl-2′-deoxycytidine
5-Hydroxymethyl-2′-deoxyuridine
5-hydroxymethylcytidine
5-Hydroxymethyluridine
5-hydroxyuridine
5-Iodo-2′-deoxycytidine
5-Iodo-2′-deoxyuridine
5-Iodocytidine
5-Iodouridine
5-methoxyaminomethyl-2-thio-uridine
5-methoxycarbonylmethy1-2′-O-methyluridine
5-methoxycarbonylmethyl-2-thiouridine
5′-methoxycarbonylmethyl-uridine
5-methoxycarbonylmethyluridine
5-Methoxycytidine
5-Methoxyuridine
5-methoxy-uridine
5-Methyl-2′-deoxycytidine
5-methyl-2-thio-uridine
5-methylaminomethyl-uridine
5-methylcytidine
5-methyldihydrouridine
5-methyluridine
5-Propargylamino-2′-deoxycytidine
5-Propargylamino-2′-deoxyuridine
5-Propynyl-2′-deoxycytidine
5-taurinomethy1-2-thiouridine
5-taurinomethyluridine
6-Aza-2′-deoxyuridine
6-Azacytidine
6-Azauridine
6-chloropurine riboside
6-Chloropurine-2′-deoxyriboside
6-O-methylguanosine
6-Thio-2′-deoxyguanosine
7-Deaza-2′-deoxyadenosine
7-Deaza-2′-deoxyguanosine
7-Deaza-7-Propargylamino-2′-deoxyadenosine
7-Deaza-7-Propargylamino-2′-deoxyguanosine
7-Deazaadenosine
7-Deazaguanosine
7-methylguanosine
7-methyl-guanosine
8-Azaadenosine
8-Azidoadenosine
8-Chloro-2′-deoxyadenosine
8-Oxo-2′-deoxyadenosine
8-Oxo-2′-deoxyguanosine
8-Oxoadenoosine
8-Oxoguanosine
a 2′-deoxynucleoside
ac4C N4-acetylcytidine
Am 2′-O-methyladenosine
an-O-methylnucleoside
Ar(p) 2′-O-ribosyladenosine (phosphate)
Araadenosine
Aracytidine
Araguanosine
Arauridin
benzimidazole riboside
beta-D-mannosyl-queosine
Biotin-16-7-Deaza-7-Propargylamino-2′-deoxyguanosine
Biotin-16-Aminoallyl-2′-dCTP
Biotin-16-Aminoallyl-2′-dUTP
Biotin-16-Aminoallylcytidine
Biotin-16-Aminoallyluridine
chm5U 5-(carboxyhydroxymethyl)uridine
2′-O-methylcytidine
5-carboxymethyluridine
5-carboxymethylaminomethyluridine
Cyanine 3-5-Propargylamino-2′-deoxycytidine
Cyanine 3-6-Propargylamino-2′-deoxyuridine
Cyanine 3-Aminoallylcytidine
Cyanine 3-Aminoallyluridine
Cyanine 5-6-Propargylamino-2′-deoxycytidine
Cyanine 5-6-Propargylamino-2′-deoxyuridine
Cyanine 5-Aminoallylcytidine
Cyanine 5-Aminoallyluridine
Cyanine 7-Aminoallyluridine
dihydrouridine
Dabcyl-5-3-Aminoallyl-2′-dUTP
Desthiobiotin-16-Aminoallyl-Uridine
Desthiobiotin-6-Aminoallyl-2′-deoxycytidine
dihydrouridine
5-formylcytidine
5-formyl-2′-O-methylcytidine
N6-glycinylcarbamoyladenosine
galactosyl-queuosine
2′-O-methylguanosine
2′-O-ribosylguanosine (phosphate)
5-hydroxymethylcytidine
5-hydroxyuridine
hydroxywybutosine
N6-isopentenyladenosine
2′-O-methylinosine
wyosine
inosine
N6-(cis-hydroxyisopentenyl)adenosine
Isoguanosine
l-methylguanosine
1-methyladenosine
1-methyl-3-(3-amino-3-carboxypropyl) pseudouridine
1,2′-O-dimethyladenosine
1-methylguanosine
1-methylinosine
1-methylpseudouridine
N2,N2-dimethylguanosine
N2,N2,7-trimethylguanosine I inosine
N2,7-dimethylguanosine
N2-methylguanosine
3-methylcytidine
3-methyluridine
3,2′-O-dimethyluridine
N4-methylcytidine
5-methylcytidine
5-methyldihydrouridine
5-methyluridine
5,2′-O-dimethyluridine
N6,N6-dimethyladenosine
N6,N6,2′-O-trimethyladenosine
N6-methyladenosine
N6,2′-O-dimethyladenosine
7-methylguanosine
mannosyl-queuosine
5-(carboxyhydroxymethyl)uridine
5-methoxycarbonylmethyl-2-thiouridine
5-methoxycarbonylmethyl-2′-O-methyluridine
5-methoxycarbonylmethyluridine
2-methylthio-N6-(cis-hydroxyisopentenyl) adenosine
2-methylthio-N6-threonyl carbamoyladenosine
N1-Ethylpseudouridine
N1-Methoxymethylpseudouridine
N1-Methyl-2′-O-Methylpseudouridine
N1-Methyladenosine
N1-Propylpseudouridine
N²,7-dimethylguanosine
N²,N²,7-trimethylguanosine
N²,N²-dimethylguanosine
N2-Methyl-2′-deoxyguanosine
N²-methylguanosine
N⁴-acetylcytidine
N4-Biotin-OBEA-2′-deoxycytidine
N4-Methyl-2′-deoxycytidine
N⁴-methylcytidine
N⁶-(cis-hydroxyisopentenyl)-adenosine
N⁶,2′-O-dimethyladenosine
N⁶,N⁶,2′-O-trimethyladenosine
N⁶,N⁶-dimethyladenosine
N⁶-glycinylcarbamoyladenosine
N⁶-isopentenyladenosine
N6-isopentenyl-adenosine
N6-Methyl-2-Aminoadenosine
N6-Methyl-2′-deoxyadenosine
N⁶-methyladenosine
N6-methyl-adenosine
N⁶-threonylcarbamoyladenosine
ncm5U 5-carbamoylmethyluridine
ncm5Um 5-carbamoylmethyl-2′-O-methyluridine
Nl-methyladenosine
N-uridine-5-oxyaceticacidmethylester
peroxywybutosine
O6-Methyl-2′-deoxyguanosine
O6-Methylguanosine
hydroxywybutosine
undermodified hydroxywybutosine
O-Methylpseudouridine
peroxywybutosine
Pseudoisocytidine
Puromycin
queosine
2-thiouridine
N6-threonylcarbamoyladenosine
Thienocytidine
Thienoguanosine
Thienouridine
2′-O-methyluridine
undermodified hydroxywybutosine
uridine-5-oxyaceticacid(v)
uridine-5-oxyaceticacidmethylester
wybutosine
wybutoxosine
wyosine
Xanthosine
5-taurinomethyl-2-thiouridine
5-taurinomethyluridine
2′-O-methylpseudouridine

In some embodiments, an RNA, e.g. an mRNA, may be synthesized from naturally occurring bases and/or base analogs (modified bases) including, but not limited to, purines (adenine (A), guanine (G)) or pyrimidines (thymine (T), cytosine (C), uracil (U)), and analogues and derivatives thereof, e.g. 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), pseudouridine, N-1-methyl-pseudouridine, dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, beta-D-mannosyl-queosine, wybutoxosine, and phosphoramidates, phosphorothioates, peptide nucleotides, methylphosphonates, 7-deazaguanosine, 5-methylcytosine and inosine.

In some embodiments, an RNA, e.g., an mRNA, may be synthesized from naturally occurring nucleosides and/or nucleoside analogs (modified nucleosides) including, but not limited to, nucleosides comprising adenosine (A), guanosine (G)) or pyrimidines (thymine (T), cytidine (C), uridine (U)), and nucleoside comprising analogues and derivatives thereof, e.g., 3′-deoxyadenosine (cordycepin), 3′-deoxyuridine, 3′-deoxycytosine, 3′-deoxyguanosine, 3′-deoxythymine, 2′,3′-dideoxynucleosides, 2′,3′-dideoxyadenosine, 2′,3′-dideoxyuridine, 2′,3′-dideoxycytosine, 2′,3′-dideoxyguanosine, 2′,3′-dideoxythymine, a 2′-deoxynucleoside, —O-methylnucleoside, 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouridine, N-1-methyl-pseudouridine, dihydro-uracil, 2-thio-uracil, 4-thio-uridine, 5-carboxymethylaminomethyl-2-thio-uridine, 5-(carboxyhydroxymethyl)-uridine, 5-fluoro-uridine, 5-bromo-uridine, 5-carboxymethylaminomethyl-uridine, 5-methyl-2-thio-uridine, 5-methyl-uridine, N-uridine-5-oxyacetic acid methyl ester, 5-methylaminomethyl-uridine, 5-methoxyaminomethyl-2-thio-uridine, 5′-methoxycarbonylmethyl-uracil, 5-methoxy-uracil, uracil-5-oxyacetic acid methyl ester, uracil-5-oxyacetic acid (v), 1-methyl-pseudouridine, queosine, beta-D-mannosyl-queosine, wybutoxosine, 7-deazaguanosine, 5-methylcytosine, and inosine.

The preparation of such base, nucleoside, nucleotide, and backbone analogues, modifications, and derivatives is known to a person skilled in the art e.g. from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530 and 5,700,642, all of which are incorporated by reference in their entirety.

In some embodiments, uracil nucleosides of the mRNA are about 80%, about 90%, 95%, 99%, or 100% depleted and replaced with a uracil nucleoside analog, e.g., pseudouridine, 5-methoxyuridine, or N-1-methyl-pseudouridine.

In some embodiments, an RNA may contain an RNA backbone modification. Typically, a backbone modification is a modification in which the phosphates of the backbone of the nucleotides contained in the RNA are chemically modified. Exemplary backbone modifications may include, but are not limited to, modifications in which the phosphodiester linkage is replaced with a member from the group consisting of peptides, methylphosphonates, methylphosphoramidates, phosphoramidates, phosphorothioates (e.g., cytidine 5′-O-(1-thiophosphate)), boranophosphates, and/or positively charged guanidimum groups, or other means of replacing the phosphodiester linkage.

In some embodiments, an RNA may contain sugar modifications. A sugar modification may include but is not limited to, 2′ O-methyl sugar modifications, 2′ fluoro sugar modifications (e.g. 2′-fluororibose), 3′ amino sugar modifications, 2′ thio sugar modifications, 2′-O-alkyl sugar modifications, 5-methylthioribose, and sugar modifications of 2′-deoxy-2′-fluoro-ribonucleotide (2′-fluoro-2′-deoxycytidine, 2′-fluoro-2′-deoxyuridine), 2′-deoxy-2′-deamine-ribonucleotide (2′-amino-2′-deoxycytidine, 2-amino-2′-deoxyuridine), 2′-O-alkylribonucleotide, 2′-deoxy-2′-C-alkylribonucleotide (2′-O-methylcytidine, 2′-methyluridine), 2′-C-alkylribonucleotide, and isomers thereof (2′-aracytidine, 2′-arauridine), or azidophosphates (2′-azido-2′-deoxycytidine, 2′-azido-2′-deoxyuridine).

In some embodiments, an RNA may be synthesized from one or more of the nucleotide triphosphates comprising any of the nucleosides and nucleotides disclosed herein, or any of the following nucleoside triphosphates: 2′-Deoxyadenosine-5′-O-(1-Thiotriphosphate), 2′-Deoxycytidine-5′-O-(1-Thiotriphosphate), 2′-Deoxyguanosine-5′-O-(1-Thiotriphosphate), 2′-Deoxythymidine-5′-O-(1-Thiotriphosphate), Adenosine-5′-O-(1-Thiotriphosphate), Cytidine-5′-O-(1-Thiotriphosphate), Guanosine-5′-O-(1-Thiotriphosphate), Uridine-5′-O-(1-Thiotriphosphate), 2′,3′-Dideoxyadenosine-5′-O-(1-Thiotriphosphate), 2′,3′-Dideoxycytidine-5′-O-(1-Thiotriphosphate), 2′,3′-Dideoxyguanosine-5′-O-(1-Thiotriphosphate), 3′-Deoxythymidine-5′-O-(1-Thiotriphosphate), 3′-Azido-2′,3′-dideoxythymidine-5′-O-(1-Thiotriphosphate), 2′,3′-Dideoxyuridine-5′-O-(1-Thiotriphosphate), 2′-Deoxyadenosine-5′-O-(1-Boranotriphosphate), 2′-Deoxycytidine-5′-O-(1-Boranotriphosphate), 2′-Deoxyguanosine-5′-O-(1-Boranotriphosphate), and 2′-Deoxythymidine-5′-O-(1-Boranotriphosphate).

In some embodiments, an mRNA may include the addition of a “cap” on the N-terminal (5′) end, and a “tail” on the C-terminal (3′) end. The presence of the cap may provide resistance to nucleases found in eukaryotic cells. The presence of a “tail” may protect the mRNA from exonuclease degradation.

### Cap Structure

In some embodiments, an mRNA may include a 5′ cap structure. A 5′ cap may comprise for example, a triphosphate linkage and a guanine nucleotide in which the 7-nitrogen is methylated. Examples of cap structures include, but are not limited to, m7G(5′)ppp(5′)A, G(5′)ppp(5′)A, and G(5′)ppp(5′)G. Naturally occurring cap structures comprise a 7-methyl guanosine that is linked via a triphosphate bridge to the 5′-end of the first transcribed nucleotide, resulting in a dinucleotide cap of m7G(5′)ppp(5′)N, where N is any nucleoside. In vivo, the cap is added in the nucleus by the enzyme guanylyl transferase immediately after initiation of transcription.

In some embodiments, a 5′cap may comprise an m7(3′OmeG)(5′)ppp(5′)(2′OmeA)pG or (CleanCap™ 3′ OMe) structure. In some embodiments, a 5′ cap may comprise a m7G(5′)ppp(5′)G. In some embodiments, the Anti-Reverse Cap Analog (“ARCA”) or modified ARCA, is a 5′ cap in which the 2′ or 3′ OH group is replaced with —OCH3. In some embodiments, the ARCA comprises an 3′-O-Me-m7G(5′)ppp(5′)G structure. In some embodiments, the 5′ cap comprises m7G(5′)ppp(5′)(2′OmeA)pG. Additional mRNA caps may include, but are not limited to, a chemical structures selected from the group consisting of m7GpppG, m7GpppA, m7GpppC; unmethylated caps (e.g., GpppG); a memethylated cap (e.g., m2′7GpppG), a trimethylated cap analog, or anti reverse cap analogs (e.g., ARCA; m7,2′0meGpppG, m72′dGpppG, m7′3′0meGpppG, m7,3 dGpppG and their tetraphosphate derivatives) (see, e.g., Jemielity, J. et al, ‘Wove anti-reverse cap analogs with superior translational properties”, RNA, 9:1108-1122 (2003)).

In some embodiments, a suitable cap is a 7-methyl guanylate (“m7G”) linked via a triphosphate bridge to the 5′-end of the first transcribed nucleotide, resulting in m7G(5′)ppp(5′)N, where N is any nucleoside. An embodiment of an m7G cap utilized in embodiments of the disclosure is m7G(5′)ppp(5′)G. In some embodiments, the cap is a Cap0 structure. Cap0 structures lack a 2′-O-methyl residue of the ribose attached to bases 1 and 2. In some embodiments, the cap is a Cap1 structure. Cap1 structures have a 2′-0-methyl residue at base 2. In some embodiments, the cap is a Cap2 structure. Cap2 structures have a 2′-0-methyl residue attached to both bases 2 and 3.

A variety of m7G cap analogs are known in the art, many of which are commercially available. These include the m7 GpppG described above, as well as the ARCA 3′-OCH3 and 2′-OCH3 cap analogs (Jemielity, J. et al., RNA, 9:1108-1122 (2003)). Additional cap analogs for use in embodiments of the disclosure include N7-benzylated dinucleoside tetraphosphate analogs (described in Grudzien, E. et at, RNA, 10:1479-1487 (2004)), phosphorothioate cap analogs (described in Grudzien-Nogalska, E., et al, RNA, 13:1745-1755 (2007)), and cap analogs (including biotinylated cap analogs) described in U.S. Pat. Nos. 8,093,367 and 8,304,529, incorporated by reference herein.

In some embodiments, the 5′ cap is inosine, N1-methyl-guanosine, 2′fluoro-guanosine, 7-deaza-guanosine, m7(3′OmeG)(5′)ppp(5′)(2′OmeA)pG, CleanCap™, m7(3′OmeG)(5′)ppp(5′)(2′OmeA)pG, 8-oxo-guanosine, 2-amino-guanosine, LNA-guanosine, 2-azido-guanosine, Cap2, Cap4, CAP-003, or CAP-225.

In some embodiments, the 5′ cap comprises or consists of an internal ribosome entry site (IRES). In some embodiments the IRES is within the 5′ UTR. In some embodiments, the 5′ cap comprises or consists of a 2A self-cleavage peptide, e.g, one or more of P2A, T2A, E2A and F2A.

### Tail Structure

The presence of a “tail” may serve to protect an mRNA from exonuclease degradation. The poly-A tail is thought to stabilize natural messengers and synthetic sense RNA. Therefore, in certain embodiments a long poly-A tail can be added to an mRNA molecule thus rendering the RNA more stable. Poly-A tails can be added using a variety of art-recognized techniques. For example, long poly-A tails can be added to synthetic or in vitro transcribed RNA using poly-A polymerase (Yokoe, et al. Nature Biotechnology. 1996; 14:1252-1256). A transcription vector can also encode long poly-A tails. In addition, poly-A tails can be added by transcription directly from PCR products. Poly-A may also be ligated to the 3′ end of a sense RNA with RNA ligase (see, e.g., Molecular Cloning A Laboratory Manual, 2^{nd} Ed., ed. By Sambrook, Fritsch and Maniatis (Cold Spring Harbor Laboratory Press: 1991 edition)).

In some embodiments, an mRNA may include a 3′ poly(A) tail structure. The length of the poly-A tail may be at least about 10, 50, 100, 200, 300, 400 or at least about 500 nucleotides. In some embodiments, a poly-A tail on the 3′ terminus of an mRNA may include about 10 to 300 adenosine nucleotides (e.g., about 10 to 200 adenosine nucleotides, about 10 to 150 adenosine nucleotides, about 10 to 100 adenosine nucleotides, about 20 to 70 adenosine nucleotides, or about 20 to 60 adenosine nucleotides). In some embodiments, the poly A tail is 120 adenosine nucleotides.

In some embodiments, an mRNA may include a 3′ poly-C tail structure. A poly-C tail on the 3′ terminus of mRNA may include about 10 to 200 cytosine nucleotides (e.g., about 10 to 150 cytosine nucleotides, about 10 to 100 cytosine nucleotides, about 20 to 70 cytosine nucleotides, about 20 to 60 cytosine nucleotides, or about 10 to 40 cytosine nucleotides). The poly-C tail may be added to the poly-A tail or may substitute the poly-A tail. In some embodiments, the length of the poly-A or poly C tail is associated with the stability of a modified sense mRNA and, therefore, the transcription of the protein. For example, because the length of the poly-A tail may influence the half-life of a sense mRNA molecule, the length of the poly-A tail may be adjusted to modify the level of resistance of the mRNA to nucleases, thereby providing more control over the time course of polynucleotide expression and/or polypeptide production.

### 5′ and 3′ Untranslated Regions (UTRs)

In some embodiments, an mRNA may include 5′ untranslated region (UTR) and/or a 3′ UTR. In some embodiments, a 5′ UTR may include one or more elements that affect the stability or translation of an mRNA. In some embodiments, the 5′UTR for example, may include an iron responsive element. In some embodiments, 5′ UTR may be between about 50 to about 100, or from about 50 to about 500 nucleotides in length. In some embodiments, 3′ UTR includes one or more of a poly-A signal, a binding site for proteins that may affect mRNA stability or localization, or one or more binding sites for miRNAs. In some embodiments, 3′ UTR may be between about 0 and about 50 nucleotides, or about 50 to about 100 nucleotides in length.

Example 3′ and 5′ UTR sequences may be derived from mRNAs with relatively long half-lives (e.g., globin, actin, GAPDH, tubulin, histone, or citric acid cycle enzymes) to increase the stability of the sense mRNA molecule. For example, 5′ UTR sequence may include a partial sequence of a cytomegalovirus (CMV) immediate-early 1 (IE1) gene, or a fragment thereof to improve the nuclease resistance and/or improve the half-life of the polynucleotide. In another example, the 5′ UTR could include the sequence of the tobacco etch virus (TEV). Generally, these modifications improve the stability and/or pharmacokinetic properties (e.g., half-life) of the polynucleotide relative to their unmodified counterparts, and include, for example modifications made to improve such polynucleotides resistance to in vivo nuclease digestion.

In some embodiments, a UTR may improve tissue specific expression, e.g., in the lung. In some embodiments, the 3′ UTR is a mouse alpha-globin 3′ UTR. In another example the 3′UTR could include the sequence of xenopus globin. In some embodiments, the UTR may be any of, or functional variants of, those described in any of PCT Application No. WO2017053297A1 and U.S. Pat. No. 10,519,189B2, both of which are incorporated herein in their entirety.

### Ionizable Lipids

In some embodiments of the disclosure, an LNP may comprise an ionizable lipid, e.g. SS-OP or analogs thereof, or MC3 or analogs thereof. The charge of the lipid may depend on pH of the surrounding solution, making it an ionizable lipid. The ionizable lipid may also be cleavable. The ionizable lipid may be cationic at ranges of pH found in endosomes or lysosomes in mammalian cells.

An ionizable lipid may refer to any of a number of lipid species that have a net positive charge at a selected pH, such as a physiological pH. In some embodiments, an LNP may comprise an ionizable lipid as disclosed in either of WO 2010/053572 or WO 2012/170930, or variations thereof, both of which are incorporated herein by reference in their entirety.

In some embodiments, an LNP may comprise one or more of MC3 (((6Z,9Z,28Z,31Z)-heptatriaconta-6,9,28,31-tetraen-19-yl 4-(dimethylamino)butanoate), DLin-MC3-DMA (4-(dimethylamino)-butanoic acid, (10Z,13Z)-1-(9Z,12Z)-9,12-octadecadien-1-yl-10,13-nonadecadien-1-yl ester), or analogs thereof including but not limited to LenMC3, γ-LenMC3, MC3MC, MC2C, MC2MC, MC3 Thioester, MC3 Ether, MC4 Ether, MC3 Alkyne, MC3 Amide, Pan-MC3, Pan-MC4, Pan-MC5, CP-LenMC3, CP-γ-LenMC3, CP-MC3, D-Lin-MC2-DMA, Lipid 5, SM-102, ALC-0315, and combinations thereof.

In some embodiments, an LNP may comprise one or more of cKK-E12 (3,6-Bis(4-(bis(2-hydroxydodecyl)amino)butyl)piperazine-2,5-dione), DLinDAP (1,2-dilineoyl-3-dimethylammonium-propane), DLin-DMA, DLin-D-DMA, DLin-KC2-DMA (2,2-dilinoleyl-4-dimethylaminoethyl-[1,3]-dioxolane), and DODMA.

In some embodiments, the ionizable lipid may comprise SS-OP or analogs thereof. In some embodiments, the ionizable lipid is a compound of Formula (1):

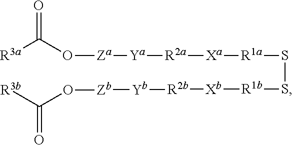

In the formula (1): R^{1a} and R^{1b} each independently represents an alkylene group having 1 to 6 carbon atoms, and may be linear or branched. The alkylene group may have 1 to 4 carbon atoms, or may have 1 to 2. Specific examples of the alkylene group having 1 to 6 carbon atoms include a methylene group, an ethylene group, a trimethylene group, an isopropylene group, a tetramethylene group, an isobutylene group, a pentamethylene group, and a neopentylene group. R^{1a} and R^{1b} may be each independently a methylene group, an ethylene group, a trimethylene group, an isopropylene group, or a tetramethylene group, and may be an ethylene group.

R^{1a} may be different or be the same as R^{1b}.

X^{a} and X^{b} are each independently an acyclic alkyl tertiary amino group having 1 to 6 carbon atoms and 1 tertiary amino group, or 2 to 5 carbon atoms, and a cyclic alkylene tertiary amino group having 1 to 2 tertiary amino groups, and/or each independently a cyclic alkylene having 2 to 5 carbon atoms and 1 to 2 tertiary amino groups and an alkylene tertiary amino group.

The alkyl group having 1 to 6 carbon atoms in the acyclic alkyl tertiary amino group having 1 to 6 carbon atoms and 1 tertiary amino group is branched even if it is linear. The alkyl group may be annular. The alkyl group may have 1 to 3 carbon atoms. Specific examples of the alkyl group having 1 to 6 carbon atoms include methyl group, ethyl group, propyl group, isopropyl group, n-butyl group, sec-butyl group, isobutyl group, tert-butyl group, pentyl group, and isopentyl group. Neopentyl group, t-pentyl group, 1,2-dimethylpropyl group, 2-methylbutyl group, 2-methylpentyl group, 3-methylpentyl group, 2,2-dimethylbutyl group, 2,3-dimethylbutyl group, A cyclohexyl group etc. can be mentioned.

A specific structure of an acyclic alkyl tertiary amino group having 1 to 6 carbon atoms and 1 tertiary amino group is represented by X¹.

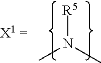

R⁵ of X¹ represents an alkyl group having 1 to 6 carbon atoms and may be linear, branched or cyclic. The alkyl group may have 1 to 3 carbon atoms. Specific examples of the alkyl group having 1 to 6 carbon atoms include methyl group, ethyl group, propyl group, isopropyl group, n-butyl group, sec-butyl group, isobutyl group, tert-butyl group, pentyl group, and isopentyl group. Neopentyl group, t-pentyl group, 1,2-dimethylpropyl group, 2-methylbutyl group, 2-methylpentyl group, 3-methylpentyl group, 2,2-dimethylbutyl 2 group, 2,3-dimethylbutyl group, A cyclohexyl group etc. can be mentioned.

The number of carbon atoms in the cyclic alkylene tertiary amino group having 2 to 5 carbon atoms and 1 to 2 tertiary amino groups may be 4 to 5. Specific examples of the cyclic alkylene tertiary amino group having 2 to 5 carbon atoms and 1 to 2 tertiary amino groups include aziridylene group, azetidylene group, pyrrolidylene group, piperidylene group, imidazolidylene group, a piperazylene group, optionally a pyrrolidylene group, a piperidylene group or a piperazylene group.

Number is 2 to 5 carbon atoms, and specific structure of alkylene tertiary amino groups containing 1 annular tertiary amino group represented by X².

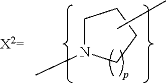

P of X² is 1 or 2. When p is 1, X² is a pyrrolidylene group, and when p is 2, X² is a piperidylene group.

A specific structure of a cyclic alkylene tertiary amino group having 2 to 5 carbon atoms and 2 tertiary amino groups is represented by X³.

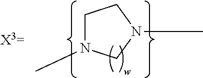

W of X³ is 1 or 2. When w is 1, X³ is an imidazolidylene group, and when w is 2, X³ is a piperazylene group.

X^{a} may be different be identical to X^{b}.

R^{2a} and R^{2b} each independently represent an alkylene group or an oxydialkylene group having 8 or less carbon atoms, optionally each independently an alkylene group having 8 or less carbon atoms.

The alkylene group having 8 or less carbon atoms may be linear or branched but is optionally linear. The number of carbon atoms contained in the alkylene group is optionally 6 or less, and optionally 4 or less. Specific examples of the alkylene group having 8 or less carbon atoms include methylene group, ethylene group, propylene group, isopropylene group, tetramethylene group, isobutylene group, pentamethylene group, hexamethylene group, heptamethylene group, octamethylene group, and the like. In some embodiments included are a methylene group, an ethylene group, a propylene group, and a tetramethylene group.

The oxydialkylene group having 8 or less carbon atoms refers to an alkylene group (alkylene-O-alkylene) via an ether bond, and the total number of carbon atoms of two alkylene groups is 8 or less. Here, the two alkylenes may be the same or different, but are optionally the same. Specific examples of the oxydialkylene group having 8 or less carbon atoms include an oxydimethylene group, an oxydiethylene group, an oxydipropylene group, and an oxydibutylene group.

R^{2a} may be same or different and R^{2b}.

Y^{a} and Y^{b} are each independently an ester bond, an amide bond, a carbamate bond, an ether bond or a urea bond, optionally each independently an ester bond, an amide bond or a carbamate bond. While Y binding orientation of Y^{a} and Y^{b} are not limited, if Y^{a} and Y^{b} is an ester bond, optionally, —Z^{a}—CO—R^{2a}— and —Z^{b}—CO—O—R²-Structure.

Y^{a} may be different or identical to Y^{b}.

Z^{a} and Z^{b} are each independently a divalent group derived from an aromatic compound having 3 to 16 carbon atoms, having at least one aromatic ring, and optionally having a hetero atom. Represents. The number of carbon atoms contained in the aromatic compound is optionally 6 to 12, or 6 to 7. Moreover, the number of aromatic rings contained in the aromatic compound is optionally one.

As the types of aromatic rings contained in the aromatic compound having 3 to 16 carbon atoms, as for aromatic hydrocarbon rings, benzene ring, naphthalene ring, anthracene ring, and aromatic heterocycles as imidazole ring, pyrazole ring, oxazole ring, Isoxazole ring, thiazole ring, isothiazole ring, triazine ring, pyrrole ring, furanthiophene ring, pyrimidine ring, pyridazine ring, pyrazine ring, pyridine ring, purine ring, pteridine ring, benzimidazole ring, indole ring, benzofuran ring, quinazoline ring, phthalazine ring, quinoline ring, isoquinoline ring, coumarin ring, chromone ring, benzodiazepine ring, phenoxazine ring, phenothiazine ring, acridine ring, etc., optionally benzene ring, naphthalene ring, anthracene ring.

The aromatic ring may have a substituent. Examples of the substituent include an acyl group having 2 to 4 carbon atoms, an alkoxycarbonyl group having 2 to 4 carbon atoms, a carbamoyl group having 2 to 4 carbon atoms, and 2 to 2 carbon atoms. 4 acyloxy groups, acylamino groups having 2 to 4 carbon atoms, alkoxycarbonylamino groups having 2 to 4 carbon atoms, fluorine atoms, chlorine atoms, bromine atoms, iodine atoms, alkylsulfanyl groups having 1 to 4 carbon atoms, 1 carbon atom Alkylsulfonyl group having 4 to 4, arylsulfonyl group having 6 to 10 carbon atoms, nitro group, trifluoromethyl group, cyano group, alkyl group having 1 to 4 carbon atoms, ureido group having 1 to 4 carbon atoms, 1 to carbon atoms 4 alkoxy groups, aryl groups having 6 to 10 carbon atoms, aryloxy groups having 6 to 10 carbon atoms, and the like. Some examples include acetyl groups, methoxycarbonyl groups, methyl carbonate groups, and the like, moyl group, acetoxy group, acetamide group, methoxycarbonylamino group, fluorine atom, chlorine atom, bromine atom, iodine atom, methylsulfanyl group, phenylsulfonyl group, nitro group, trifluoromethyl group, cyano group, methyl group, ethyl group Propyl group, isopropyl group, t-butyl group, ureido group, methoxy group, ethoxy group, propoxy group, isopropoxy group, t-butoxy group, phenyl group and phenoxy group.

A specific structure of Z^{a} and Z^{b} includes Z¹.

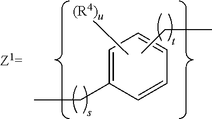

Wherein, s represents an integer of 0 to 3, t represents an integer of 0 to 3, u represents an integer of 0 to 4, represents a u-number of R 4 is independently a substituent.

S in Z¹ is optionally an integer of 0 to 1.

T in Z¹ is optionally an integer of 0 to 2.

U in Z¹ is optionally an integer of 0 to 2.

R 4 in Z¹ is a substituent of an aromatic ring (benzene ring) contained in an aromatic compound having 3 to 16 carbon atoms that does not inhibit the reaction in the process of synthesizing the ionizable lipid. Examples of the substituent include an acyl group having 2 to 4 carbon atoms, an alkoxycarbonyl group having 2 to 4 carbon atoms, a carbamoyl group having 2 to 4 carbon atoms, an acyloxy group having 2 to 4 carbon atoms, and an acylamino group having 2 to 4 carbon atoms, an alkoxycarbonylamino group having 2 to 4 carbon atoms, fluorine atom, chlorine atom, bromine atom, iodine atom, alkylsulfanyl group having 1 to 4 carbon atoms, alkylsulfonyl group having 1 to 4 carbon atoms, 6 to 10 carbon atoms Arylsulfonyl group, nitro group, trifluoromethyl group, cyano group, alkyl group having 1 to 4 carbon atoms, ureido group having 1 to 4 carbon atoms, alkoxy group having 1 to 4 carbon atoms, aryl group having 6 to 10 carbon atoms And aryloxy groups having 6 to 10 carbon atoms, and examples include acetyl, methoxycarbonyl, methylcarbamoyl, acetoxy, Mido group, methoxycarbonylamino group, fluorine atom, chlorine atom, bromine atom, iodine atom, methylsulfanyl group, phenylsulfonyl group, nitro group, trifluoromethyl group, cyano group, methyl group, ethyl group, propyl group, isopropyl group, T-butyl group, ureido group, methoxy group, ethoxy group, propoxy group, isopropoxy group, t-butoxy group, phenyl group and phenoxy group. When a plurality of R⁴ are present, each R⁴ may be the same or different.

Z^{a} may be different even identical to the Z^{b}.

R^{3a} and R^{3b} are each independently a residue derived from a reaction product of a fat-soluble vitamin having a hydroxyl group and succinic anhydride or glutaric anhydride, or a sterol derivative having a hydroxyl group and succinic anhydride or glutaric acid. Represents a residue derived from a reaction product with an anhydride, or an aliphatic hydrocarbon group having 12 to 22 carbon atoms, and optionally each independently a fat-soluble vitamin having a hydroxyl group and succinic anhydride or glutaric anhydride. Or a C 12-22 aliphatic hydrocarbon group, and optionally each independently an aliphatic hydrocarbon group having 12-22 carbon atoms.

Examples of the fat-soluble vitamin having a hydroxyl group include retinol, ergosterol, 7-dehydrocholesterol, calciferol, corcalciferol, dihydroergocalciferol, dihydrotaxolol, tocopherol, and tocotrienol. The fat-soluble vitamin having a hydroxyl group is optionally tocopherol.

Examples of the sterol derivative having a hydroxyl group include cholesterol, cholestanol, stigmasterol, β-sitosterol, lanosterol, ergosterol and the like, optionally cholesterol or cholestanol.

The aliphatic hydrocarbon group having 12 to 22 carbon atoms may be linear or branched. The aliphatic hydrocarbon group may be saturated or unsaturated. In the case of an unsaturated aliphatic hydrocarbon group, the number of unsaturated bonds contained in the aliphatic hydrocarbon group is usually 1 to 6, optionally 1 to 3, or 1 to 2. Unsaturated bonds include carbon-carbon double bonds and carbon-carbon triple bonds. The number of carbon atoms contained in the aliphatic hydrocarbon group is optionally 13 to 19, or 13 to 17. The aliphatic hydrocarbon group includes an alkyl group, an alkenyl group, an alkynyl group and the like, and optionally includes an alkyl group or an alkenyl group. Specific examples of the aliphatic hydrocarbon group having 12 to 22 carbon atoms include dodecyl, tridecyl, tetradecyl, pentadecyl, hexadecyl, heptadecyl, octadecyl, nonadecyl, icosyl, heicosyl, docosyl, Dodecenyl group, tridecenyl group, tetradecenyl group, pentadecenyl group, hexadecenyl group, heptadecenyl group, octadecenyl group, nonadecenyl group, icocenyl group, henicocenyl group, dococenyl group, dodecadienyl group, tridecadienyl group, tetradecadienyl group, pentadecadienyl group Group, hexadecadienyl group, heptadecadienyl group, octadecadienyl group, nonadecadienyl group, icosadenyl group, henicosadienyl group, docosadienyl group, octadecatrienyl group, icosatrienyl group, Cosatetraenyl group, icosapentaenyl group, docosahexaenyl group, isostearyl group, 1-hexylheptyl group, 1-hexylnonyl group, 1-octylnonyl group, 1-octylundecyl group, 1-decylundecyl group, etc. be able to. The aliphatic hydrocarbon group having 12 to 22 carbon atoms is optionally a tridecyl group, a pentadecyl group, a heptadecyl group, a nonadecyl group, a heptadecenyl group, a heptadecadienyl group, or a 1-hexylnonyl group, or a tridecyl group, A heptadecyl group, a heptadecenyl group, and a heptadecadienyl group.

In one embodiment of the present disclosure, the aliphatic hydrocarbon group having 12 to 22 carbon atoms represented by R^{3a} and R^{3b} is derived from a fatty acid. In this case, the carbonyl carbon derived from the fatty acid is contained in —CO—O— in the formula (1). Specific examples of the aliphatic hydrocarbon group include a heptadecenyl group when linoleic acid is used as the fatty acid, and a heptadecenyl group when oleic acid is used as the fatty acid.

R^{3a} may be different be the same as R^{3b}.

In one embodiment of the present disclosure, R^{1a} is the same as R^{1b}, X^{a} is the same as X^{b}, R^{2a} is the same as R^{2b}, Y^{a} is the same as Y^{b}, and Z^{a} is identical to the Z^{b}, R^{3a} is the same as R^{3b}.

Preferable examples of the ionizable lipid represented by the formula (1) include the following ionizable lipids:Ionizable lipid (1-1); R1a and R1b are each independently an alkylene group having 1 to 6 carbon atoms (e.g., methylene group, ethylene group); X a and X b are each independently an acyclic alkyl tertiary amino group having 1 to 6 carbon atoms and 1 tertiary amino group (e.g., —N(CH3)-), Or a cyclic alkylene tertiary amino group having 2 to 5 carbon atoms and 1 to 2 tertiary amino groups (e.g., piperidylene group); R^{2a} and R^{2b} are each independently an alkylene group having 8 or less carbon atoms (e.g., methylene group, ethylene group, propylene group); Y^{a} and Y^{b} are each independently an ester bond or an amide bond; Z^{a} and Z^{b} are each independently a divalent group derived from an aromatic compound having 3 to 16 carbon atoms, having at least one aromatic ring, and optionally having a hetero atom. (E.g., —C6H4-CH2-, —CH2-C6H4-CH2-); R^{3a} and R^{3b} are each independently a residue derived from a reaction product of a fat-soluble vitamin having a hydroxyl group (e.g., tocopherol) and succinic anhydride or glutaric anhydride, or an aliphatic group having 12 to 22 carbon atoms A hydrocarbon group (e.g., heptadecenyl group, heptadecadienyl group, 1-hexylnonyl group);

Ionizable lipid (1-2); R^{1a} and R^{1b} are each independently an alkylene group having 1 to 4 carbon atoms (e.g., methylene group, ethylene group); X a and X b are each independently an acyclic alkyl tertiary amino group having 1 to 3 carbon atoms and 1 tertiary amino group (e.g., —N(CH3)-), Or a cyclic alkylene tertiary amino group having 2 to 5 carbon atoms and 1 tertiary amino group (e.g., piperidylene group); R^{2a} and R^{2b} are each independently an alkylene group having 6 or less carbon atoms (e.g., methylene group, ethylene group, propylene group); Y^{a} and Y^{b} are each independently an ester bond or an amide bond; Za and Zb are each independently a divalent group derived from an aromatic compound having 6 to 12 carbon atoms, one aromatic ring, and optionally having a hetero atom (e.g., —C6H4-CH2-, —CH2-C6H4-CH2-); R^{3a} and R^{3b} are each independently a residue derived from a reaction product of a fat-soluble vitamin having a hydroxyl group (e.g., tocopherol) and succinic anhydride, or an aliphatic hydrocarbon group having 13 to 19 carbon atoms (e.g., Heptadecenyl group, heptadecadienyl group, 1-hexylnonyl group).

Ionizable lipid (1-3); R^{1a} and R^{1b} are each independently an alkylene group having 1 to 2 carbon atoms (e.g., methylene group, ethylene group); X^{a} and X^{b} are each independently X¹:

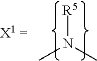

wherein R⁵ is an alkyl group having 1 to 3 carbon atoms (e.g., a methyl group)), or X²:

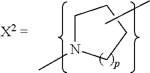

wherein p is 1 or 2), R^{2a} and R^{2b} are each independently an alkylene group having 4 or less carbon atoms (e.g., methylene group, ethylene group, propylene group); Y^{a} and Y^{b} are each independently an ester bond or an amide bond; Z^{a} and Z^{b} are each independently Z¹:

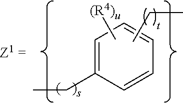

wherein s is an integer from 0 to 1, t is an integer from 0 to 2, u is an integer from 0 to 2 (optionally 0), and (R⁴)u are each independently represents a substituent. R^{3a} and R^{3b} are each independently a residue derived from a reaction product of a fat-soluble vitamin having a hydroxyl group (e.g., tocopherol) and succinic anhydride, or an aliphatic hydrocarbon group having 13 to 17 carbon atoms (e.g., Heptadecenyl group, heptadecadienyl group, 1-hexylnonyl group);

Specific examples of the ionizable lipid according to Formula 1 of the present disclosure include the following O-Ph-P3C1, O-Ph-P4C1, O-Ph-P4C2, O-Bn-P4C2, E-Ph-P4C2, L-Ph-P4C2, HD-Ph-P4C2, O-Ph-amide-P4C2, and O-Ph-C3M as seen in the following charts.

### Ionizable Lipids

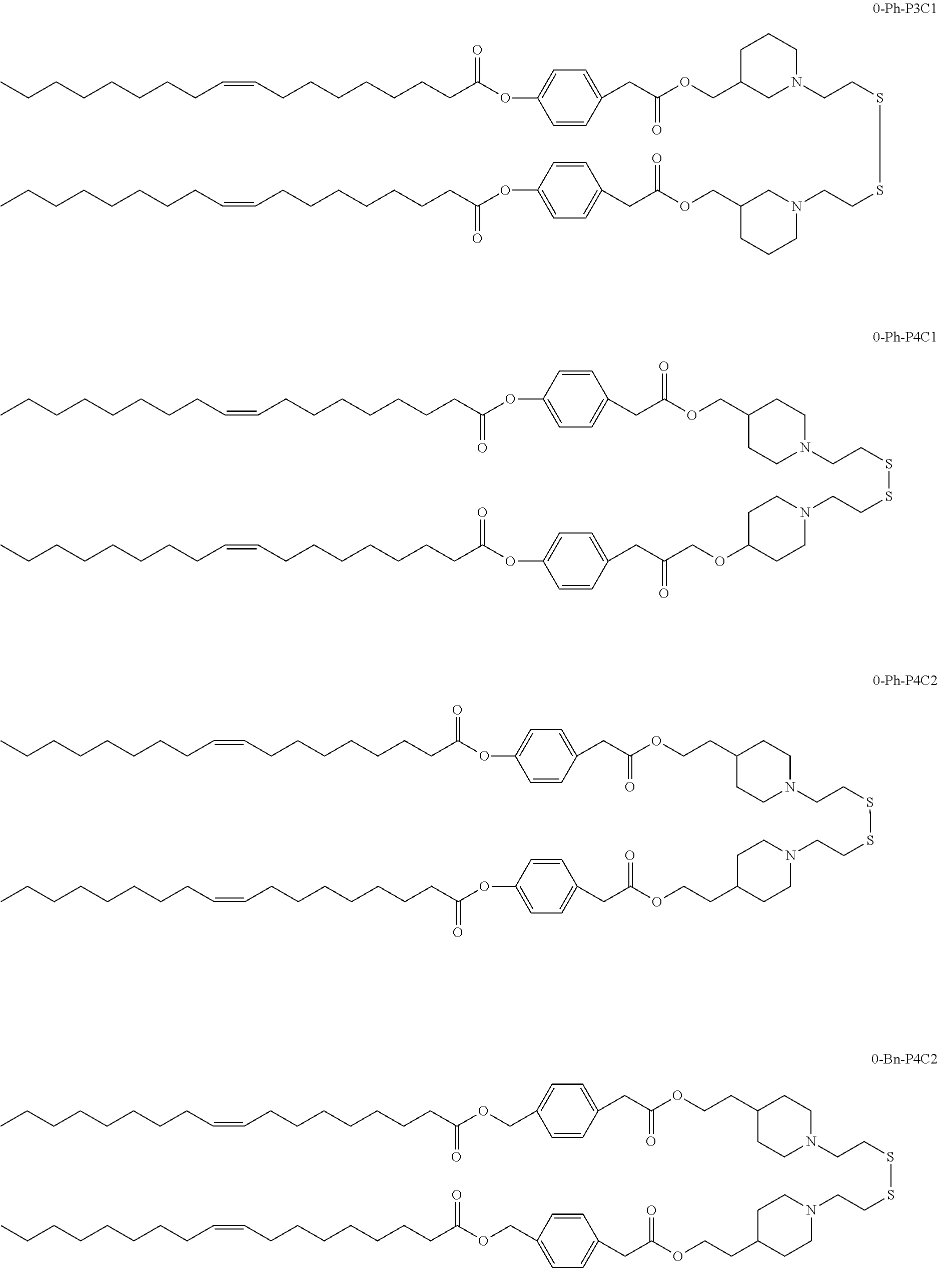 [SMILES: CCCCCCCC/C=C\CCCCCCCC(=O)Oc1ccc(CC(=O)OCC2CCCN(CCSSCCN3CCCC(COC(=O)Cc4ccc(OC(=O)CCCCCCC/C=C\CCCCCCCC)cc4)C3)C2)cc1 ; CCCCCCCC/C=C\CCCCCCCC(=O)Oc1ccc(CC(=O)COC2CCN(CCSSCCN3CCC(COC(=O)Cc4ccc(OC(=O)CCCCCCC/C=C\CCCCCCCC)cc4)CC3)CC2)cc1 ; CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1 ; CCCCCCCC/C=C\CCCCCCCC(=O)OCc1ccc(CC(=O)OCCC2CCN(CCSSCCN3CCC(CCOC(=O)Cc4ccc(COC(=O)CCCCCCC/C=C\CCCCCCCC)cc4)CC3)CC2)cc1]

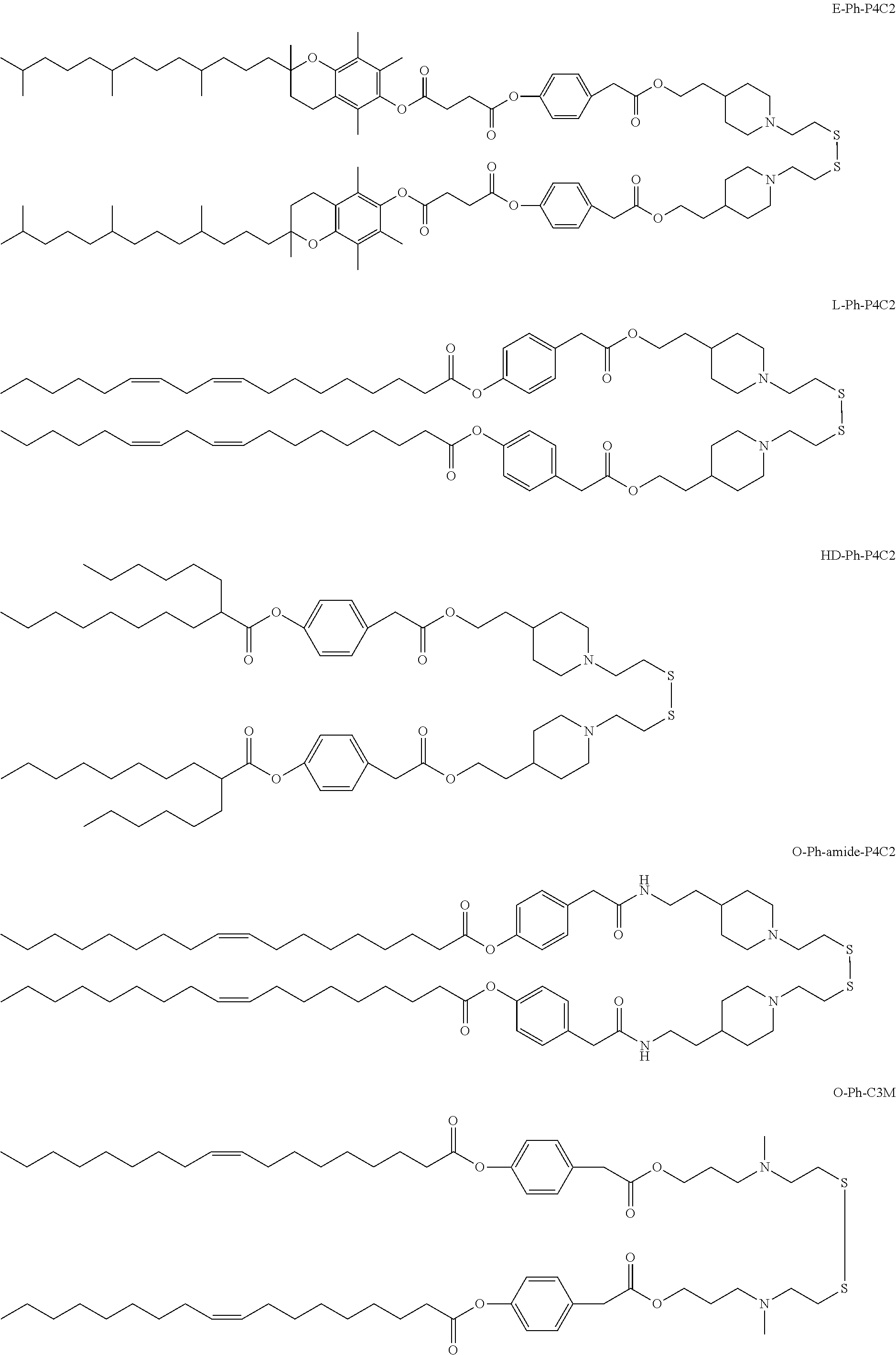

### Ionizable Lipids

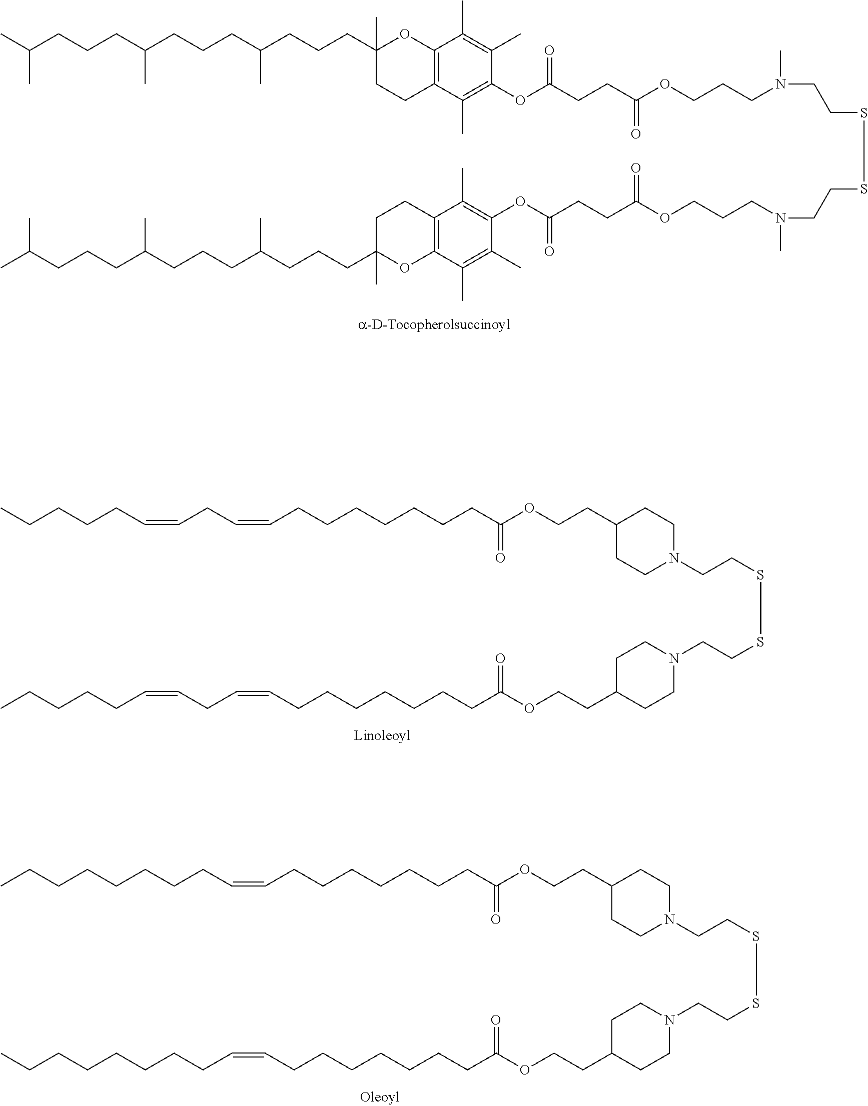 [SMILES: Cc1c(C)c2c(c(C)c1OC(=O)CCC(=O)OCCCN(C)CCSSCCN(C)CCCOC(=O)CCC(=O)Oc1c(C)c(C)c3c(c1C)CCC(C)(CCCC(C)CCCC(C)CCCC(C)C)O3)CCC(C)(CCCC(C)CCCC(C)CCCC(C)C)O2 ; CCCCC/C=C\C/C=C\CCCCCCCC(=O)OCCC1CCN(CCSSCCN2CCC(CCOC(=O)CCCCCCC/C=C\C/C=C\CCCCC)CC2)CC1 ; CCCCCCCC/C=C\CCCCCCCC(=O)OCCC1CCN(CCSSCCN2CCC(CCOC(=O)CCCCCCC/C=C\CCCCCCCC)CC2)CC1]

Lipids having the structure of Formula I are shown in the chart below. For example, SS-OP is also named 0-Ph-P4C2. The term “SS-OP analog” as used herein refers to a compound of Formula I.

### Nomenclature of Lipids

| Name | Structure |
|---|---|
| SS- M | 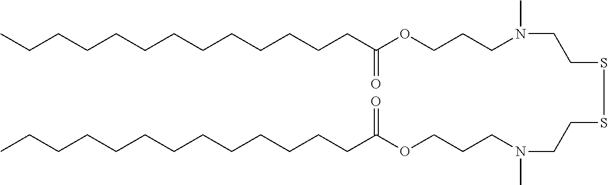 [SMILES: CCCCCCCCCCCCCC(=O)OCCCN(C)CCSSCCN(C)CCCOC(=O)CCCCCCCCCCCCC] |
| SS- E | 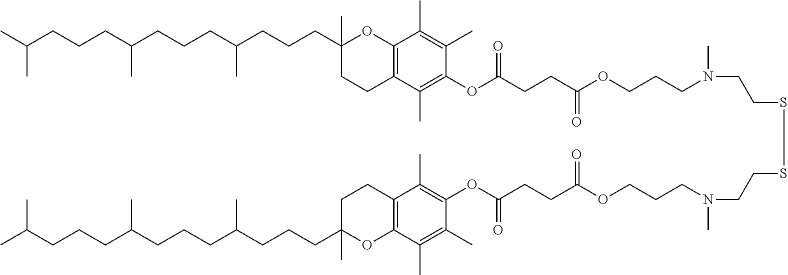 [SMILES: Cc1c(C)c2c(c(C)c1OC(=O)CCC(=O)OCCCN(C)CCSSCCN(C)CCCOC(=O)CCC(=O)Oc1c(C)c(C)c3c(c1C)CCC(C)(CCCC(C)CCCC(C)CCCC(C)C)O3)CCC(C)(CCCC(C)CCCC(C)CCCC(C)C)O2] |
| SS- EC | 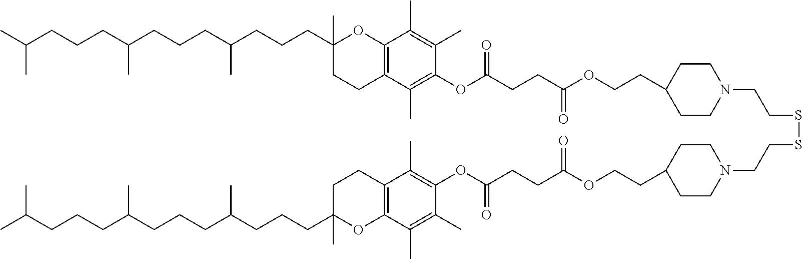 [SMILES: Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2] |
| SS- LC | 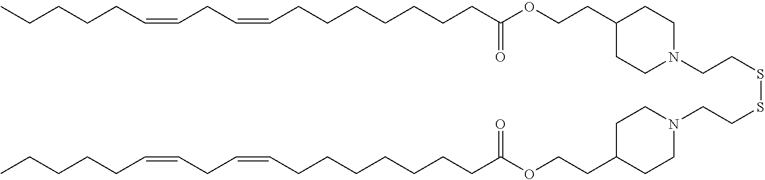 [SMILES: CCCCC/C=C\C/C=C\CCCCCCCC(=O)OCCC1CCN(CCSSCCN2CCC(CCOC(=O)CCCCCCC/C=C\C/C=C\CCCCC)CC2)CC1] |
| SS- OC | 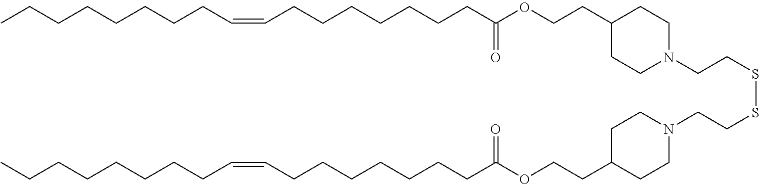 [SMILES: CCCCCCCC/C=C\CCCCCCCC(=O)OCCC1CCN(CCSSCCN2CCC(CCOC(=O)CCCCCCC/C=C\CCCCCCCC)CC2)CC1] |
| SS- OP | 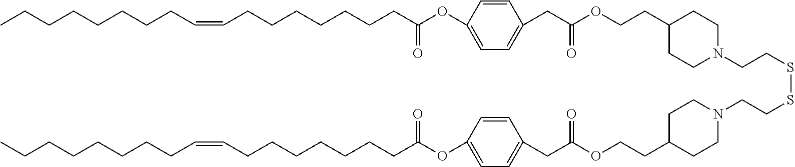 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1] |

### Cationic Lipids

In some embodiments, an LNP of the disclosure comprises a cationic lipid, e.g. DOTAP or variations thereof. The cationic lipid may be a “permanent cationic lipid.” The term cationic lipid may be cationic in pH ranges found in mammalian physiological environments such as blood or interstitial fluids. Cationic lipids may be composed of a cationic amine moiety and a lipid moiety, and the cationic amine moiety and a polyanion nucleic acid may interact to form a positively charged liposome or lipid membrane structure. Thus, uptake into cells may be promoted and nucleic acids delivered into cells.

In some embodiments, the cationic lipid may selected from one or more of 1,2-dioleoyl-3-trimethylammonium-propane (DOTAP), N,N-distearyl-N,N-dimethylarnmonium bromide (DABB), or 1,2-dimyristoyl-sn-glycero-3-ethylphosphocholine (EPC). In some embodiments, an LNP comprises a ionizable lipid wherein the ionizable lipid is one or more of N-[1-(2,3-dioleyloxy) propyl]-N,N,N-trimethylammonium chloride (DOTMA), 5-carboxyspermylglycinedioctadecylamide (DOGS), 2,3-dioleyloxy-N-[2 (spermine-carboxamido)ethyl]-N,N-dimethyl-1-propanaminium (DOSPA), 1,2-Dioleoyl-3-Dimethylammonium-Propane (DODAP), 11,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), dimethyldioctadecylammonium (DDA), 1,2-dilinolenyloxy-N,N-dimethyl-3-aminopropane (DLenDMA), N-dioleyl-N,N-dimethylammonium chloride (DODAC), N,N-distearyl-N,N-dimethylammonium bromide (DDAB), N-(1,2-dimyristyloxyprop-3-yl)-N,N-dimethyl-N-hydroxyethyl ammonium bromide (DMRIE), 3-dimethylamino-2-(cholest-5-en-3-beta-oxybutan-4-oxy)-1-(cis, cis-9,12-octadecadienoxy)propane (CLinDMA), 2-[5′-(cholest-5-en-3-beta-oxy)-3′-oxapentoxy)-3-dimethy 1-1-(cis,cis-9′,1-2′-octadecadienoxy) propane (CpLinDMA), N,N-dimethyl-3,4-dioleyloxy benzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane or (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), and mixtures thereof.

In some embodiments, a cationic lipid refers to a cationic cholesterol lipid. In some embodiments of the disclosure an LNP comprises imidazole cholesterol ester (ICE). In some embodiments, an ICE structure is substantially similar to:

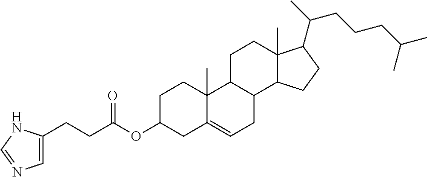 [SMILES: CC(C)CCCC(C)C1CCC2C3CC=C4CC(OC(=O)CCc5cnc[nH]5)CCC4(C)C3CCC12C]

In some embodiments of the disclosure an LNP comprises 25-Hydroxycholesterol (25 OH Chol). In some embodiments, 25 OH Chol structure is substantially similar to:

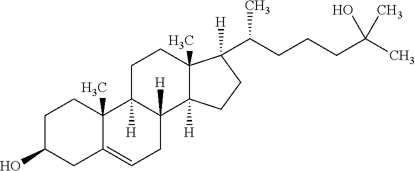

In some embodiments of the disclosure an LNP comprises 20α-hydroxycholesterol 5-cholestene-3a.

In some embodiments, the 20α-hydroxycholesterol 5-cholestene-3a (also known as 20α-diol or 20a chol structure) is substantially similar to:

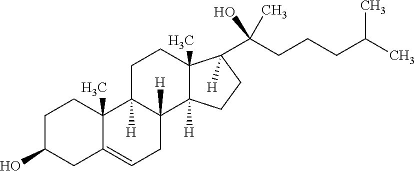

In some embodiments, a cationic lipid refers to dimethyldioctadecylammonium bromide (DDAB). In some embodiments of the disclosure an LNP comprises dimethyldioctadecylammonium bromide (DDAB). In some embodiments, a dimethyldioctadecylammonium bromide (DDAB) structure is substantially similar to:

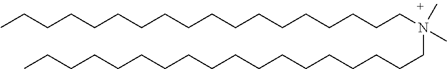

### Structural Lipids

In some embodiments, the LNP comprises a structural lipid. As used herein, structural lipids are lipids that contribute a physical or chemical property to the LNP that is in addition to, or independent of, electrical charge. As an example, structural lipids may tend to have a shape, size, rigidity, hydrophobicity, or other property that increases the therapeutic utility of the LNP, such as, for example, by increasing its stability, half-life, deformability, transfection efficiency, tropism, thermostability, resistance to aggregation, membrane fluidity, or other parameter. In some embodiments, structural lipids are neutral in charge, either due to lacking charged moieties, or due to being zwitterionic with balanced charges summing to zero net charge.

In some embodiments, an LNP may comprise a structural lipid selected from one more of: 1,2-di-(9Z-octadecenoyl)-sn-glycero-3-phosphoethanolamine (DOPE), glycerol-monooleate (GMO), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), or variants thereof.

In some embodiments, an LNP may include one or more phosphatidyl lipids, for example, the phosphatidyl compounds (e.g., phosphatidylglycerol, phosphatidylcholine, phosphatidylserine and phosphatidylethanolamine). In some embodiments, an LNP may comprise sphingolipids, for example but not limited to, sphingosine, ceramide, sphingomyelin, cerebroside and ganglioside. In some embodiments, the aforementioned “structural” lipids contribute to the stability and/or specificity of the LNP composition.

### Cholesterol-Based Lipids

In some embodiments, an LNP may comprise one or more cholesterol-based lipids. A cholesterol-based lipid may include but is not limited to: PEGylated cholesterol, DC-Choi (N,N-dimethyl-N-ethylcarboxamidocholesterol), 1,4-bis(3-N-oleylamino-propyl)piperazine.

### PEGylated Lipids

In some embodiments of the disclosure, an LNP may comprise one or more PEGylated lipids. For example, the use of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000] (C8 PEG-2000 ceramide) is contemplated by the present disclosure in combination with one or more of the ionizable and/or other lipids. In some embodiments, PEGylated lipids comprise PEG-ceramides having shorter acyl chains (e.g., C14 or C18). In some embodiments, the PEGylated lipid DSPE-PEG-Maleimide-Lectin may be used. Other contemplated PEG-modified lipids include, but are not limited to, a polyethylene glycol chain of up to 5 kDa in length covalently attached to a lipid with alkyl chain(s) of C6-C2o length. Without wishing to be bound by a particular theory, it is contemplated that the addition of PEGylated lipids may prevent complex aggregation and increase circulation lifetime to facilitate the delivery of the liposome encapsulated mRNA to the target cell.

### Methods of Treatment

Methods of treatment as described herein refer to extending telomeres in the lung in a subject in need thereof by administration of an LNP of the present disclosure comprising one or more mRNA sequences. Compositions and methods of the disclosure may be used for the treatment of conditions in or involving lung or lung cells. In some embodiments, compositions and/or methods of use of compositions of the disclosure intended for treatment of fibrotic conditions, including fibrosis. In some embodiments, compositions and/or methods of use of compositions of the disclosure intended for treatment of fibrotic conditions, including fibrosis, induce encapsulated mRNA expression in lung tissue. In some embodiments, compositions and/or methods of use of compositions of the disclosure intended for treatment do not induce cellular, tissue or systemic toxicity. Compositions may be administered systemically, e.g., intravenously. In some embodiments, compositions and/or methods of use of compositions of the disclosure intended for treatment of conditions in or involving lung tissue cells, do not induce cellular or systemic toxicity. Compositions may be administered systemically, e.g., intravenously.

In the compositions and methods described herein, in some embodiments, an LNP pharmaceutical composition contemplated herein is administered in a dose of about 0.001 mg/kg per the subject's body weight to about 2.0 mg/kg per the subject's body weight to a subject in need thereof. In some embodiments, a Targeting LNP is administered to a subject in need thereof in a dose of about 0.01 mg/kg; in some embodiments in a dose of about 0.025 mg/kg; in some embodiments in a dose of about 0.05 mg/kg; in some embodiments in a dose of about 0.075 mg/kg; in some embodiments in a dose of about 0.1 mg/kg; in some embodiments in a dose of about 0.125 mg/kg; in some embodiments in a dose of about 0.150 mg/kg; in some embodiments in a dose of about 0.175 mg/kg; in some embodiments in a dose of about 0.2 mg/kg; in some embodiments in a dose of about 0.5 mg/kg; in some embodiments in a dose of about 0.75 mg/kg; in some embodiments in a dose of about 1.0 mg/kg; in some embodiments, in a dose of about 1.25 mg/kg; in some embodiment in a dose of about 1.5 mg/kg; or in some embodiment in a dose of about 2.0 mg/kg. In some embodiments the LNP is administered to a subject in need thereof in a dose of 0.1 mg/kg. In some embodiments the LNP is administered to a subject in need thereof in a dose of 0.125 mg/kg.

In some embodiments LNP pharmaceutical compositions contemplated herein are administered to a subject in need thereof in a single dose. In some embodiments the LNP is administered to a subject in need thereof two, three, four, or five or more times. In some embodiments, the LNP is administered twice a week, every week, every two weeks, every four weeks, every six weeks, every twelve weeks, or every fifteen weeks. In some embodiments, the LNP is administered every month, every two months, every three months, every six months, once a year, on an ongoing basis, or as determined by their physician.

In some embodiments, the contemplated LNP pharmaceutical compositions are delivered in an aerosolized inhaled form, orally, subcutaneously, intravenously, intranasally, intradermally, transdermally, intraperitoneally, intramuscularly, intrapulmonarily, vaginally, rectally, or intraocularly. In example embodiments targeting LNPs are administered intravenously.

In some embodiments, an exemplary LNP pharmaceutical composition includes an excipient, or carrier, e.g., an aqueous carrier. A variety of aqueous carriers can be used, e.g., buffered saline. The compositions may contain pharmaceutically acceptable auxiliary substances as those required to approximate physiological conditions such as pH and buffering agents, toxicity countering agents, e.g., sodium acetate, sodium chloride, sodium citrate, potassium chloride, calcium chloride, and sodium lactate. In some embodiments, the pharmaceutical composition comprises 10 mM sodium citrate buffered to pH 6.4. The composition may contain a cryoprotectant, e.g., glycerol, ethylene glycol, sucrose, propylene glycol, or dimethylsulfoxide (DMSO). The concentration of active agent in these formulations can vary and are selected based on fluid volumes, viscosities, and body weight in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)).

In other embodiments, the compounds or compositions are administered without isolating the cell or cells, the tissue, or the organ from the subject (i.e., the administration is in vivo). In some of these embodiments, the compound or composition is delivered to all, or almost all, cells in the subject's body. In some embodiments, the compound or composition is delivered to a specific cell, cell type, tissue, or organ in the subject's body.

Administration of the compounds or compositions of the instant disclosure may result in the transient expression of an mRNA within the LNP in the cell. The increased activity may be measured by various assays, such as, for example, the telomerase repeat amplification protocol (TRAP) assay. Commercial versions of the TRAP assay are available, for example the Trapeze® telomerase detection kit (Millipore), which provides a sensitive detection and quantitation of telomerase activity, although other measurement techniques are also possible.

As previously noted, one of the advantages of the instant techniques is that the expression of mRNA in the LNP is transient in the transfected cells. In particular, such transient expression is in contrast to previous techniques where, for example, a telomerase reverse transcriptase gene persists in an episomal DNA moiety, or is inserted into the genomic sequence of the cell or otherwise permanently modifies the genetic make-up of the targeted cell and results in constitutive activity of the nucleic acid sequence.

In some embodiments of the instant methods, the transient expression is independent of cell cycle.

### Therapeutic Kits

Therapeutic kits comprising a pharmaceutical composition of an LNP contemplated herein, or lipid components thereof provided in separate containers, and instructions for making and/or using are also contemplated herein. In some embodiments, the therapeutic kit comprises devices for administration, including but not limited to syringes, inhalers, nebulizers, and vials or containers. In some embodiments, components of an exemplary therapeutic kit are provided such that they can be mixed with commercially available microfluidic or vortex mixers. Often such kits are provided with or without nucleic acid (e.g., mRNA) that is to be encapsulated in the LNPs made with the components of the therapeutic kit.

In another aspect, the instant disclosure provides ready-to-use kits for use in extending telomeres in a mammalian cell. The kits comprise any of the above-described compounds or compositions, together with instructions for their use. In some embodiments, the kits further comprise packaging materials. In some embodiments, the packaging materials are air-tight. In these embodiments, the packaging materials may optionally be filled with an inert gas, such as, for example, nitrogen, argon, or the like. In some embodiments, the packaging materials comprise a metal foil container, such as, for example, a sealed aluminum pouch or the like. Such packaging materials are well known by those of ordinary skill in the art. The kit may also comprise a delivery vehicle, such as a lipid as described herein. In some embodiments, one or more components of the formulation are provided frozen with a cryoprotectant, or lyophilized.

In some embodiments, the kit may further comprise a desiccant, a culture medium, an RNase inhibitor, or other such components. In some embodiments, the kit may further comprise a combination of more than one of these additional components. In some kit embodiments, the composition of the kit is sterile.

### EMBODIMENTS

In embodiment 1, a method of delivering a polynucleotide to a subject is provided, comprising administering, by intravenous injection, a polynucleotide encapsulated in a lipid nanoparticle (LNP) comprising: a cationic lipid in a molar percentage of between about 25% and about 60%, an ionizable lipid to cationic lipid ratio of between 0.7 and 1.8, and an ionizable lipid comprising either: (a) SS-OP or an SS-OP analog at a molar percentage of between about 25% and about 60%, wherein the LNP has a lipid nitrogen:polynucleotide phosphate ratio (N/P ratio) of between 10 and 30, or (b) DLin-MC3-DMA, or a DLin-MC3-DMA analog, at a molar percentage of between at or about 30% and at or about 50%, wherein the LNP has an N/P ratio of between 3.5 and 10.

In embodiment 2, the method of embodiment 1 is provided, wherein the polynucleotide comprises a synthetic RNA, which upon or after administration of the LNP, the mRNA is translated into a corresponding protein encoded by the mRNA in vivo in the subject, and the half-life of the lipid DOTAP in vivo in the: a) lung of the subject is at least 30 hours; b) liver of the subject is no more than 11 hours; and/or c) spleen of the subject is no more than 9 hours.

In embodiment 3, the method of embodiment 2 is provided, wherein the half-life of the protein in vivo in the lung of the subject is at least 10 hours, 15 hours, 20 hours, 25 hours, 30 hours, or 35 hours.

In embodiment 4, the method of any of embodiments 1 to 3 is provided, wherein the polynucleotide comprises a synthetic RNA, which upon or after administration of the LNP, the mRNA is translated into a corresponding protein encoded by the mRNA in vivo in the subject, and wherein at or about 6 hours after intravenous infusion of the LNP, the lung:liver ratio of the amount of the protein per mg of tissue is at least 2:1, 3:1, or 4:1.

In embodiment 5, the method of any of embodiments 1 to 4 is provided, wherein the lung:liver ratio of the amount of the protein per mg of tissue is at least 5:1, 6:1, 7:1, 8:1, 9:1, 10:1, 20:1, or 50:1.

In embodiment 6, the method of any of embodiments 1 to 5 is provided, wherein the polynucleotide comprises a synthetic RNA encoding a telomerase reverse transcriptase (TERT), which upon or after administration of the LNP, the mRNA is translated into a corresponding TERT protein in vivo in the subject.

In embodiment 7, the method of any of embodiments 1 to 6 is provided, wherein the median telomere length of lung alveolar cells is extended by at least 5%, at least 10%, at least 20%, or at least 50%.

In embodiment 8, the method of any of embodiments 1 to 7 is provided, wherein the median telomere length of lung AT2 cells is extended by at least 5%, at least 10%, at least 20%, or at least 50%.

In embodiment 9, the method of any of embodiments 1 to 8 is provided, wherein the increase in telomerase activity in the lung is detectable by the TRAP assay for at least 24 or 48 hours.

In embodiment 10, the method of any of embodiments 1 to 9 is provided, wherein the LNP has an N/P ratio of between 28 and 31.

In embodiment 11, the method of embodiment 10 is provided, wherein the LNP has an N/P ratio of at or about 29.1.

In embodiment 12, the method of any of embodiments 1 to 9 is provided, wherein the LNP has an N/P ratio of between 11 and 13.

In embodiment 13, the method of embodiment 12 is provided, wherein the LNP has an N/P ratio of at or about 12 or at or about 12.1.

In embodiment 14, the method of any of embodiments 1 to 9 is provided, wherein the LNP has an N/P ratio of between 4 and 5.

In embodiment 15, the method of embodiment 14 is provided, wherein the LNP has an N/P ratio of at or about 4.5.

In embodiment 16, the method of any of embodiments 1 to 11 is provided, wherein the ionizable lipid is SS-OP and the LNP has a cationic lipid to ionizable lipid ratio at or about 1.8 and is composed of 5 lipids.

In embodiment 17, the method of any of embodiments 1 to 11 is provided, wherein the ionizable lipid is SS-OP and the LNP has a cationic lipid to ionizable lipid ratio at or about 1.8 and is composed of 5 lipids, including DOTAP and DOPC.

In embodiment 18, the method of any of embodiments 1 to 11 is provided, wherein the ionizable lipid is SS-OP and the LNP has a cationic lipid to ionizable lipid ratio at or about 1.8 and is composed of 5 lipids, including DMG-PEG2000, DOPC and DOTAP.

In embodiment 19, the method of any of embodiments 1 to 9 or 12-13 is provided, wherein the ionizable lipid is SS-OP and the LNP has a cationic lipid to ionizable lipid ratio at or about 0.8 and is composed of 3 or 5 lipids.

In embodiment 20, the method of any of embodiments 1 to 9 or 12-13 is provided, wherein the ionizable lipid is SS-OP and the LNP has a cationic lipid to ionizable lipid ratio at or about 0.8 and is composed of 3 or 5 lipids, including DOTAP.

In embodiment 21, the method of any of embodiments 1 to 9 or 12-13 is provided, wherein the ionizable lipid is SS-OP and the LNP has a cationic lipid to ionizable lipid ratio at or about 0.8 and is composed of 3 or 5 lipids, including DMG-PEG2000 and DOTAP.

In embodiment 22, the method of any of embodiments 1 to 9 or 14-15 is provided, wherein the ionizable lipid is DLin-MC3-DMA and the LNP has a cationic lipid to ionizable lipid ratio at or about 1.5 and is composed of 3 lipids.

In embodiment 23, the method of any of embodiments 1 to 9 or 14-15 is provided, wherein the ionizable lipid is DLin-MC3-DMA and the LNP has a cationic lipid to ionizable lipid ratio at or about 1.5 and is composed of 3 lipids, including DOTAP.

In embodiment 24, the method of any of embodiments 1 to 9 or 14-15 is provided, wherein the ionizable lipid is DLin-MC3-DMA and the LNP has a cationic lipid to ionizable lipid ratio at or about 1.5 and is composed of 3 lipids, including DMG-PEG2000 and DOTAP.

In embodiment 25, the method of any of embodiments 1 to 9 or 14-15 is provided, wherein the ionizable lipid is DLin-MC3-DMA and the LNP has a cationic lipid to ionizable lipid ratio at or about 1.5 and is composed of 3 lipids, including DMG-PEG2000 and DOTAP, and N/P ratio is about 4.5.

In embodiment 26, the method of any one of claims 1 to 15 is provided, wherein the LNP is selected from an LNP of Table 8.

In embodiment 27, the LNP of any of embodiments 1 through 26 comprises a nucleic acid such as a ribonucleic acid or a deoxyribonucleic acid.

In embodiment 28, the ribonucleic acid of embodiment 27 comprises TERT mRNA.

In embodiment 29, the ribonucleic acid of any of embodiments 27 to 28 is transiently expressed in a cell.

In embodiment 30, the cell of embodiment 29 comprises a lung tissue cell.

In embodiment 31, the lung tissue cell of embodiment 30 comprises a lung alveolar cell such as an ST2 cell.

In embodiment 32, a LNP used in any of embodiments 1 through 31 is provided.

In embodiment 33, a 2-lipid LNP comprising DOTAP and DMG-PEG2000 and having a N/P ratio of at or about 4 is provided for use in any of embodiments 1 through 31.

In embodiment 34, a cell is provided comprising the LNP of any one of embodiments 1 to 33.

In embodiment 35, a kit comprising the components of the LNP of any of embodiments 1 to 34 is provided.

In embodiment 36, a kit comprising the LNP of any of embodiments 1 to 34 is provided.

In embodiment 37, the kit of embodiments 35 or 36 is provided, wherein the kit further comprises a nucleic acid such as a ribonucleic acid or a deoxyribonucleic acid.

In embodiment 38, a method of manufacturing the LNP of, used in, or formed by the components of, any of embodiments 1 through 37 is provided.

In embodiment 39, the method of manufacturing of embodiment 38 is provided, wherein the LNP is formed prior to introducing a ribonucleic acid or the ribonucleic acid to a solution containing the LNP.

### EXAMPLES

### Example 1

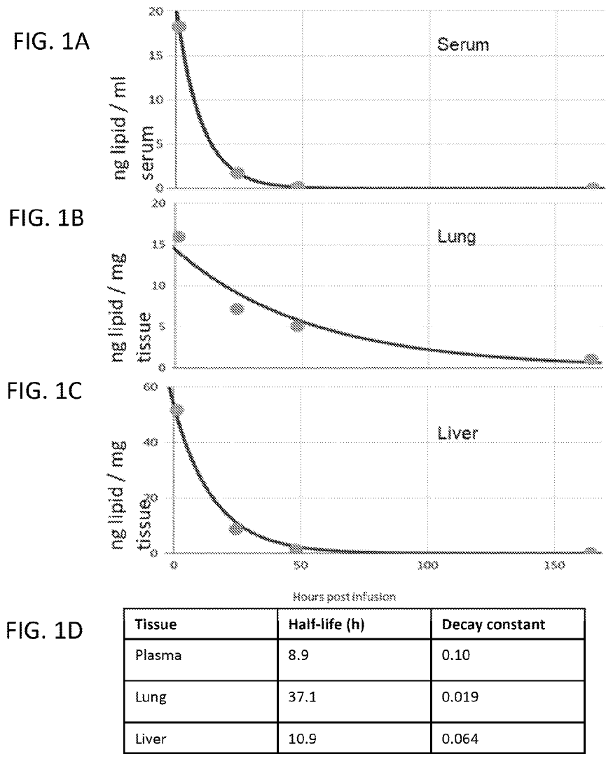

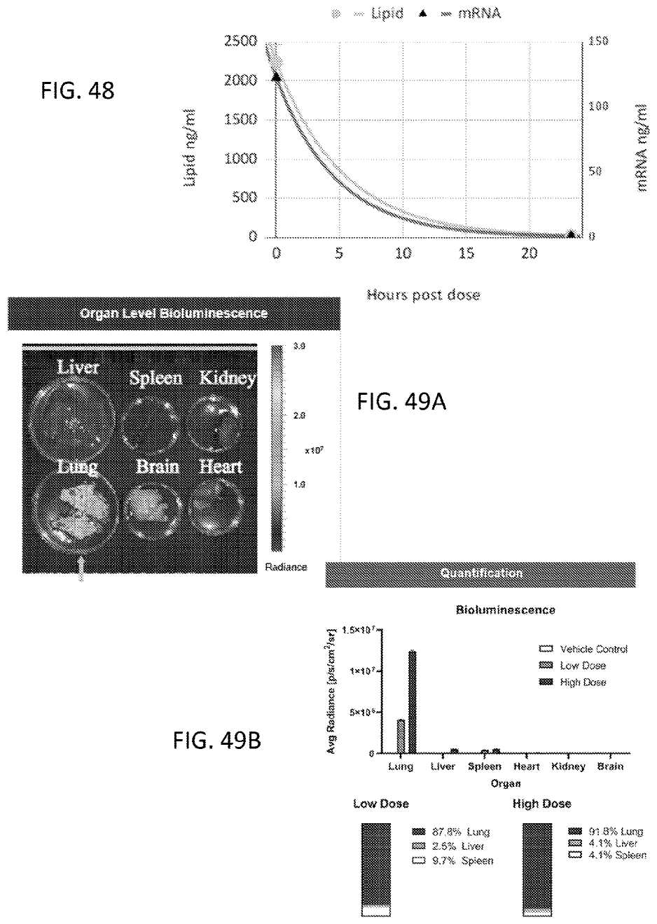

FIGS. 1 and 48 depict exemplary pharmacokinetics of the 5 lipid LNPs of Table 1. Timeline of plasma, lung, and liver levels of the 5 lipid LNPs as determined by measuring one of the lipids, DOTAP, are presented. In the process of degenerating these data, LNPs were formulated with TERT mRNA (Human TERT opti—SEQ ID NO: 1) and the lipids in the ratios of Table 1. Male C57Bl/6 mice were dosed at 1 mg/kg and plasma and tissues were collected at the time points indicated above. DOTAP was measured in the plasma, lung, and liver using LC-MS/MS. The nitrogen:phosphorus N/P charge ratio of the composition of Table 1 is 12.

TABLE 1

| Compound | Molar Ratio |
|---|---|
| SS-OP | 55 |
| DOPC | 5 |
| Cholesterol | 40 |
| DMG-PEG2000 | 2.5 |
| DOTAP | 44 |

N/P ratio (nitrogen:phosphorus charge ratio): 12

### Example 2

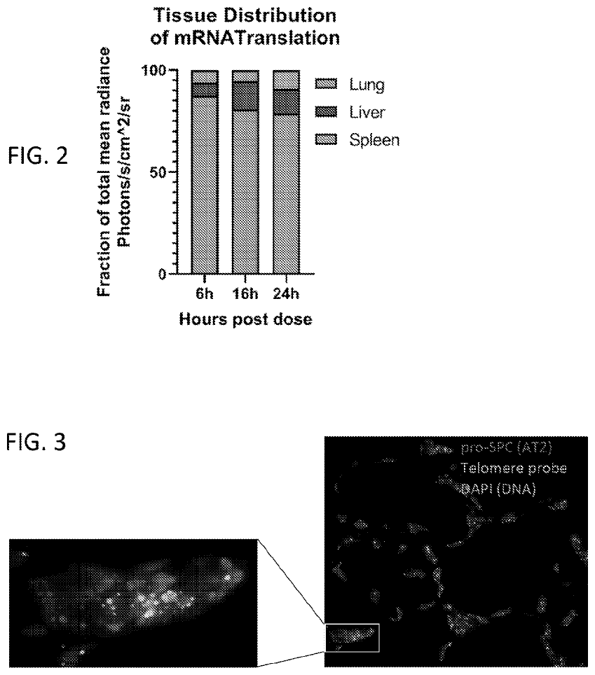

FIG. 2 depicts the ratio of bioluminescence signal from the indicated organs, produced from luciferase protein translated from mRNA injected intravenously, at the indicated time points post intravenous injection of the 5 lipid LNP of Table 2. In this process, LNPs were formulated with firefly luciferase mRNA and the lipids in the ratios of Table 2. Male C57B16 mice were dosed at 1.3 mg/kg and tissues were collected and imaged ex vivo at the time points indicated above. Mean radiance values were baseline normalized by subtracting the mean radiance from the negative control (no LNP) treated animal. The relative radiance between lung, liver, and spleen are plotted in FIG. 2. The tissues were also homogenized and the bioluminescence was measured from equal amounts by weight of homogenate. The nitrogen:phosphorus N/P charge ratio of the composition of Table 2 is 12.

TABLE 2

| Compound | Molar Ratio |
|---|---|
| SS-OP | 55 |
| DOPC | 5 |
| Cholesterol | 40 |
| DMG-PEG2000 | 2.5 |
| DOTAP | 44 |

N/P ratio (nitrogen:phosphorus charge ratio): 12

### Example 3

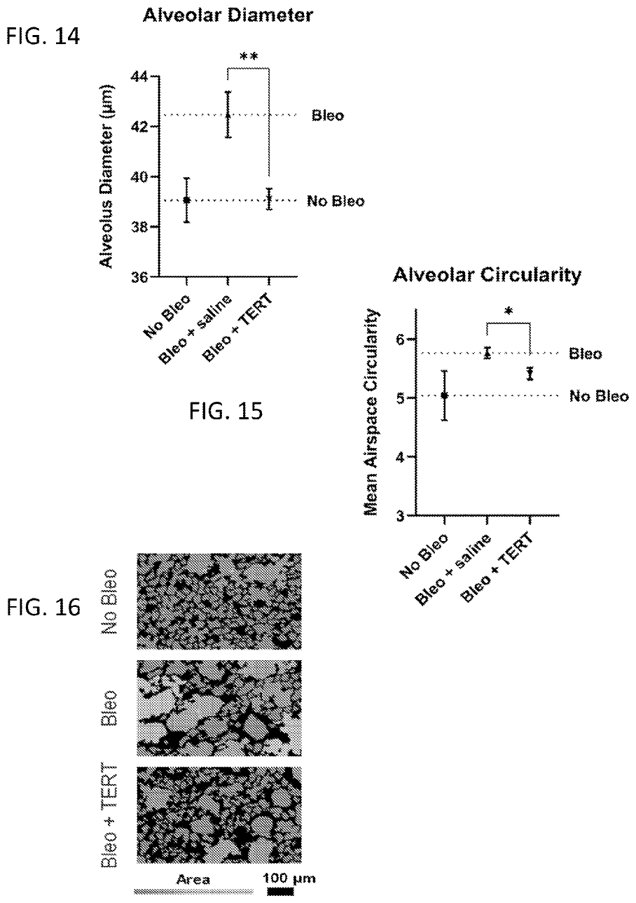

FIGS. 3-17 and FIGS. 20-21 depict experiments involving the use of a different 5 lipid LNP composition where lung fibrosis was induced in third generation (G3) TERT knock-out (KO) mice, which have telomere lengths that are similar to those of humans. Bleomycin was administered via oropharyngeal aspiration at 2.0 U/kg. For FIGS. 13-16, an initial dose of bleomycin was administered via oropharyngeal aspiration at 0.5 U/kg one week prior to the 2.0 U/kg dose. LNPs were formulated with firefly luciferase mRNA (Ctrl) or TERT mRNA (mouse SEQ ID NO: 2, including synthetic 5′ UTR (SEQ ID NO: 3), wt mTert coding sequence (SEQ ID NO: 4) and mouse alpha-globin 3′ UTR (SEQ ID NO: 5)) (referred to as “TERT mRNA LNP” herein or “TERT” or “TERT mRNA” in the figures) and the lipids in the ratios of Table 3. The DOTAP molar ratio is 2.27 times higher in the 5 lipid LNP of Table 3. Mice were dosed at 1.5 mg/kg twice a week for two weeks beginning on day 7 post bleomycin. Some mice received bleomycin, but not LNP treatment (No LNP), and control mice received saline instead of bleomycin and LNP treatment (No Bleo). Tissues were harvested on day 24 post bleomycin. Lungs were paraffin fixed, formalin embedded. To measure telomere length, sections were stained using the Q-FISH method with the telomere probe and antibody against pro-surfactant protein C (SPC) to label the alveolar type II (AT2) cells. Telomerase activity was measured in lung tissue lysate using the telomeric repeat amplification protocol (TRAP) assay.

TABLE 3

| Compound | Molar Ratio |
|---|---|
| SS-OP | 55 |
| DOPC | 5 |
| Cholesterol | 40 |
| DMG-PEG2000 | 2.5 |
| DOTAP | 100 |

N/P ratio (nitrogen:phosphorus charge ratio): 26.5

FIG. 3 depicts a representative immunofluorescence micrograph of a lung section used to measure telomere length by the Q-FISH method known in the field, in which the intensity of a telomere probe is proportional to telomere length, and the cells were co-stained with an antibody to the AT2 cell marker pro-SPC.

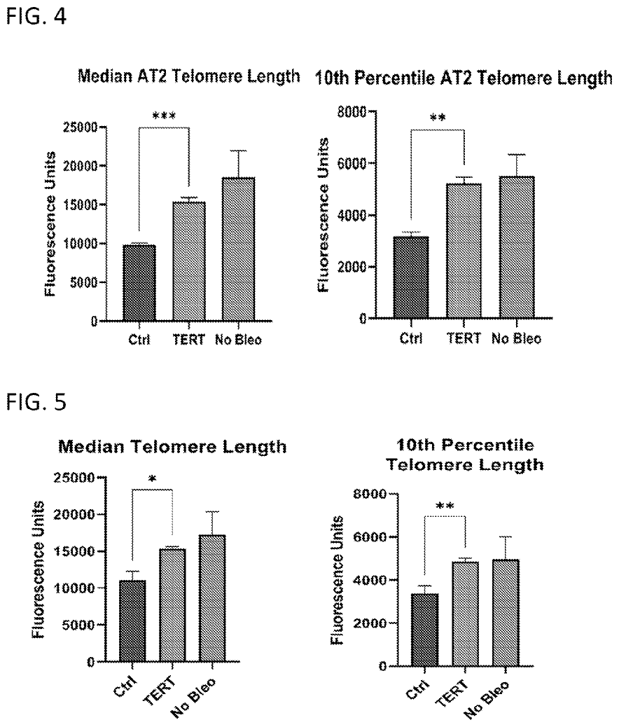

FIG. 4 depicts measurements of telomere length by Q-FISH in AT2 cells in mice in which fibrosis had been induced using bleomycin (“Bleo”) as described above. Treatment with TERT mRNA LNPs (“TERT” in the figure) of Table 3 increased the median telomere length and 10^{th} percentile telomere length in AT2 cells (cells that were scored as pro-SPC+ by immunofluorescence staining) as measured by Q-FISH, relative to mice receiving luciferase mRNA LNPs (referred to as “Ctrl” in the figure), almost to the length found in normal mice that had not received bleomycin (“No Bleo” in the figure).

FIG. 5 depicts measurements of telomere length by Q-FISH in AT2 cells in mice in which fibrosis had been induced using bleomycin (“Bleo”) as described above. Treatment with TERT mRNA LNPs (“TERT” in the figure) of Table 3 increased the median telomere length and 10^{th} percentile telomere length in lung alveolar cells as measured by Q-FISH, relative to mice receiving luciferase mRNA LNPs (referred to as “Ctrl” in the figure), almost to the length found in normal mice that had not received bleomycin (“No Bleo” in the figure). Fields of view that were predominantly filled with alveoli were used for the quantification, and telomere lengths of all cell types present were included. Typically, alveolar tissue includes epithelial, endothelial, fibroblasts, immune cells, and other cell types.

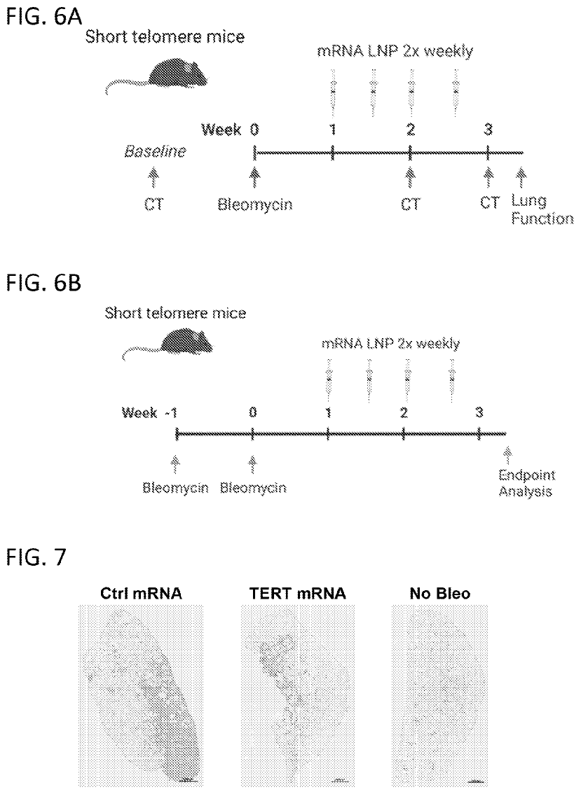

FIG. 6A depicts the timeline of the bleomycin mouse experiment described above. “CT” indicates x-ray computed tomography scanning of the mice to quantify normo-aerative lung volume.

FIG. 6B depicts the timeline of the bleomycin mouse experiment for FIGS. 13-16.

FIG. 7 depicts false color images of lung sections stained with Sirius Red for quantification of fibrotic foci and fibrosis by area of tissue covered with collagen deposition. IV infusion of TERT mRNA LNPs of Table 3 reduced fibrosis by 62%.

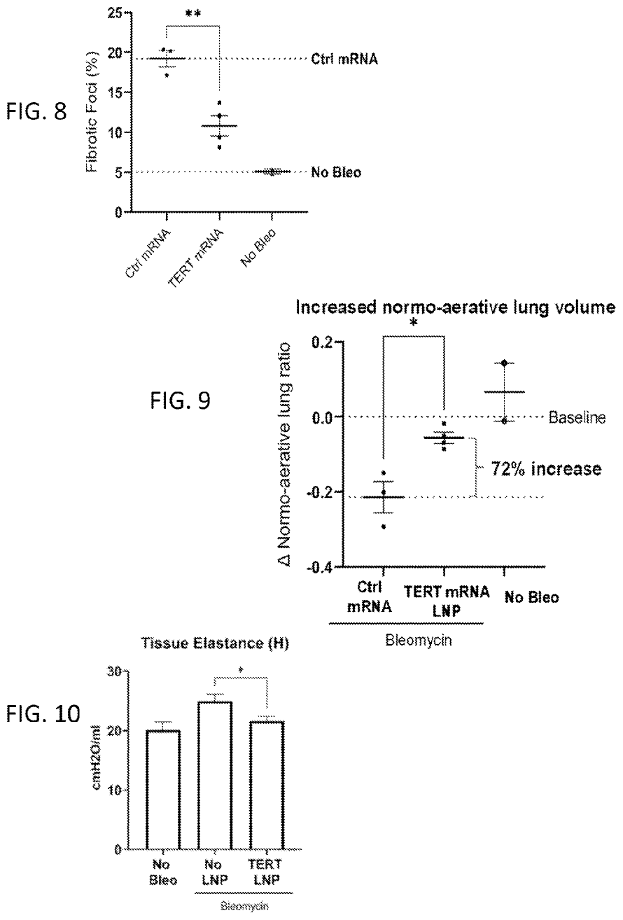

FIG. 8 depicts reduction of fibrotic foci by TERT mRNA LNP of Table 3 treatment in bleomycin-treated mice as measured by Sirius Red staining.

FIG. 9 depicts increased normo-aerative lung ratio, or amount of useful lung volume, by TERT mRNA LNP of Table 3 treatment in bleomycin-treated mice as measured by x-ray computed tomography imaging followed by quantification of fibrotic tissue based on x-ray intensity of the voxels.

FIG. 10 depicts improvement of lung elastance by TERT mRNA LNP of Table 3 treatment in bleomycin-treated mice as measured by the Flexivent system.

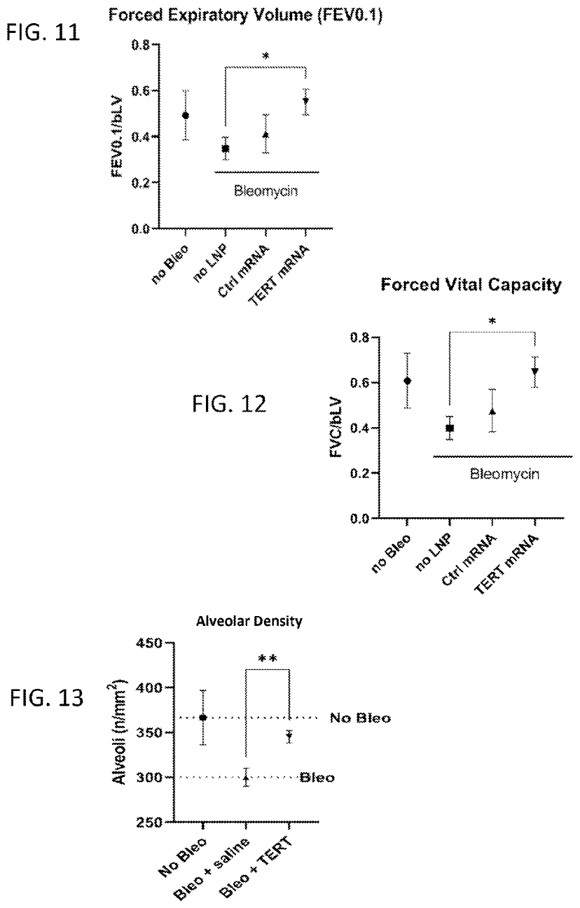

FIGS. 11 and 12 depict improvement of lung forced expiratory volume (FEV0.1) and forced vital capacity (FVC), respectively, by TERT mRNA LNP of Table 3 treatment in bleomycin-treated mice as measured by the Flexivent system.

FIGS. 13, 14, and 15 depicts improvement in alveolar density, alveolar diameter, and alveolar circularity, respectively, by TERT mRNA LNP of Table 3 treatment in bleomycin-treated mice as measured by machine vision analysis of micrographs of lung sections by the company Biocellvia.

FIG. 16 depicts examples of false color images produced by Biocellvia of lung tissue sections during their analysis of alveolar architecture quantified in FIGS. 13-15.

FIG. 17 depicts measurements of telomerase activity in isolated primary human alveolar epithelial cells from a 50 year-old donor at the indicated time points after treatment with TERT mRNA LNPs of Table 3 at a concentration of 500 ng/ml as measured using the telomeric repeat amplification protocol (TRAP) assay.

FIG. 18 depicts the timeline of an experiment to measure the effect of TERT mRNA LNP of Table 3 treatment on the colony formation capacity of isolated primary human alveolar epithelial cells from a 50 year-old donor. The cells were plated in medium that supports colony formation, treated with TERT mRNA LNPs at a concentration of 500 ng/ml of TERT mRNA, and incubated for 7 days after which colonies were counted, as reported in FIG. 19.

FIG. 19 depicts an increase in colony formation capacity of human alveolar epithelial cells from a 50 year-old donor by TERT mRNA LNP of Table 3 treatment.

### Example 4—Reduction in Senescence after Telomerase mRNA LNP Treatment in Mice

Methods: The experiment was performed as described in connection with FIGS. 3-12.

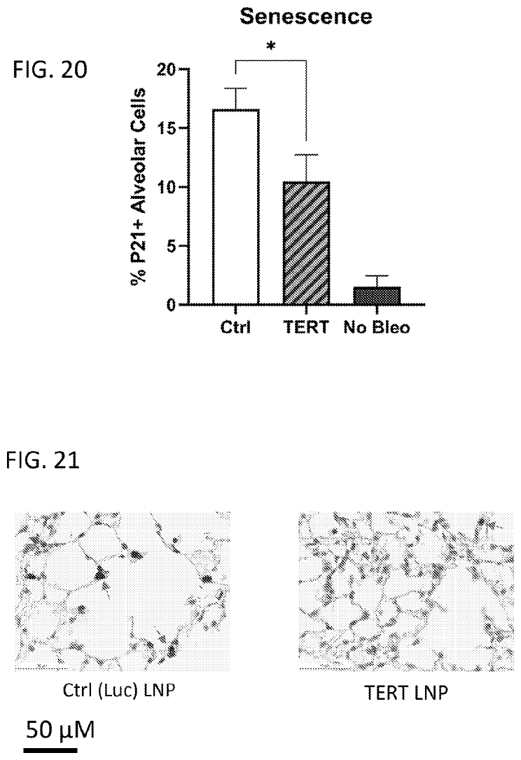

Results: As shown in FIGS. 20-21, a 40% reduction in senescence marker P21 was shown in the lung alveolar cells of mice treated with telomerase mRNA LNP compared to mice treated with luciferase mRNA LNP (Ctrl). In FIG. 21, dark cells are positive. Increase in senescence following bleomycin treatment.

### Example 5—Efficient Lung Delivery Using 2 Lipid LNPs

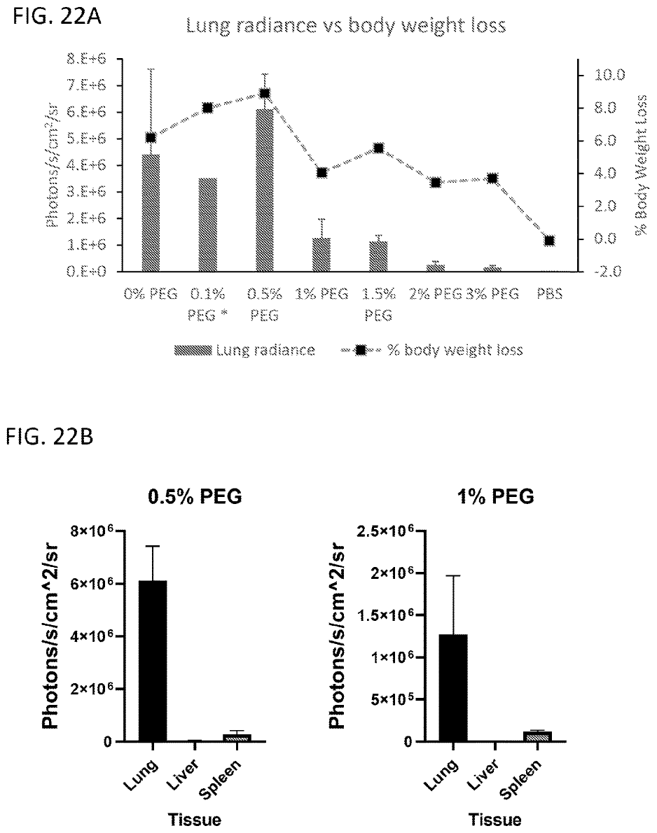

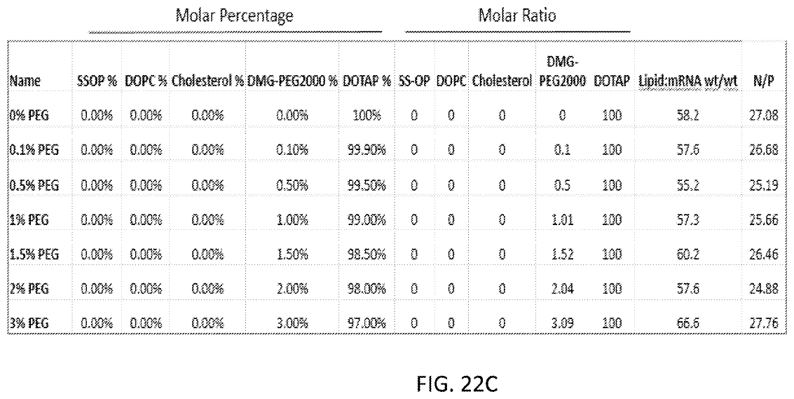

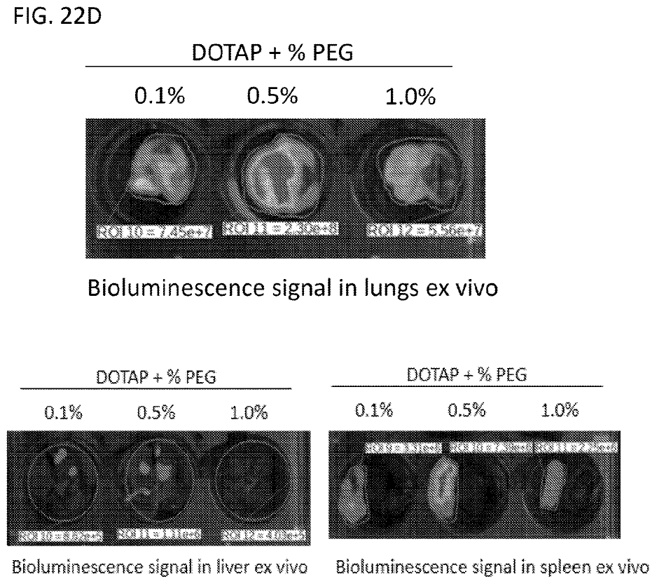

Methods: 2 lipid LNPs were formulated with firefly luciferase mRNA (Luc) per ratios in FIG. 22C. C57Bl/6 male mice were dosed at 1.25 mg/kg via intravenous injection (IV) and organs were imaged ex vivo 21 hr later. Shown in the graph is the mean radiance (photons/s/cm²/sr). FIGS. 22A-B, D.

Results: Bioluminescence signal was seen in the lung with all formulations, demonstrating: 1. There was toxicity at low PEG concentrations-one mouse died at 0.1% PE.G., suggesting the optimal amount may be between 0.2-2% PEG. 2. Signal peaked at 0.5% PEG.

### Example 6—Titrating Lipid:MRNA Ratio in 2 Lipid LNP

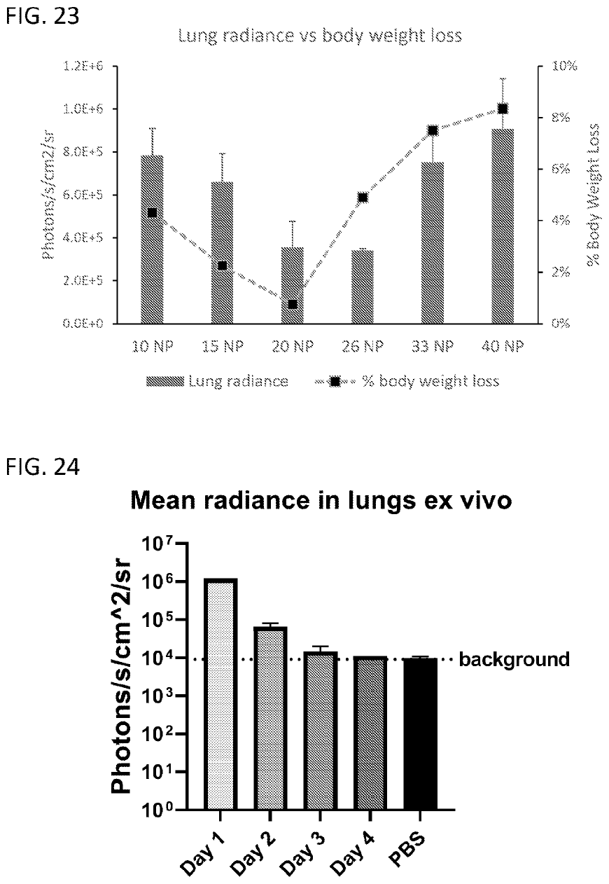

Methods: DOTAP and PEG were formulated with firefly luciferase mRNA (Luc). The lipid ratios were 99% DOTAP and 1% PEG. The lipid:mRNA ratios is presented as NP; the ratio of nitrogen to phosphate molecules. Each DOTAP molecule has a single nitrogen and each nucleoside on the mRNA has one phosphate. C57Bl/6 male mice were dosed at 1.5 mg/kg via intravenous injection (IV) and lungs were removed for ex vivo imaging 23 hr later. Shown in the FIG. 23 is the mean radiance (photons/s/cm²/sr).

Results: Bioluminescence signal was seen in the lung with all N/P ratios. An increase in lung signal corresponded to an increase in body weight loss-more signal, more toxicity. 20 Nitrogen:Phosphorus ratio (N/P) was a local minima for both signal and body weight loss

### Example 7—Time Course of Protein Expression after Delivery of Lung-Targeting mRNA-LNP Encoding Firefly Luciferase

Methods: LNPs was formulated with firefly luciferase mRNA (Luc) per ratios in the Table 3 of provisional. C57Bl/6 male mice were dosed at 1.5 mg/kg via intravenous injection (IV) and organs were imaged ex vivo 16 hr (“Day 1”), 33 hr (“Day 2”), 73 hr (“Day 3”), 91 hr (“Day 4”). Shown in the FIG. 24 is the mean radiance (photons/s/cm²/sr).

Results: Bioluminescence signal dropped over time as expected given the known half-life of firefly luciferase protein suggesting the mRNA half-life was not substantial, and that the mRNA-LNPs were not circulating and re-transfecting cells in the lung over-time.

### Example 8—Titrating SS-OP Lipid into the ‘DOTAP+PEG’ LNP

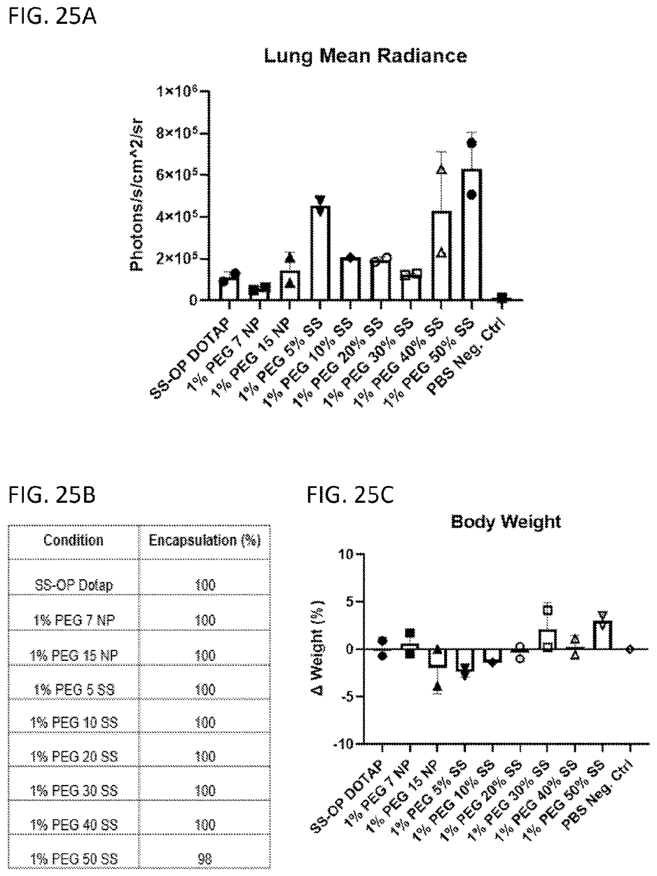

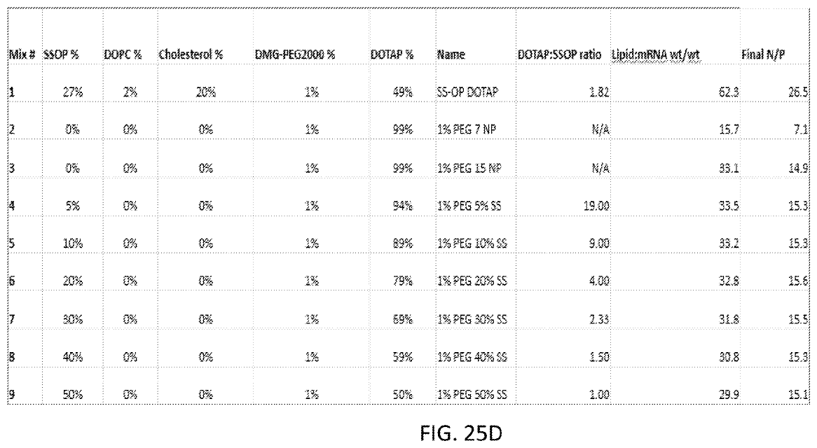

Methods: LNPs were formulated with firefly luciferase mRNA (Luc) per ratios in FIG. 25D. C57Bl/6 male mice were dosed at 1.5 mg/kg via intravenous injection (IV) and organs were imaged ex vivo 22 hr later. Shown in the FIG. 25A is the mean radiance (photons/s/cm²/sr).

### Results:

1. Encapsulation efficiency was 98% or greater as measured by Ribo-Green plate reader assay. FIG. 25B
2. Body weight was stable suggesting low or no toxicity. FIG. 25C
3. Lung radiance peaked at 50% SS-OP lipid
4. Local maxima at 5% SS-OP

### Example 9—Delivery to Lung and Liver Using 2 LNPs

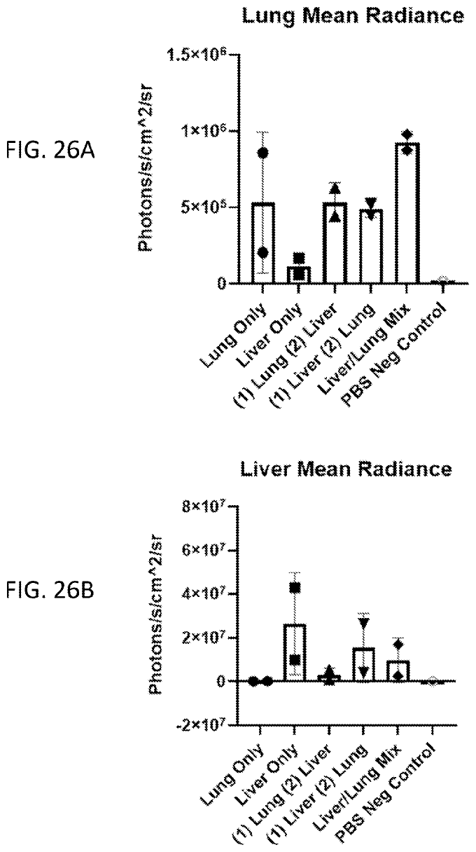

Methods: Liver-targeting LNP was formulated per lipid ratios in Table 4, and lung-targeting LNP was formulated per lipid ratios in Table 3. Both were formulated with firefly luciferase mRNA (Luc). CD1 male mice were dosed at 1.0 mg/kg via intravenous injection (IV) and organs were imaged ex vivo 19 hr later. Conditions:
Only lung-targeting LNP was dosed “Lung Only”
Only liver-targeting LNP was dosed “Liver Only”
Lung-targeting LNP was dosed first, then the liver-targeting LNP 1 hour later “(1) Lung (2) Liver
Liver-target LNP was dosed first, the lung-targeting LNP 1 hour later “(1) Liver (2) Lung”
Lung and liver-targeting LNPs were mixed and dosed twice, 1 hour apart “Liver/Lung Mix”
FIGS. 26A and 26B provide mean radiance (photons/s/cm²/sr).

TABLE 4

| Compound | Molar % |
|---|---|
| SS-OP | 54.2% |
| DOPC | 4.9% |
| Cholesterol | 39.4% |
| DMG-PEG2000 | 1.5% |

### Results:

1. Bioluminescence signal in lung was consistent in conditions where lung-targeting LNP was dosed. Highest was in the liver/lung mix. FIG. 26A
2. Liver BLI signal was variable, however it was very low in the condition where the lung LNP was dosed first. FIG. 26B.
3. Optimal sequence for both organs is lung first, then liver. Second optimal is to mix and deliver.

### Example 10—Titrating SS-OP Lipid into the ‘DOTAP+PEG’ LNP

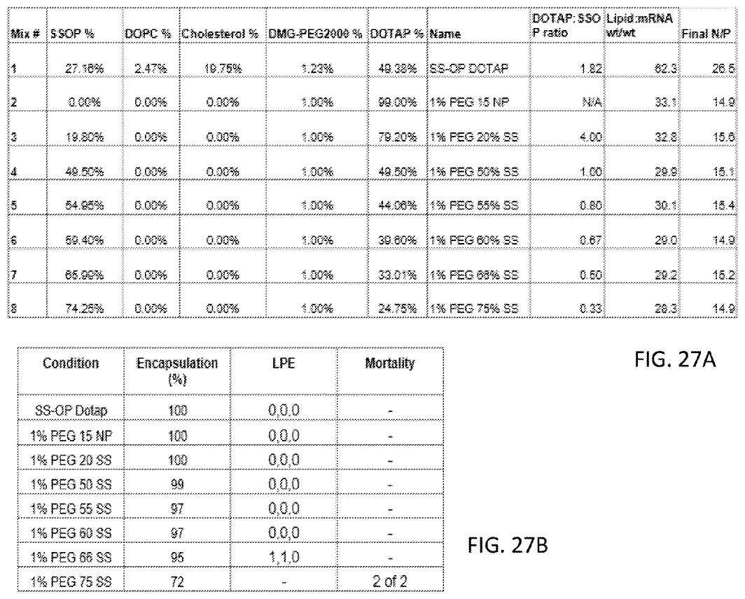

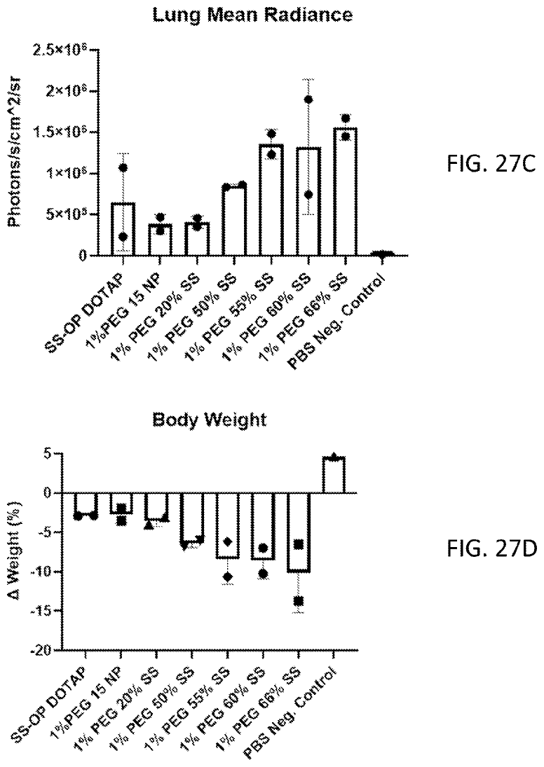

Methods: LNPs were formulated with firefly luciferase mRNA (Luc) per ratios in the table on FIG. 27A. C57Bl/6 male mice were dosed at 1.5 mg/kg via intravenous injection (IV) and organs were imaged ex vivo 23 hr later. Shown in the FIG. 27C is the mean radiance (photons/s/cm²/sr).

Body weights were measured and clinical signs assessed using a scoring system consisting of visual assessment of mouse appearance and behavior, specifically focusing on locomotion, posture, and eye (abbreviated “LPE”) (Nunamaker 2013, PMID: 24209966). FIG. 27D.

### Results:

1. Tolerability decreased when the proportion of SS-OP lipid was increased to 75%, at which both mice required euthanasia.
2. Combining data from Example 7, the studies suggest that 35-65% SS-OP provides relatively lung-specific transfection (as measured by bioluminescence signal) and relatively high tolerability (<10% body weight, no clinical signs (0 LPE), and no deaths) compared to other percentages of SS-OP. 55% SS-OP scored particularly well and may be considered optimal in these regards.

### Example 11—Stability Under Cryopreservation-Long Term

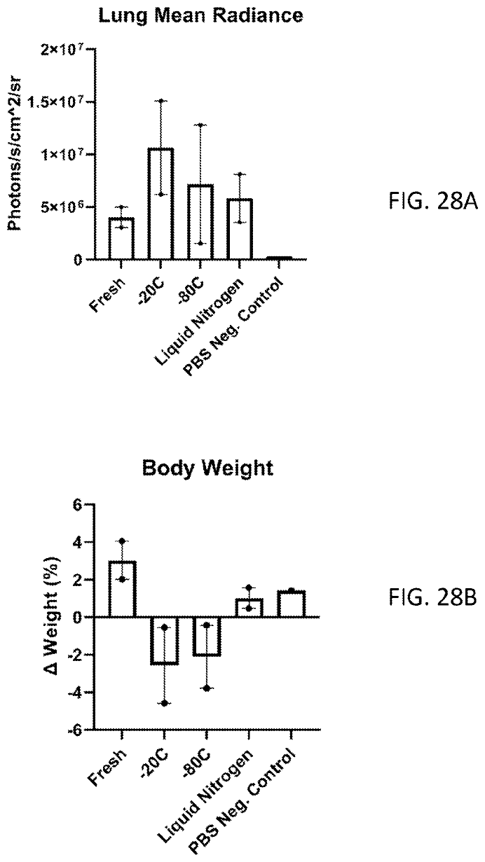

Methods: LNPs were formulated with firefly luciferase mRNA (Luc) per ratios in Table 3. They were either formulated and dosed on the same day ‘Fresh’ or cryopreserved at ~20 C, −80 C, or liquid nitrogen and thawed 70 days later. Sucrose was added to 7% prior to freezing as a cryoprotectant. CD1 male mice were dosed at 1.0 mg/kg via intravenous injection (IV) and organs were imaged ex vivo 23 hr later. Shown in the FIG. 28A is the mean radiance (photons/s/cm²/sr)+/−S.E.M.

Results: No clinical signs and minimal body weight loss was observed. FIG. 28B. Lung signal was present in all conditions at similar amounts suggesting cryopreservation was effective

### Example 12—Stability Under Cryopreservation-Short Term

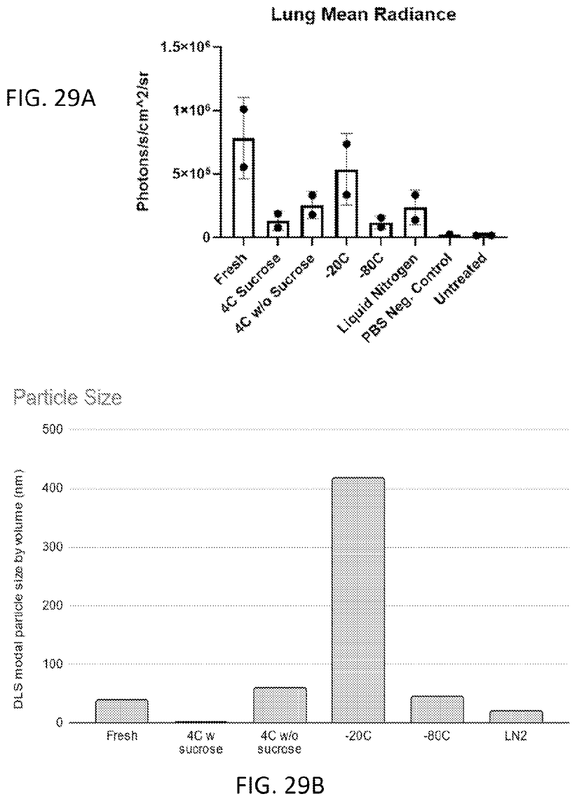

Methods: LNPs were formulated with firefly luciferase mRNA (Luc) per ratios in Table 3. They were either formulated and dosed on the same day ‘Fresh’ or cryopreserved at 4 C, −20 C, −80 C, or liquid nitrogen and thawed 3 days later. Sucrose was added to 15% prior to freezing as a cryoprotectant, except in the “4C w/o sucrose” condition. CD1 female mice were dosed at 0.4 mg/kg via intravenous injection (IV) and organs were imaged ex vivo 23 hr later. Shown in FIG. 29A is the mean radiance (photons/s/cm²/sr).

Results: Lung signal was lower than ‘fresh’ in the preserved conditions. By particle size measured with dynamic light scattering, 4 C with sucrose and −20 was out of range (see next slide). FIG. 29B.

### Example 13—Titrating N/P Ratios and Comparing 2, 3, and 5 Lipid Formulations

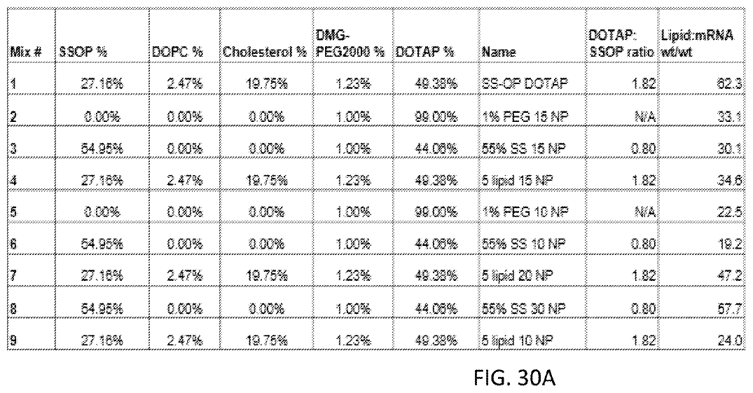

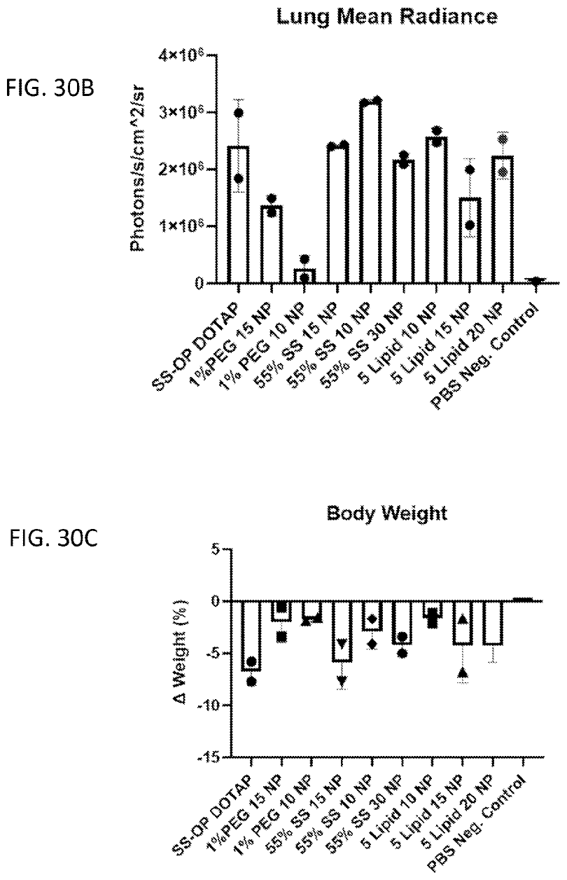

Methods: LNPs were formulated with firefly luciferase mRNA (Luc) per ratios in the FIG. 30A. C57Bl/6 male mice were dosed at 1.5 mg/kg via intravenous injection (IV) and organs were imaged ex vivo 23 hr later. Shown in FIG. 30B is the mean radiance (photons/s/cm²/sr).

### Results:

1. 3 and 5 lipid (SS and “5 Lipid”) formulations gave higher signal on average than the 2 lipid (1% PEG) formulation
2. In the 3 and 5 lipid formulations, 10 N/P was the optimal
3. 15 NP was better than 10 NP for 2 lipid
4. All formulations showed promise as lung-targeting LNP given high radiance and body weight loss below 10%, except for low activity of 2 lipid 10 N/P. FIG. 30C.

### Example 14—Titrating N/P Ratio (mRNA to Lipid Ratio) with 3 and 5 Lipid Formulations

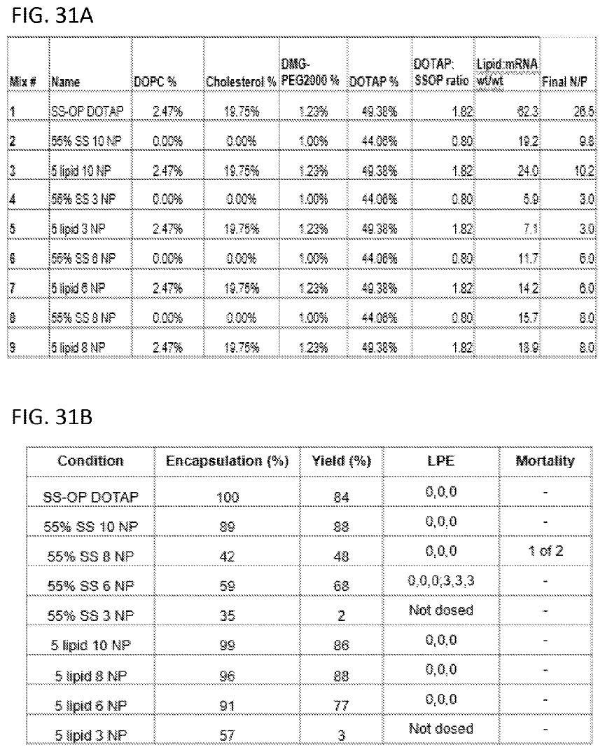

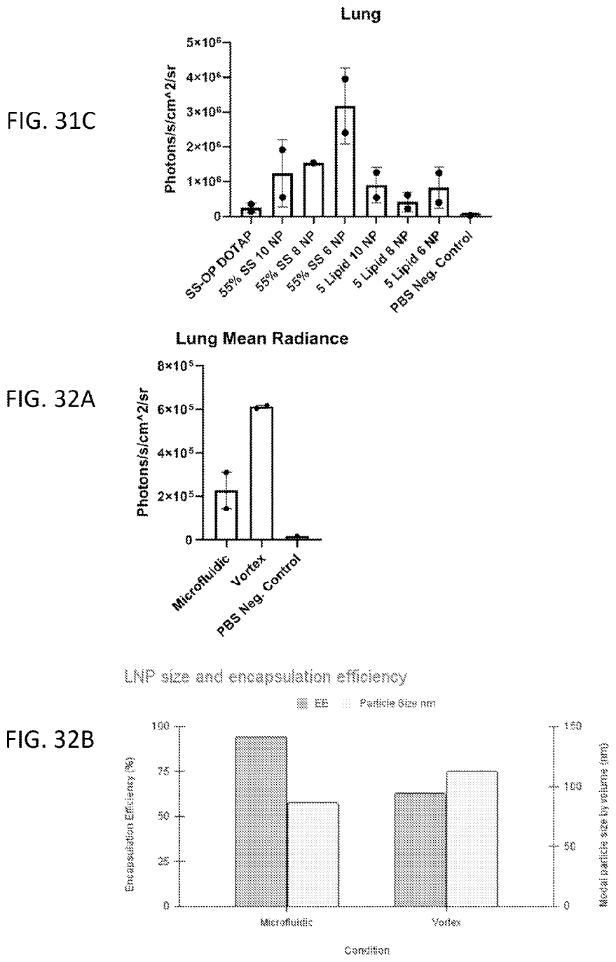

Methods: LNPs were formulated with firefly luciferase mRNA (Luc) per ratios in FIG. 31A. Bl/6 male mice were dosed at 1.5 mg/kg via intravenous injection (IV) and organs were imaged ex vivo 21 hr later. Shown in FIG. 31C is the mean radiance (photons/s/cm²/sr).

### Results:

1. There was significant toxicity in the 3 lipid formulations performed at 3 and 6 N/P
2. For 5 lipid lung-targeting LNPs, the encapsulation efficiency began dropping below 8 N/P. FIG. 31B
3. In this study 3 and 5 lipid with 10 N/P was superior overall given the above considerations

### Example 15—Testing Vortex Vs Microfluidic Mixing for mRNA-LNP Formulation

Methods: LNPs were formulated with firefly luciferase mRNA (Luc) per ratios in Table 3. Balb c/J male mice were dosed at 0.1 mg/kg via intravenous injection (IV) and organs were imaged ex vivo 21 hr later. Shown in FIG. 32A is the mean radiance (photons/s/cm²/sr).

### Results:

1. mRNA-LNPs mixed by vortexing produced LNP that yielded high BLI signal in the lung with slightly larger particle size (113 nm vs 86 nm). FIG. 32B.
2. mRNA-LNPs mixed by vortexing produced LNPs with encapsulation efficiency at 63%, far below the 94% of the microfluidic mixed counterpart. FIG. 32B.

### Example 16—Titrating NP Ratio for 5 Lipid Mix, Comparing Concentrations of Inputs

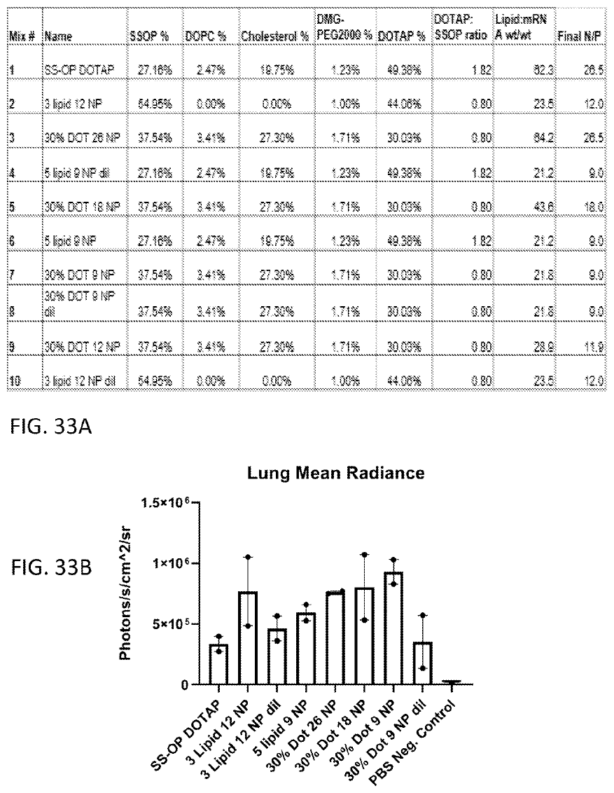

Methods: LNPs were formulated with firefly luciferase mRNA (Luc) per ratios in the table on FIG. 33A. C57Bl/6 male mice were dosed at 1.5 mg/kg via intravenous injection (IV) and organs were imaged ex vivo 21 hr later. Shown in FIG. 33B is the mean radiance (photons/s/cm²/sr).

For conditions labeled ‘dil’, the mRNA and lipid are diluted in buffer to 30% of original prior to mixing, but the ratios of the components is kept the same. The mRNA concentration for the input went from 0.147 mg/ml to 0.44 mg/ml.

### Results:

1. Decreasing DOTAP molar percentage from 49% to 30% led to a 30% improvement in signal in the 5 lipid formulation
2. Encapsulation efficiencies were above 90% for all formulations, and none of the mice exhibited abnormalities in locomotion, posture, or eyes (LPE score)

### Example 17—Titrating NP Ratio for 5 Lipid Mix; Comparing 5, 3, 2 Lipid LNP Formulations

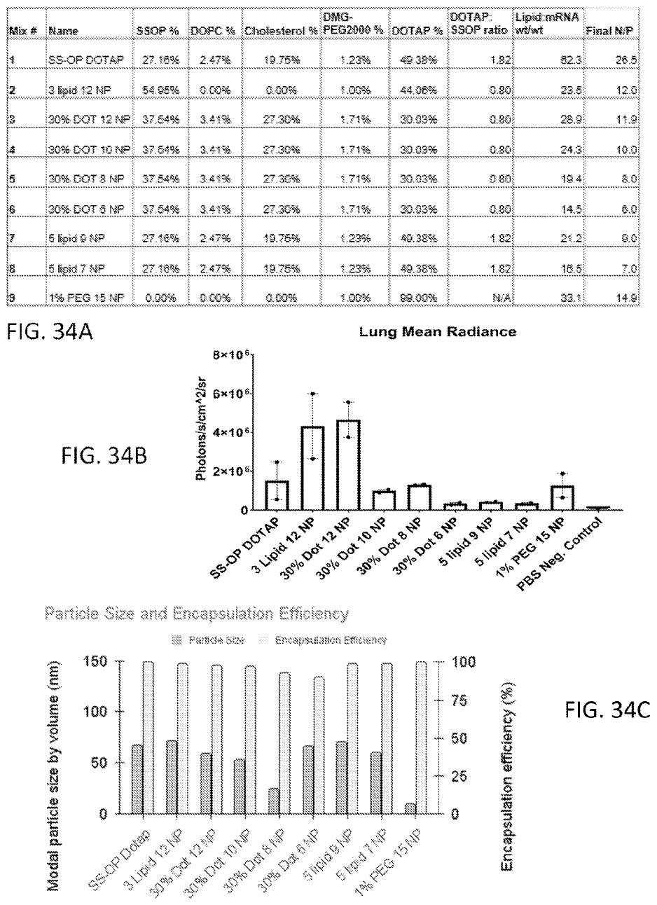

Methods: LNPs were formulated with firefly luciferase mRNA (Luc) per ratios in FIG. 34A. BALB/cJ male mice were dosed at 1.5 mg/kg via intravenous injection (IV) and organs were imaged ex vivo 20 hr later. Shown in FIG. 34B is the mean radiance (photons/s/cm²/sr). The formulations use 5 lipids with the exception of “3 Lipid 12 NP” and “1% PEG 15NP”.

Results: Encapsulation efficiency was ≥90% for all of the formulations. FIG. 34C. Top signal was observed with 12 NP for both 3 and 5 lipid lung-targeting LNPs.

### Example 18—Testing Different Total Flow Rates for 5-Lipid Formulation

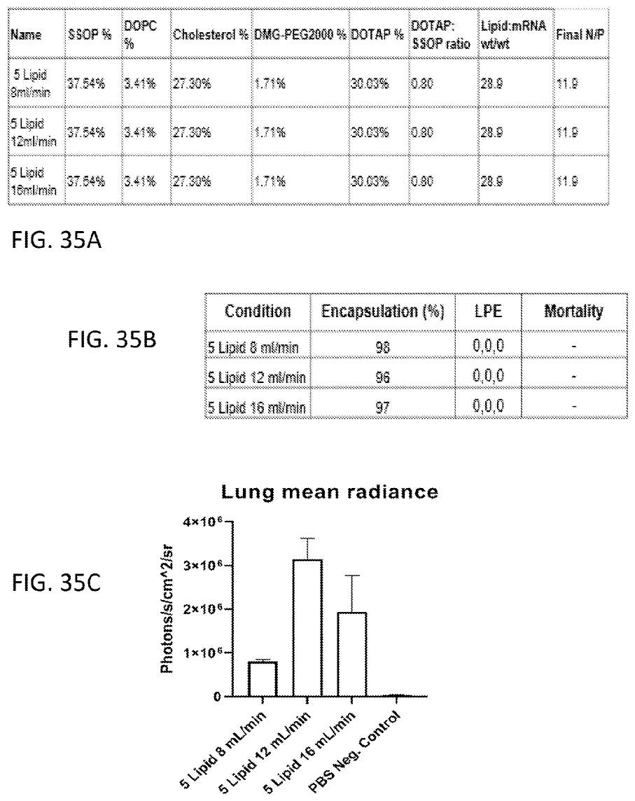

Methods: LNPs were formulated with firefly luciferase mRNA (Luc) per ratios in FIG. 35A. C57Bl/6 male mice were dosed at 2.0 mg/kg via intravenous injection (IV) and organs were imaged ex vivo 17 hr later. Shown in the FIG. 35C is the mean radiance (photons/s/cm²/sr). The formulations were the same, the variable changed was the flow rate through the Precision Nanosystems Nanoassemblr microfluidic mixing platform.

Results: There was high bioluminescence signal for all 3 flow rates, encapsulation efficiency >95%, and no clinical signs (LPE=0, no deaths). FIG. 35B. The preferred flow rate was 12 ml/min.

### Example 19—Testing Different Total Flow Rates for 3-Lipid Formulation (“PDS”)

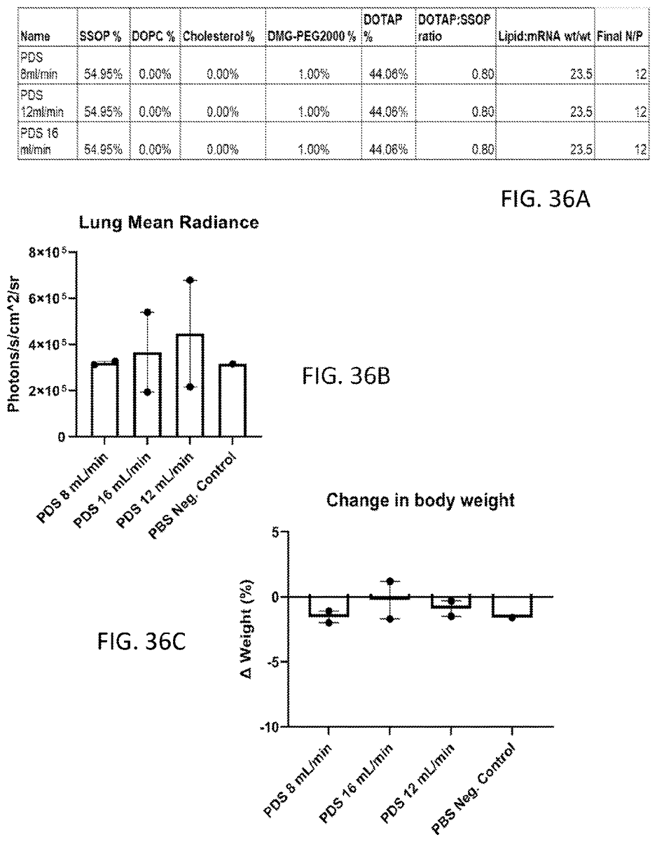

Methods: LNPs were formulated with firefly luciferase mRNA (Luc) per ratios in FIG. 36A. BALB/cJ male mice were dosed at 1.5 mg/kg via intravenous injection (IV) and organs were imaged ex vivo 26 hr later. Shown in FIG. 36B is the mean radiance (photons/s/cm²/sr). Total flow-rate was varied, but the ratio of the mRNA (aqueous) to lipid (organic) streams was maintained at 3:1. Thus the flow rate for the mRNA stream was total flow rate multiplied by 0.75 and the flow rate of the lipid stream was the total flow rate multiplied by 0.25.

### Results:

1. For all 3 flow-rates, bioluminescence signal was high, encapsulation efficiency was greater than 98%, and body weight loss was less than 5% (FIGS. 36B and 36C).

### Example 20-Substituting SS-OP in the 3-Lipid LNP with a Different Ionizable Lipid: DLin-MC3-DMA (“MC3”), Titrating MC3 vs DOTAP Ratios from 9 to 50% MC3

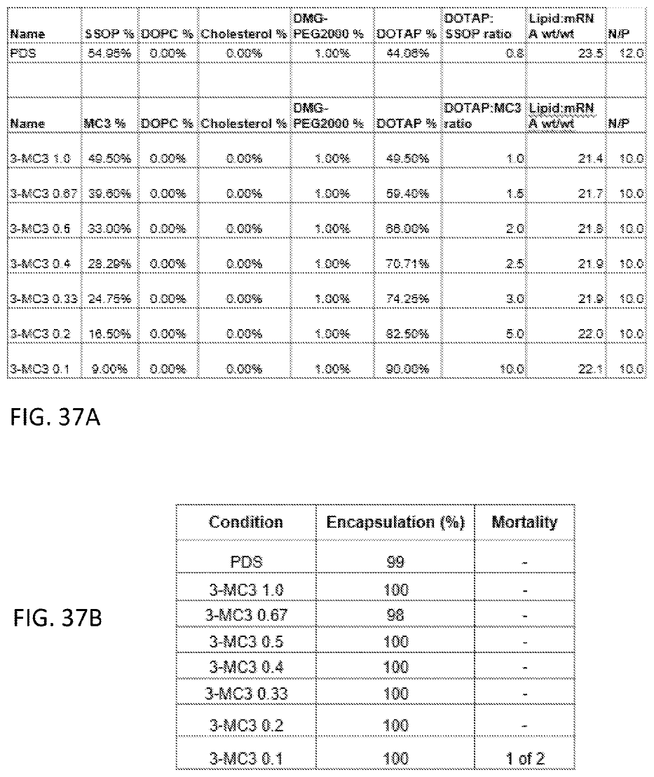

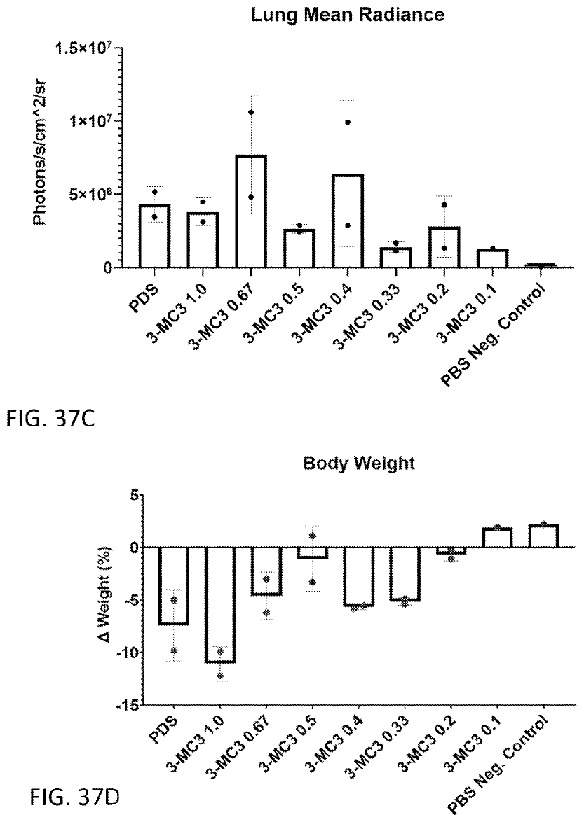

Methods: LNPs were formulated with firefly luciferase mRNA (Luc) per ratios in FIG. 37A. Encapsulation percentage for the LNPS is provided in FIG. 37B. BALB/cJ female mice were dosed at 2.0 mg/kg via intravenous injection (IV) and organs were imaged ex vivo 24 hr later. Shown in FIG. 37C is the mean radiance (photons/s/cm²/sr).

### Results:

1. Comparable bioluminescence signal in the lung was seen for all conditions, with a trend towards declining signal as MC3 concentration was reduced. FIG. 37C.
2. The lowest MC3 concentration (3-MC3 0.1) with 9% overall MC3 resulted in a mortality event. FIG. 37B.
3. Body weight loss trended upwards as MC3 concentration was increased, with the highest mean body weight loss at 50% MC3. FIG. 37D.

### Example 21—Swapping Out Ionizable Lipids SS-OP for DLin-MC3-DMA in 3 Lipid LNPs

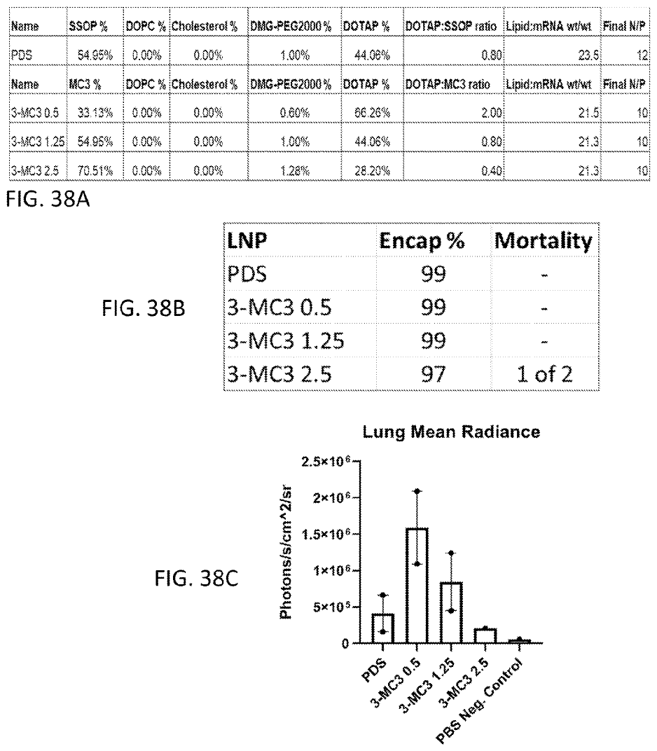

Methods: LNPs were formulated with firefly luciferase mRNA (Luc) per ratios in FIG. 38A. MC3 is short for DLin-MC3-DMA. CD1 female mice were dosed at 2.0 mg/kg via intravenous injection (IV) and organs were imaged ex vivo 26 hr later. Shown in FIG. 38C is the mean radiance (photons/s/cm²/sr).

### Results:

1. There was a mortality event in the LNP with 70.5% 3-MC, suggesting it was not well-tolerated.
2. Encapsulation efficiency was >95% in all conditions. FIG. 38B
3. Mean lung radiance was comparable, with the LNP formulation containing 33% molar percentage MC3 showing the highest signal on average.

### Example 22—Swapping Out Ionizable Lipids SS-OP for DLin-MC3-DMA in 5 Lipid LNPs

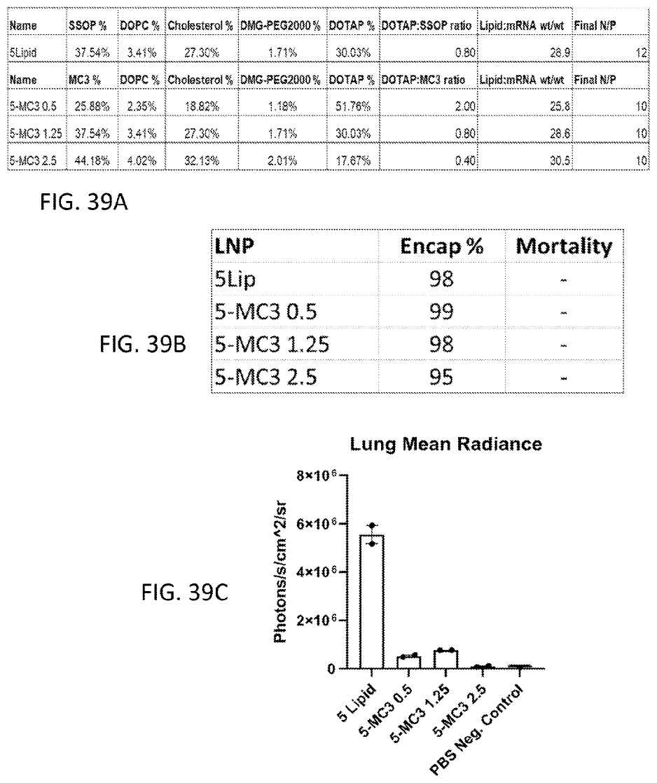

Methods: LNPs were formulated with firefly luciferase mRNA (Luc) per ratios in FIG. 39A. MC3 is short for DLin-MC3-DMA. CD1 female mice were dosed at 2.0 mg/kg via intravenous injection (IV) and organs were imaged ex vivo 26 hr later. Shown in FIG. 39C is the mean radiance (photons/s/cm²/sr).

### Results:

1. Encapsulation efficiency was ≥95% in all conditions. FIG. 39B.
2. Mean lung radiance was at least 7.2× greater in the 5-lipid condition with SS-OP than in any of the LNPs containing DLin-MC3-DMA. FIG. 39C.

### Example 23—Titrating Time that mRNA is Adsorbed to Formed LNP in 2-Lipid Formulation

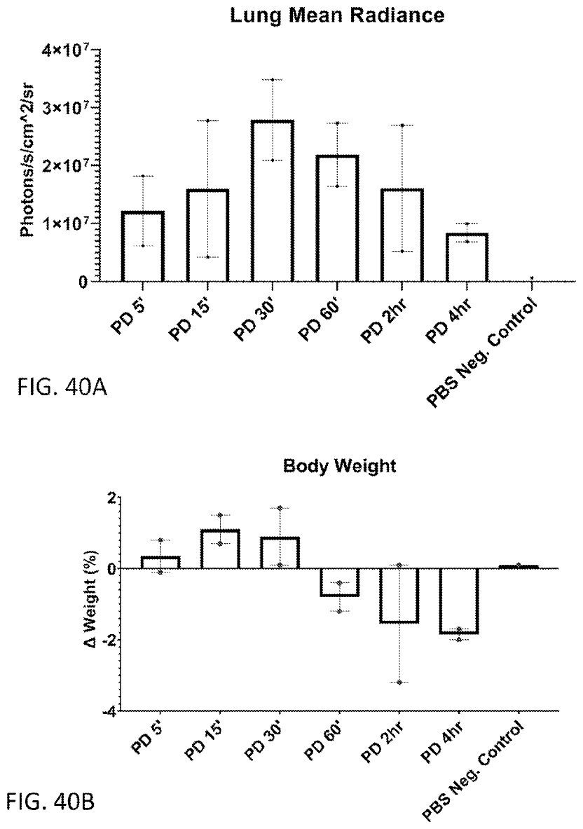

Methods: DOTAP and DMG-PEG2000 at a 100 to 1 molar ratio underwent microfluidic mixing with malic acid followed by buffer exchange to 0.1M sodium acetate and concentration. The lipid concentration was measured by HPLC with UV detector and then firefly luciferase mRNA (Luc) was added to the lipid to a final N/P ratio of 15. The mRNA was allowed to adsorb for the time noted in FIG. 40A before the addition of PBS. CD1 female mice were dosed at 2.0 mg/kg via intravenous injection (IV) and organs were imaged ex vivo 24 hr later. Shown in FIG. 40A is the mean radiance (photons/s/cm²/sr).

### Results:

1. Greatest lung signal was seen in the 30-minute incubation condition
2. Body weight loss was not more than 5% in any of the animals, and no deaths were observed suggesting tolerability. FIG. 40B

Encapsulation efficiency was greater than 98% for all conditions.

### Example 24—Titrating mRNA:Lipid Ratio for 2-Lipid LNP

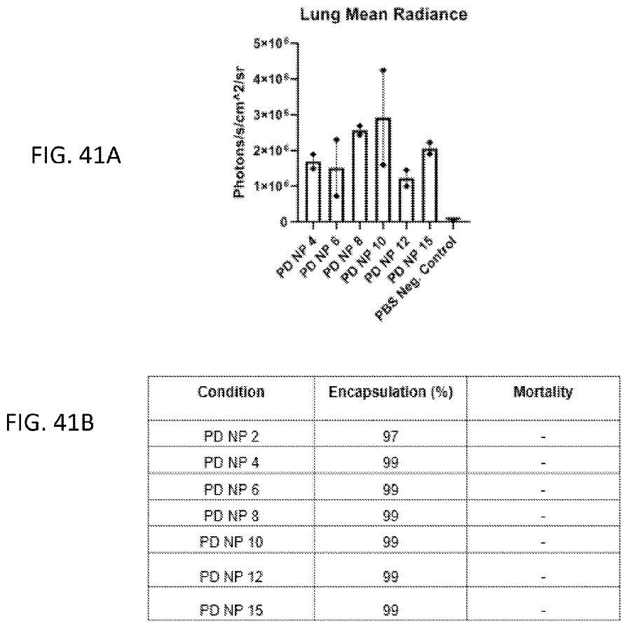

Methods: DOTAP and DMG-PEG2000 at a 100 to 1 molar ratio underwent microfluidic mixing with malic acid followed by buffer exchange to 0.1M sodium acetate and concentration. The lipid concentration was measured by HPLC and then firefly luciferase mRNA (Luc) was added to the lipid to an N/P ratio as denoted in FIG. 41A. Encapsulation percentage for each composition is provided in FIG. 41B. The mRNA was allowed to adsorb for 30 minutes before the addition of PBS. C57BL/6 female mice were dosed at 2.0 mg/kg via intravenous injection (IV) and organs were imaged ex vivo 27 hr later. Shown in FIG. 41A is the mean radiance (photons/s/cm²/sr).

### Results:

1. LNP with NP 2, not shown, did not yield enough mRNA to dose.
2. Lung bioluminescence signal was comparable between the different conditions

### Example 25—Titrating N/P Ratio for 3-Lipid LNP

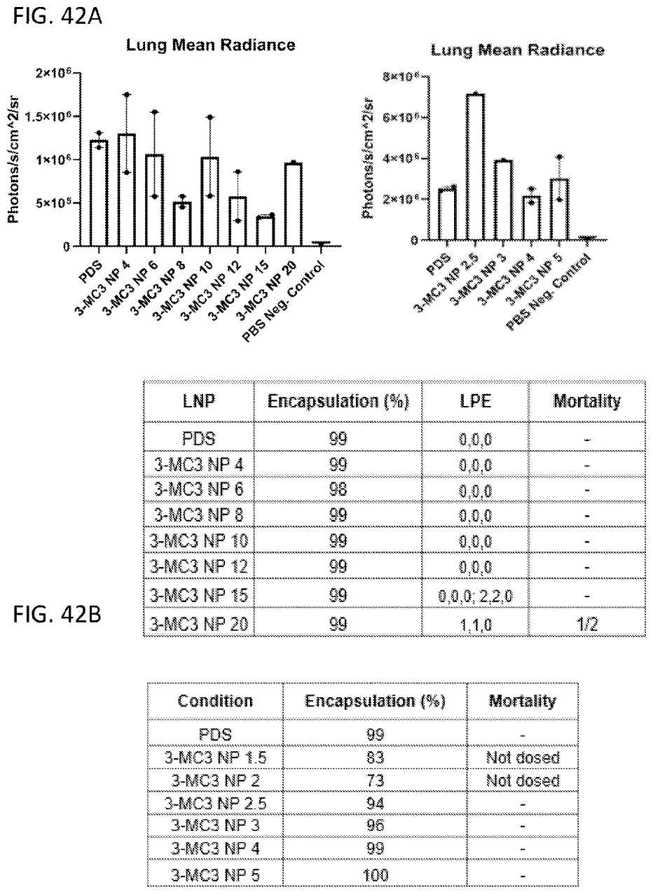

Methods: LNP “PDS” was formulated with firefly luciferase mRNA (Luc) per ratios in Table 6. Encapsulation percentage for each composition is provided in FIG. 42B. 3-MC3 LNPs were formulated with a molar percentage of 39.6% MC3, 59.4% DOTAP, and 1% DMG-PEG2000 at the N/P ratios listed in the graph C57BL/6 male mice were dosed at 2.0 mg/kg via intravenous injection (IV) and organs were imaged ex vivo 20 hr later. Shown in FIG. 42A is the mean radiance (photons/s/cm²/sr).

TABLE 6

| | | | | | | DOTAP: | Lipid: | |
|---|---|---|---|---|---|---|---|---|
| | SSOP | DOPC | Cholesterol | DMG-PEG2000 | DOTAP | SSOP | mRNA | |
| Name | % | % | % | % | % | ratio | wt/wt | Final N/P |
| PDS | 54.95% | 0.00% | 0.00% | 1.00% | 44.06% | 0.80 | 23.5 | 12.0 |

### Results:

1. There was a mortality event in the MC3 containing LNP with N/P ratio of 20, and clinical signs (slowed locomotion and hunched posture) in MC3 LNP with N/P ratio of 15, suggesting those were not well tolerated.
2. Encapsulation efficiency was >98% in all conditions
3. Lung mean radiance decreased with increasing N/P ratio
4. Very little mRNA-LNP was recovered in the NP 1.5 and NP 2 conditions, suggesting these mRNA: lipid ratios are not suitable for LNP formation in this context. NP 2.5 and NP 3 had low yields, so only a single animal was dosed.
5. DLin-MC3-DMA containing LNP with N/P ratios of 4 and 5 was the most promising in this study.

### Example 26—Titrating DMG-PEG 2000 in 2-Lipid LNP

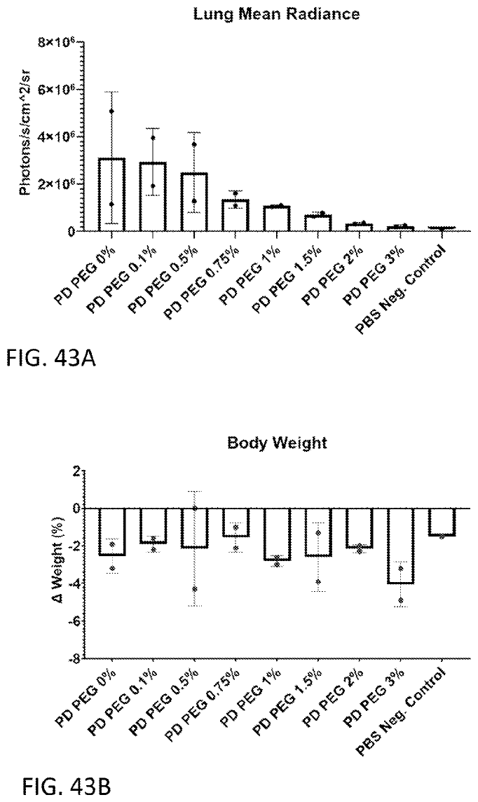

Methods: DOTAP and DMG-PEG2000 at a molar percentage as indicated in FIGS. 43A and 43B for the PEGylated lipid, underwent microfluidic mixing with malic acid followed by buffer exchange to 0.1M sodium acetate and concentration. The lipid concentration was measured by HPLC and then firefly luciferase mRNA (Luc) was added to a N/P ratio of 5.0. The mRNA was allowed to adsorb for 30 minutes before the addition of PBS. C57BL/6 female mice were dosed at 2.0 mg/kg via intravenous injection (IV) and organs were imaged ex vivo 20 hr later. Shown in FIG. 43A is the mean radiance (photons/s/cm²/sr).

### Results:

1. Single cationic lipid—DOTAP—is sufficient for tolerable lung-targeting.
2. The mean lung radiance decreased as DMG-PEG2000 was increased.

### Example 27—Microfluidic Vs Manual Mixing of Lipids in Ethanol with Malic Acid Buffer

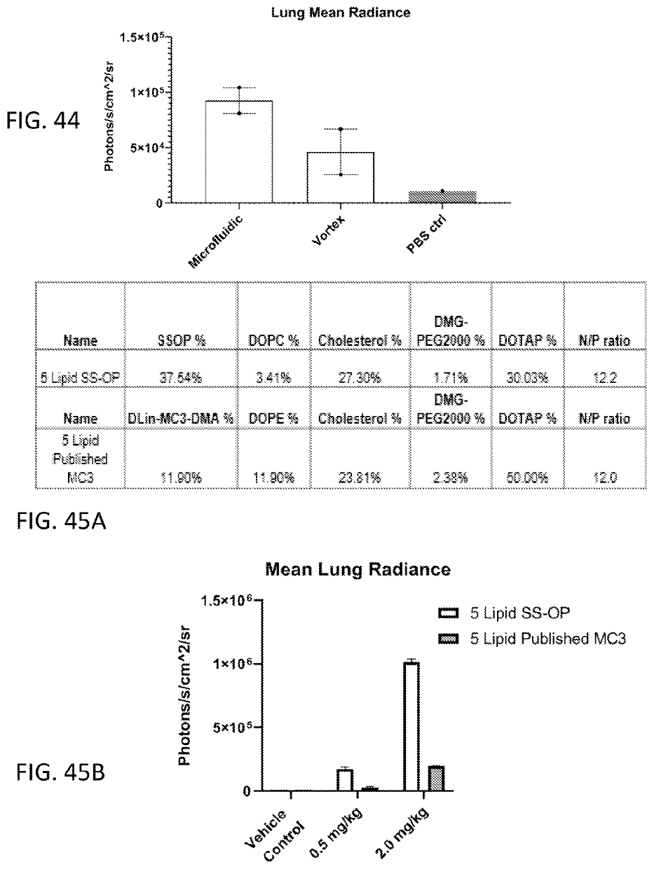

Methods: DOTAP and DMG-PEG2000 at a molar ratio of 100 to 1 underwent microfluidic or manual vortex mixing with malic acid followed by buffer exchange to 0.1M sodium acetate and concentration. The lipid concentration was measured by HPLC and then firefly luciferase mRNA (Luc) was added to a N/P ratio of 5.0. The mRNA was allowed to adsorb for 30 minutes before the addition of PBS. C57BL/6 female mice were dosed at 0.5 mg/kg via intravenous injection (IV) and organs were imaged ex vivo 20 hr later. Shown in FIG. 44 is the mean radiance (photons/s/cm²/sr).

### Results:

1. Microfluidic mixing resulted in 2× higher mean lung radiance
2. mRNA encapsulation efficiency was greater than 98% in both conditions

### Example 28—Comparing Published MC3 Formulation to 5-Lipid LNP with SS-OP

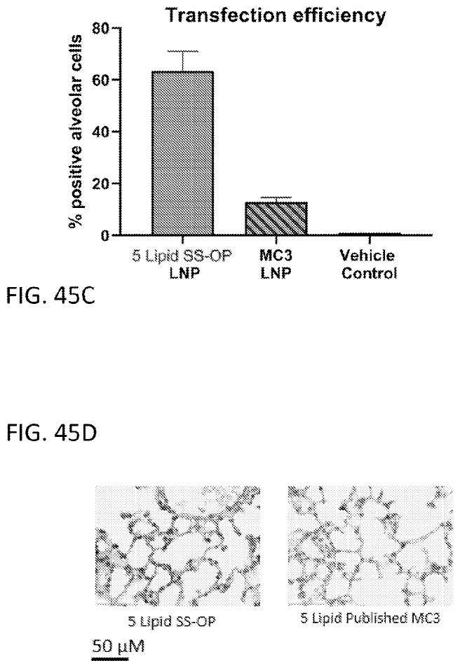

Methods 1: LNPs were formulated with firefly luciferase mRNA (Luc) per ratios in FIG. 45A. C57BL/6J female mice were dosed at the indicated mg/kg via intravenous injection (IV) and organs were imaged ex vivo 20 hr later. Shown in FIG. 45B is the mean radiance (photons/s/cm²/sr). The MC3 formulation was based on Dilliard et al 2021 PNAS (https://doi.org/10.1073/pnas.2109256118).

### Results 1:

1. The 5-lipid SS-OP containing LNP had 6.3× and 5.2 higher mean lung radiance compared to the published DLin-MC3-DMA containing lung-targeted LNP in the 0.5 and 2.0 mg/kg dose group respectively. FIG. 45B
2. Encapsulation was greater than >98% in both LNPs, and overall yields were comparable. Modal particle size by dynamic light scattering was between 35-55 nm.

Methods 2: LNPs were also formulated with Cre mRNA per ratios in FIG. 45A. The MC3 formulation was based on Dilliard et al 2021 PNAS (https://doi.org/10.1073/pnas.2109256118). Ai14 Tomato fl/fl female mice were dosed with Cre mRNA intravenously at 2.0 mg/kg. Lungs were harvested 3 days later and processed via formalin fixation and paraffin embedding. Positive cells were labeled with anti-tdTomato antibody. Shown in FIG. 45C are percent positive cells in the lung alveolar cells.

### Results 2:

1. The 5-lipid SS-OP containing LNP had 4.9× higher rate of transfection of lung alveolar cells.
2. Shown is a representative image of the lung alveolar region

### Example 29—Cell Transfection Rates in 3 Organs

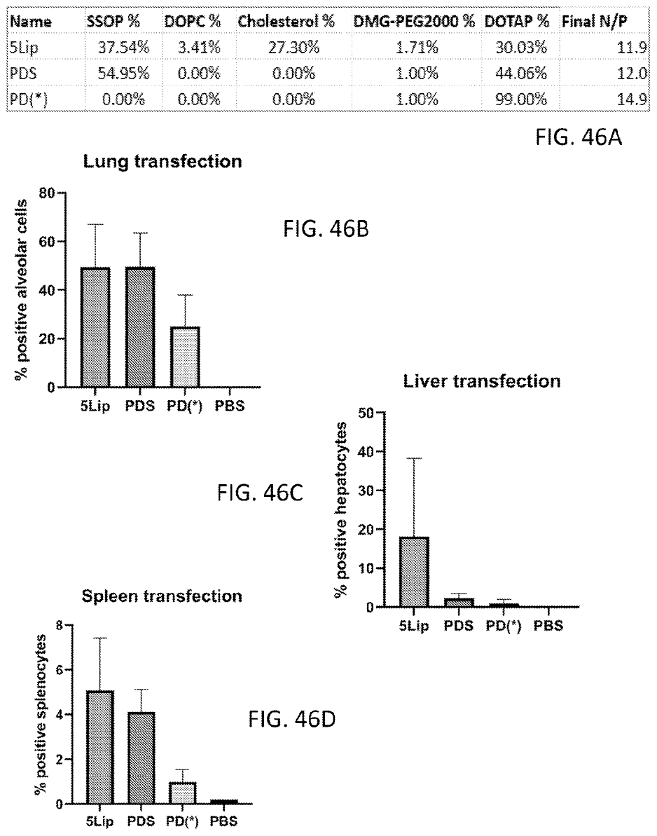

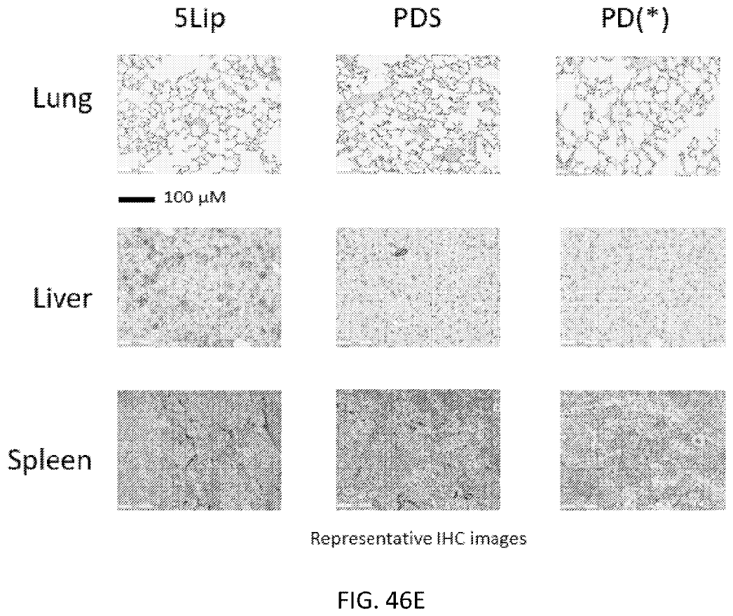

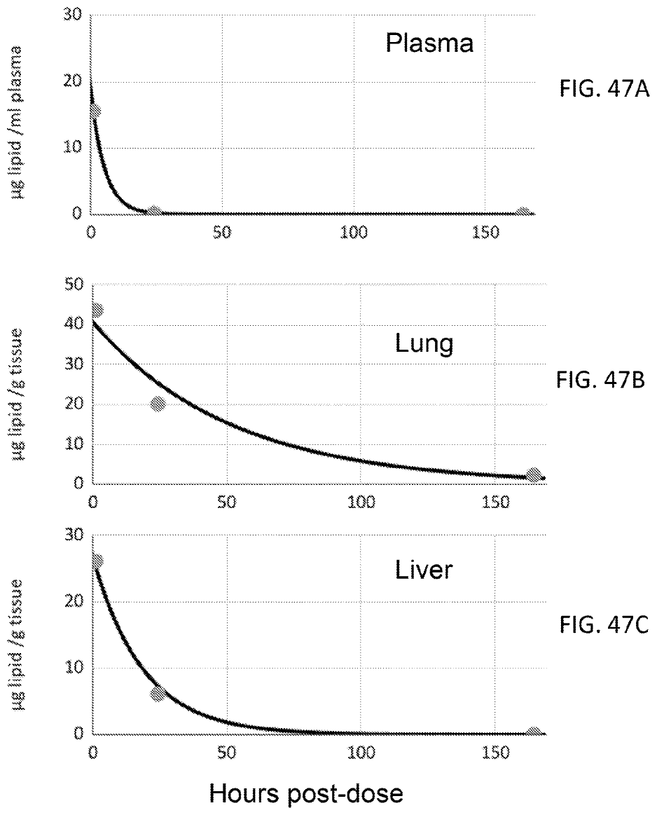

Methods: LNPs were formulated using lipid molar ratios and lipid:mRNA ratios as shown in FIG. 46A. The PD (*) formulation was prepared as described above (in Example 26). Ai14 Tomato fl/fl female mice were dosed with Cre mRNA intravenously at 1.5 mg/kg. Lungs were harvested 3 days later and processed via formalin fixation and paraffin embedding. Positive cells were labeled with anti-tdTomato antibody. Shown are percent positive cells in the lung alveolar space (FIG. 46B), liver hepatocytes (using nuclear size and circularity to exclude non-hepatocytes) (FIG. 46C), and splenocytes (FIG. 46D). Related IHC data are provided in FIG. 46E. Dark cells are Cre mRNA transfected tdTomato positive cells.

### Results:

1. The 5 lipid LNP (5Lip) and 3-lipid LNP (PDS) had comparable transfection in the lung and spleen, but the 5-lipid LNP had 8× higher transfection of hepatocytes in the liver.
2. Shown in FIG. 45D is a representative image of the lung alveolar region. Dark cells are transfected (tdTomato positive).

### Example 30—Pharmacokinetics with 2-Lipid LNP

Methods: The PD formulation was prepared as described above (in Example 26) with mRNA encoding human telomerase. As in FIG. 1, C57Bl/6 male mice were dosed at 1.0 mg/kg, and plasma and tissue were collected at the time points indicated. DOTAP was measured using LC-MS/MS.

| DMG-PEG2000% | DOTAP % | Lipid:mRNA wt/wt | N/P |
|---|---|---|---|
| 1.00% | 99.00% | 8.9 | 4.0 |

### Results:

| Tissue | Half-life (h) | Decay constant |
|---|---|---|
| Plasma | 3.57 | 0.194 |
| Lung | 35.7 | 0.0194 |
| Liver | 13.0 | 0.0534 |

### Example 31—Pharmacokinetics with 5-Lipid LNP

Methods: LNPs were formulated using lipid molar ratios in Table 1 with mRNA encoding human telomerase. CD1 male mice were dosed at 1.0 mg mRNA/kg of body weight, and plasma and tissue were collected at time points 0 (<5 min) and 24 hours. DOTAP was measured using LC-MS/MS. mRNA was measured by qRT-PCR. “Liver Rest” is the liver sample excluding the left lobe.

### Results:

| Molecule | Matrix | Half-life hr | Decay constant |
|---|---|---|---|
| DOTAP | Liver Left Lobe | 10.52 | 0.066 |
| DOTAP | Liver Rest | 13.79 | 0.050 |
| DOTAP | Lung Left Lobe | 7.37 | 0.094 |
| DOTAP | Lung Right Lobes | 9.51 | 0.073 |
| DOTAP | Spleen | 5.09 | 0.136 |
| DOTAP | Plasma | 3.63 | 0.191 |
| mRNA | Plasma | 3.26 | 0.212 |

### Example 32—Biodistribution with 5-Lipid LNP

Methods: LNPs were formulated using lipid molar ratios in Table 1 with mRNA encoding firefly Luciferase. New Zealand white rabbits were dosed intravenously (IV) at the level indicated in the graph for mg mRNA/kg of body weight. Organs were imaged ex vivo 6 hr later. Shown in the graph is the mean radiance (photons/s/cm²/sr).

Results are provided in FIGS. 49A and 49B.

### Example 33—Biodistribution with 3-Lipid LNP

Methods: LNPs were formulated using lipid molar ratios in Table 7 with mRNA encoding firefly luciferase. Cynomolgus macaques were dosed intravenously (IV) at the level indicated in the graph for mg mRNA/kg of body weight. Organs were imaged ex vivo 6 hr later after addition of luciferin. FIG. 50A is the mean radiance of each organ imaged (photons/s/cm²/sr). FIGS. 50B and 50C shows the relative radiance amongst the organs for the low and high dose: FIGS. 50D-50F show representative bioluminescent images of organs from a high dose animal. Shown in

TABLE 7

| Compound | Molar Percentage |
|---|---|
| SS-OP | 54.9% |
| DMG-PEG2000 | 1.0% |
| DOTAP | 44.1% |

Biodistribution results are provided for bioluminescence by organ depicted in terms of fold change of radiance are provided in FIG. 50G.

### Example 34—Telomerase Activity and Telomere Extension in Human Small Airway Epithelial Cells in Presence of SOC

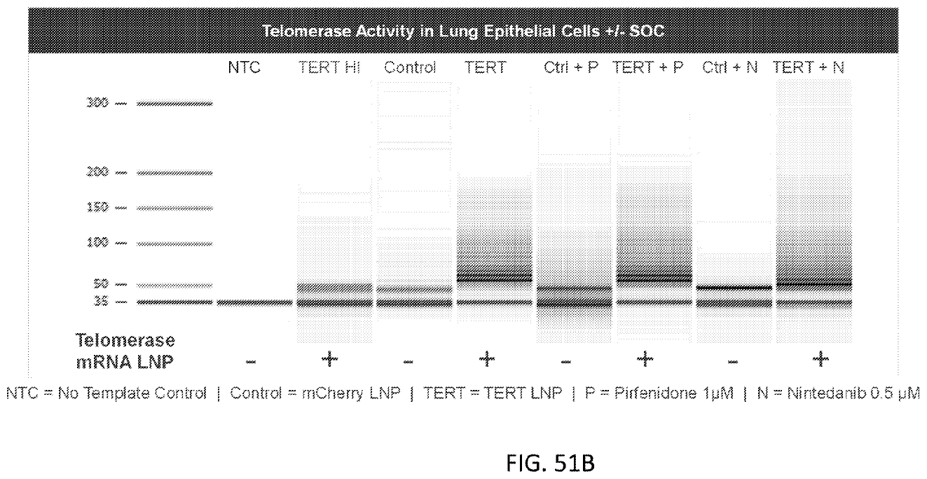

Methods: LNPs were formulated using lipid molar ratios in Table 3 with mRNA encoding human telomerase or mCherry as a control. LNP was added to human small airway epithelial cells (SAECs) obtained from Lonza in culture at 0.5 mg/ml. SAECs were grown in Lonza SAGM until 70% confluent One day prior, drugs representing standard-of-care (SOC) for patients with pulmonary fibrosis, pirfenidone (1 μM) and nintedanib (0.5 μM), were added to the culture, and replaced at every media change. For measurement of telomerase activity, cells were harvested and lysed in CHAPS buffer for the TRAP assay 24 hours post treatment with LNP. The lysate was exposed to an artificial telomerase single-stranded DNA template, and PCR amplification was used to detect telomerase activity. FIG. 51B.

For telomere length measurement, the cells were harvested and fixed five days post addition of telomerase mRNA LNPs. FIG. 51A. The Q-FISH protocol was performed as follows: cells were spun down on glass slides and were permeabilized and had the telomeres labeled with a fluorescent probe. FIG. 51B. Microscopy and quantitative image analysis were performed to determine lengths of individual telomeres. All of the hTERT LNP samples (+/−SOC) were pooled in the analysis to test for differences in telomere length.

### Results:

1. Telomerase activity was detected in human small airway epithelial cells after addition of hTERT LNPs
2. Telomerase activity was detected at similar levels in the cells when standard of care drugs were added
3. Median and 20^{th} percentile telomere length were significantly increased by 492 and 435 base pairs respectively after addition of a single dose of hTERT LNPs.

### Example 35—Telomerase Activity and Telomere Extension in Human Lung Fibroblasts in Presence of SOC

Methods: LNPs were formulated using lipid molar ratios in Table 3 with mRNA encoding human telomerase or mCherry as a control. LNP was added to fetal human lung fibroblast MRC-5 cells (passage 5) were grown in DMEM+10% FBS until 70-80% confluency. 24 hrs prior to the LNP treatment MRC-5 cells were pre-treated with the standard-of-care drugs pirfenidone (1 μM) and nintedanib (1 uM), following by treatment with 500 ng/ml hTERT LNPs or 500 ng/ml of mCherry LNPs (control). Standard-of-care drugs were added fresh with every media change.

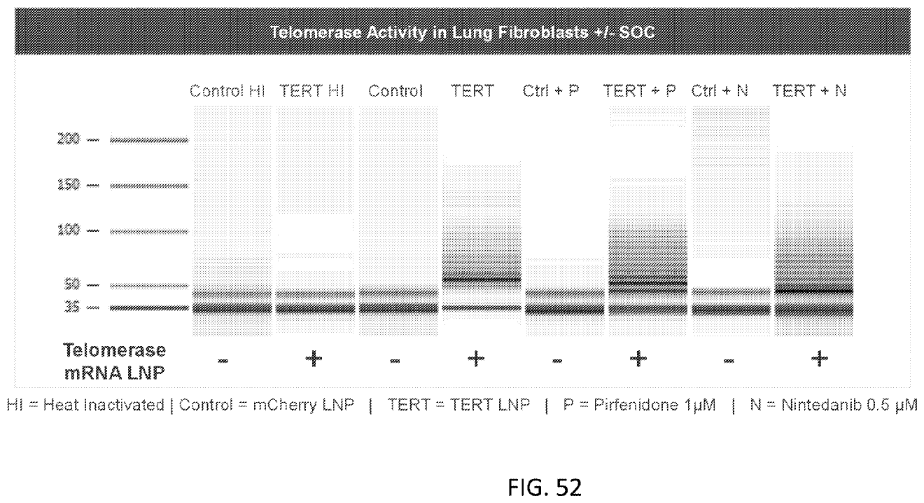

For measurement of telomerase activity, cells were harvested and lysed in CHAPS buffer for the TRAP assay 24 hours post treatment with LNP. The lysate was exposed to an artificial telomerase single-stranded DNA template, and PCR amplification was used to detect telomerase activity. FIG. 52.

### Results:

1. Telomerase activity was detected in human lung fibroblasts after addition of hTERT LNPs.
2. Telomerase activity was detected at similar levels in the cells when standard of care drugs were added.

### Example 36—Lyophilization for 3 and 5 Lipid LNPs

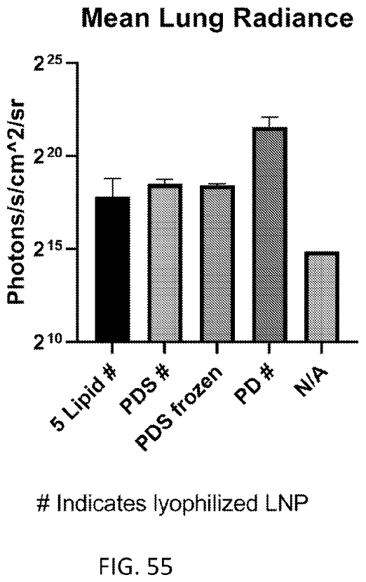

Methods: LNPs are formulated using lipid molar ratios described herein with mRNA encoding firefly luciferase. The LNP ratios used are described in Table 8. LNPs were mixed with 15% sucrose and frozen at −80° C. Next, they were placed into a pre-frozen shelf of the L-200 Pro Lyovapor freeze dryer. The vacuum was set to 1 mbar, the condenser was set to −55° C., and the total run-time was 22 hours. The LNPs were stored at 4° C. for 2 days prior to resuspension with water. As a control “PDS” LNP was frozen with 15% sucrose and thawed without lyophilization. 2^{nd} generation Tert −/− female mice on a C57Bl/6 background are dosed at 0.3 mg mRNA/kg of body weight via intravenous injection (IV) and organs are imaged ex vivo 18 hr later. FIG. 55.

Results: In vivo activity for 5-Lipid and PDS was comparable to each other and to the frozen PDS LNP which was not lyophilized (freeze-dried). The freeze-dried and reconstituted PD LNP exhibited at least 8× higher activity compared to the other LNPs.

### Example 37—Summary of Exemplary LNP Formulations of the Present Disclosure

Table 8 provide the molar percentage composition, N/P ratio, molar ratio of cationic lipid:ionizable lipid, and lipid:mRNA ratio (wt/wt), of exemplary LNPs of the present disclosure named, respectively, “SSOP-DOTAP”, “5 lipid”, “PDS”, “3-MC3”, “PD”

TABLE 8

| Compound or ratio | | | | | |
|---|---|---|---|---|---|
| (below); LNP name | SSOP- | 5 | | 3- | |
| (right) | DOTAP | Lipid | PDS | MC3 | PD |
| SS-OP | 27.2% | 37.5% | 54.9% | 0.0% | 0.0% |
| DLin-MC3-DMA | 0.0% | 0.0% | 0.0% | 39.6% | 0.0% |
| DMG-PEG2000 | 1.2% | 1.7% | 1.0% | 1.0% | 0.0% |
| DOTAP | 49.4% | 30.0% | 44.1% | 59.4% | 99.0% |
| DOPC | 2.5% | 3.4% | 0.0% | 0.0% | 0.0% |
| Cholesterol | 19.8% | 27.3% | 0.0% | 0.0% | 0.0% |
| N/P Ratio | 29.1 | 12.1 | 12.0 | 4.5 | 4.0 |
| Cationic lipid:ionizable | 1.8 | 0.8 | 0.8 | 1.5 | N/A |
| lipid (C/I) | | | | | |
| (molar ratio) | | | | | |

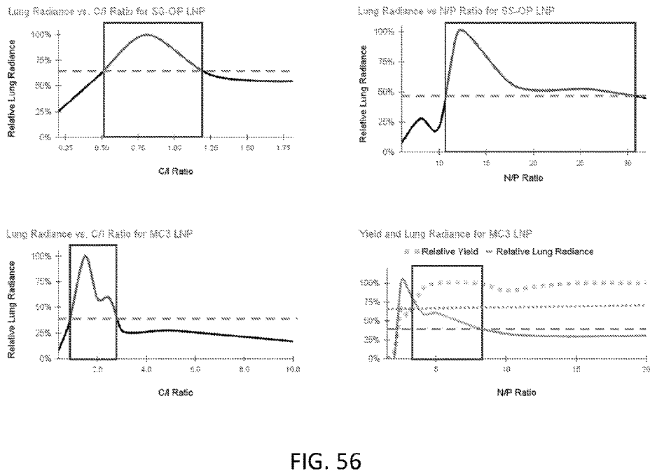

Optimal cationic lipid to ionizable lipid ratios (“C/I Ratio”) and N/P ratios identified by the present inventors for LNPs containing either the SS-OP family or DLin-MC3-DMA family of ionizable lipids are modeled in FIG. 56.

### Example 38—Adding mRNA after LNP is Formed

LNPs were formulated with firefly luciferase mRNA (Luc) per ratios in Table 8. For LNPs with an asterisk (*), the microfluidic mixing was performed with buffer only, no mRNA. This was followed by buffer exchange to 0.1M sodium acetate and concentration. The firefly luciferase mRNA (Luc) was added to the lipid to an N/P ratio as denoted in the graph. The mRNA was allowed to adsorb for 2 hours before the addition of PBS, 0.2 μM filtration, and dosing. mRNA LNPs were delivered intravenously to C57BL/6 male mice and organs were imaged ex vivo 17 hr post.

### Results:

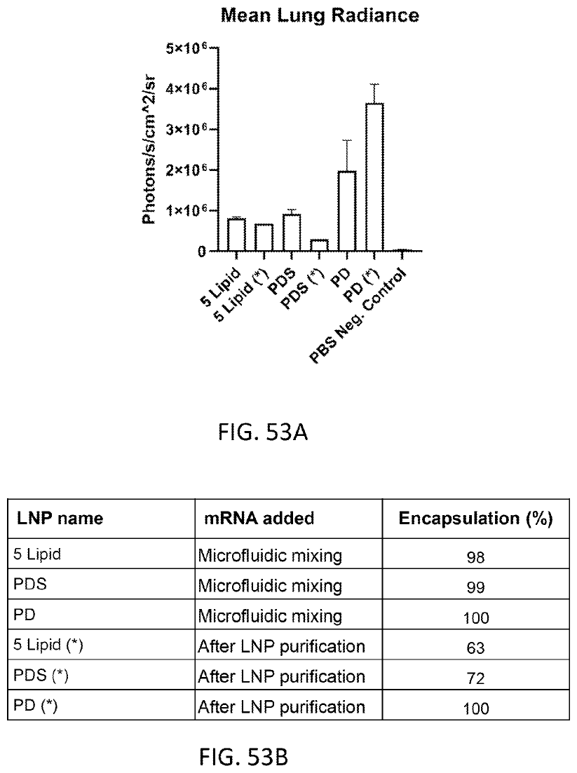

1. Adding the mRNA at the end resulted in the following change in mean lung radiance (FIG. 53A):
   85% increase for PD LNP
   68% decrease for PDS LNP
   16% decrease for 5 Lipid
2. Encapsulation efficiency was >95% only in the PD (*) condition, not the PDS (*) and 5 Lipid (*) where the mRNA was added at the conclusion of the purification process. FIG. 53B.

### Example 39—Adding mRNA after LNP is Formed

LNPs were formulated with firefly luciferase mRNA (Luc) per ratios in Table 8. For PD (*), the microfluidic mixing was performed with buffer only, no mRNA. This was followed by buffer exchange to 0.1M sodium acetate and concentration. The firefly luciferase mRNA (Luc) was added to the lipid to an N/P ratio as denoted in the graph. The mRNA was allowed to adsorb for 2 hours before the addition of PBS, 0.2 μM filtration, and dosing. mRNA LNPs were delivered intravenously to BALB/cJ male mice at 1.5 mg/kg and organs were imaged ex vivo 26 hr post.

### Results:

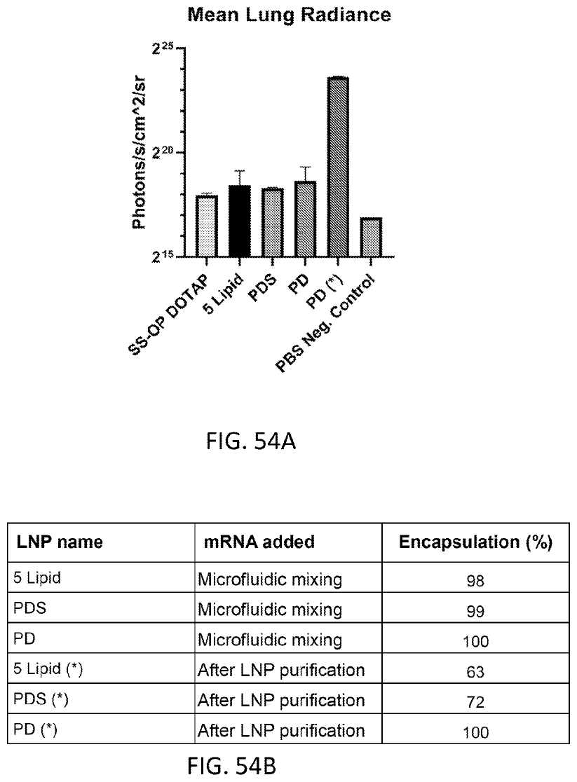

1. Adding the mRNA at the end for the PD formulation resulted in a 31.8× increase in bioluminescence signal. FIG. 54A.
2. Encapsulation efficiency was >95% for all formulations. FIG. 54B.

A listing of exemplary nucleic acid sequences used in the presently described examples is provided below. Additional sequences, such as those set forth in PCT/US22/22642, filed Mar. 30, 2022, U.S. application Ser. No. 17/709,108, filed Mar. 30, 2022, and U.S. Provisional Application No. 63/169,118, filed Mar. 31, 2021 are incorporated herein by reference.

SEQ ID NO: 1
atgcccagag cccccagatg cagagccgtg agaagcctgc tgagaagcca ctacagagag 60
gtgctgcccc tggccacctt cgtgagaaga ctgggccccc agggctggag actggtgcag 120
agaggcgacc ccgccgcctt cagagccctg gtggcccagt gcctggtgtg cgtgccctgg 180
gacgccagac cccctcccgc cgcccccagc ttcagacagg tgagctgcct gaaggagctg 240
gtggccagag tgctgcagag actgtgcgag agaggcgcca agaacgtgct ggccttcggc 300
ttcgccctgc tggacggcgc cagaggcggc ccccccgagg ccttcaccac cagcgtgaga 360
agctacctgc ccaacaccgt gaccgacgcc ctgagaggca gcggcgcctg gggcctgctg 420
ctgagaagag tgggcgacga cgtgctggtg cacctgctgg ccagatgcgc cctgttcgtg 480
ctgg ggccc ccagctgcgc ctaccaggtg tgcggccctc ccctgtacca gctgggcgcc 540
gccacccagg ccagaccccc tccccacgcc agcggcccca gaagaagact gggctgcgag 600
agagcctgga accacagcgt gagagaggcc ggcgtgcccc tgggcctgcc cgcccccggc 660
gccagaagaa gaggcggcag cgccagcaga agcctgcccc tgcccaagag acccagaaga 720
ggcgccgccc ccgagcccga gagaaccccc gtgggccagg gcagctgggc ccaccccggc 780
agaaccagag gccccagcga cagaggcttc tgcgtggtga gccccgccag acccgccgag 840
gaggccacca gcctggaggg cgccctgagc ggcaccagac acagccaccc cagcgtgggc 900
agacagcacc acgccggccc tcccagcacc agcagacctc ccagaccctg ggacaccccc 960
tgccctcccg tgtacgccga gaccaagcac ttcctgtaca gcagcggcga caaggagcag 1020
ctgagaccca gcttcctgct gagcagcctg agacccagcc tgaccggcgc cagaagactg 1080
gtggagacca tcttcctggg cagcagaccc tggatgcccg gcacccccag aagactgccc 1140
agactgcccc agagatactg gcagatgaga cccctgttcc tggagctgct gggcaaccac 1200
gcccagtgcc cctacggcgt gctgctgaag acccactgcc ccctgagagc cgccgtgacc 1260
cccgccgccg gcgtgtgcgc cagagagaag ccccagggca gcgtggccgc ccccgaggag 1320
gaggacaccg accccagaag actggtgcag ctgctgagac agcacagcag cccctggcag 1380
gtgtacggct tcgtgagagc ctgcctgaga agactggtgc ctcccggcct gtggggcagc 1440
agacacaacg agagaagatt cctgagaaac accaagaagt tcatcagcct gggcaagcac 1500
gccaagctga gcctgcagga gctgacctgg aagatgagcg tgagagactg cgcctggctg 1560
agaagaagcc ccggcgtggg ctgcgtgccc gccgccgagc acagactgag agaggagatc 1620
ctggccaagt tcctgcactg gctgatgagc gtgtacgtgg tggagctgct gagaagcttc 1680
ttctacgtga ccgagaccac cttccagaag aacagactgt tcttctacag aaagagcgtg 1740
tggagcaagc tgcagagcat cggcatcaga cagcacctga agagagtgca gctgagagag 1800
ctgagcgagg ccgaggtgag acagcacaga gaggccagac ccgccctgct gaccagcaga 1860
ctgagattca tccccaagcc cgacggcctg agacccatcg tgaacatgga ctacgtggtg 1920
ggcgccagaa ccttcagaag agagaagaga gccgagagac tgaccagcag agtgaaggcc 1980
ctgttcagcg tgctgaacta cgagagagcc agaagacccg gcctgctggg cgccagcgtg 2040
ctgggcctgg acgacatcca cagagcctgg agaaccttcg tgctgagagt gagagcccag 2100
gatccccctc ccgagctgta cttcgtgaag gtggacgtga ccggcgccta cgacaccatc 2160
ccccaggaca gactgaccga ggtgatcgcc agcatcatca agccccagaa cacctactgc 2220
gtgagaagat acgccgtggt gcagaaggcc gcccacggcc acgtgagaaa ggccttcaag 2280
agccacgtga gcaccctgac cgacctgcag ccctacatga gacagttcgt ggcccacctg 2340
caggagacca gccccctgag agacgccgtg gtgatcgagc agagcagcag cctgaacgag 2400
gccagcagcg gcctgttcga cgtgttcctg agattcatgt gccaccacgc cgtgagaatc 2460
agaggcaaga gctacgtgca gtgccagggc atcccccagg gcagcatcct gagcaccctg 2520
ctgtgcagcc tgtgctacgg cgacatggag aacaagctgt tcgccggcat cagaagagac 2580
ggcctgctgc tgagactggt ggacgacttc ctgctggtga caccccacct gacccacgcc 2640
aagaccttcc tgagaaccct ggtgagaggc gtgcccgagt acggctgcgt ggtgaacctg 2700
agaaagaccg tggtgaactt ccccgtggag gacgaggccc tgggcggcac cgccttcgtg 2760
cagatgcccg cccacggcct gttcccctgg tgcggcctgc tgctggacac cagaaccctg 2820
gaggtgcaga gcgactacag cagctacgcc agaaccagca tcagagccag cctgaccttc 2880
aacagaggct tcaaggccgg cagaaacatg agaagaaagc tgttcggcgt gctgagactg 2940
aagtgccaca gcctgttcct ggacctgcag gtgaacagcc tgcagaccgt gtgcaccaac 3000
atctacaaga tcctgctgct gcaggcctac agattccacg cctgcgtgct gcagctgccc 3060
ttccaccagc aggtgtggaa gaaccccacc ttcttcctga gagtgatcag cgacaccgcc 3120
agcctgtgct acagcatcct gaaggccaag aacgccggca tgagcctggg cgccaagggc 3180
gccgccggcc ccctgcccag cgaggccgtg cagtggctgt gccaccaggc cttcctgctg 3240
aagctgacca gacacagagt gacctacgtg cccctgctgg gcagcctgag aaccgcccag 3300
acccagctga gcagaaagct gcccggcacc accctgaccg ccctggaggc cgccgccaac 3360
cccgccctgc ccagcgactt caagaccatc ctggactga 3399
SEQ ID NO: 2
aggaaataag agagaaaaga agagtaagaa gaaatataag agccaccatg acccgcgctc 60
ctcgctgccc cgcggtgcgc tctctgctgc gcagccgata ccgggaggtg tggccgctgg 120
caacctttgt gcggcgcctg gggcccgagg gcaggcggct tgtgcaaccc ggggacccga 180
agatctaccg cactttggtt gcccaatgcc tagtgtgcat gcactggggc tcacagcctc 240
cacctgccga cctttccttc caccaggtgt catccctgaa agagctggtg gccagggttg 300
tgcagagact ctgcgagcgc aacgagagaa acgtgctggc ttttggcttt gagctgctta 360
acgaggccag aggcgggcct cccatggcct tcactagtag cgtgcgtagc tacttgccca 420
acactgttat tgagaccctg cgtgtcagtg gtgcatggat gctactgttg agccgagtgg 480
gcgacgacct gctggtctac ctgctggcac actgtgctct ttatcttctg gtgcccccca 540
gctgtgccta ccaggtgtgt gggtctcccc tgtaccaaat ttgtgccacc acggatatct 600
ggccctctgt gtccgctagt tacaggccca cccgacccgt gggcaggaat ttcactaacc 660
ttaggttctt acaacagatc aagagcagta gtcgccagga agcaccgaaa cccctggcct 720
tgccatctcg aggtacaaag aggcatctga gtctcaccag tacaagtgtg ccttcagcta 780
agaaggccag atgctatcct gtcccgagag tggaggaggg accccacagg caggtgctac 840
caaccccatc aggcaaatca tgggtgccaa gtcctgctcg gtcccccgag gtgcctactg 900
cagagaaaga tttgtcttct aaaggaaagg tg ctgacct gagtctctct gggtcggtgt 960
gctgtaaaca caagcccagc tccacatctc tgctgtcacc accccgccaa aatgcctttc 1020
agctcaggcc atttattgag accagacatt tcctttactc caggggagat ggccaagagc 1080
gtctaaaccc ctcattccta ctcagcaacc tccagcctaa cttgactggg gccaggagac 1140
tggtggagat catctttctg ggctcaaggc ctaggacatc aggaccactc tgcaggacac 1200
accgtctatc gcgtcgatac tggcagatgc ggcccctgtt ccaacagctg ctggtgaacc 1260
atgcagagtg ccaatatgtc agactcctca ggtcacattg caggtttcga acagcaaacc 1320
aacaggtgac agatgccttg aacaccagcc caccgcacct catggatttg ctccgcctgc 1380
acagcagtcc ctggcaggta tatggttttc ttcgggcctg tctctgcaag gtggtgtctg 1440
ctagtctctg gggtaccagg cacaatgagc gccgcttctt taagaactta aagaagttca 1500
tctcgttggg gaaatacggc aagctatcac tgcaggaact gatgtggaag atgaaagtag 1560
aggattgcca ctggctccgc agcagcccgg ggaaggaccg tgtccccgct gcagagcacc 1620
gtctgaggga gaggatcctg gctacgttcc tgctctggct gatggacaca tacgtggtac 1680
agctgcttag gtcattcttt tacatcacag agagcacatt ccagaagaac aggctcttct 1740
tctaccgtaa gagtgtgtgg agcaagctgc agagcattgg agtcaggcaa caccttgaga 1800
gagtgcggct acgggagctg tcacaagagg aggtcaggca tcaccaggac acctggctag 1860
ccatgcccat ctgcagactg cgcttcatcc ccaagcccaa cggcctgcgg cccattgtga 1920
acatgagtta tagcatgggt accagagctt tgggcagaag gaagcaggcc cagcatttca 1980
cccagcgtct caagactctc ttcagcatgc tcaactatga gcggacaaaa catcctcacc 2040
ttatggggtc ttctgtactg ggtatgaatg acatctacag gacctggcgg gcctttgtgc 2100
tgcgtgtgcg tgctctggac cagacaccca ggatgtactt tgttaaggca gatgtgaccg 2160
gggcctatga tgccatcccc cagggtaagc tggtggaggt tgttgccaat atgatcaggc 2220
actcggagag cacgtactgt atccgccagt atgcagtggt ccggagagat agccaaggcc 2280
aagtccacaa gtcctttagg agacaggtca ccaccctctc tgacctccag ccatacatgg 2340
gccagttcct taagcatctg caggattcag atgccagtgc actgaggaac tccgttgtca 2400
tcgagcagag catctctatg aatgagagca gcagcagcct gtttgacttc ttcctgcact 2460
tcctgcgtca cagtgtcgta aagattggtg acaggtgcta tacgcagtgc cagggcatcc 2520
cccagggctc cagcctatcc accctgctct gcagtctgtg tttcggagac atggagaaca 2580
agctgtttgc tgaggtgcag cgggatgggt tgcttttacg ttttgttgat gactttctgt 2640
tggtgacgcc tcacttggac caagcaaaaa ccttcctcag caccctggtc catggcgttc 2700
ctgagtatgg gtgcatgata aacttgcaga agacagtggt gaacttccct gtggagcctg 2760
gtaccctggg tggtgcagct ccataccagc tgcctgctca ctgcctgttt ccctggtgtg 2820
gcttgctgct ggacactcag actttggagg tg tctgtga ctactcaggt tatgcccaga 2880
cctcaattaa gacgagcctc accttccaga gtgtcttcaa agctgggaag accatgcgga 2940
acaagctcct gtcggtcttg cggttgaagt gtcacggtct atttctagac ttgcaggtga 3000
acagcctcca gacagtctgc atcaatatat acaagatctt cctgcttcag gcctacaggt 3060
tccatgcatg tgtgattcag cttccctttg accagcgtgt taggaagaac ctcacattct 3120
ttctgggcat catctccagc caagcatcct gctgctatgc tatcctgaag gtcaagaatc 3180
caggaatgac actaaaggcc tctggctcct ttcctcctga agccgcacat tggctctgct 3240
accaggcctt cctgctcaag ctggctgctc attctgtcat ctacaaatgt ctcctgggac 3300
ctctgaggac agcccaaaaa ctgctgtgcc ggaagctccc agaggcgaca atgaccatcc 3360
ttaaagctgc agctgaccca gccctaagca cagactttca gaccattttg gactaataat 3420
taagctgcct tctgcggggc ttgccttctg gccatgccct tcttctctcc cttgcacctg 3480
tacctcttgg tctttgaata aagcctgagt aggaagtcta gaaaaaaaaa aaaaaaaaaa 3540
aaaaaaaaaa aaaaaaaaaa aaaaaaaaaa aaaaaaaaaa aaaaaaaaaa aaaaaaaaaa 3600
a 3601
SEQ ID NO: 3
aggaaataag agagaaaaga agagtaagaa gaaatataag agccacc 47
SEQ ID NO: 4
atgacccgcg ctcctcgttg ccccgcggtg cgctctctgc tgcgcagccg ataccgggag 60
gtgtggccgc tggcaacctt tgtgcggcgc ctggggcccg agggcaggcg gcttgtgcaa 120
cccggggacc cgaagatcta ccgcactttg gttgcccaat gcctagtgtg catgcactgg 180
ggctcacagc ctccacctgc cgacctttcc ttccaccagg tgtcatccct gaaagagctg 240
gtggccaggg ttgtgcagag actctgcgag cgcaacgaga gaaacgtgct ggcttttggc 300
tttgagctgc ttaacgaggc cagaggcggg ccccccatgg ccttcactag tagcgtgcgt 360
agctacttgc ccaacactgt tattgagacc ctgcgtgtca gtggtgcatg gatgctactg 420
ttgagccgag tgggcgacga cctgctggtc tacctgctgg cacactgtgc tctttatctt 480
ctggtgcccc ccagctgtgc ctaccaggtg tgugggtctc ccctgtacca aatttgtgcc 540
accacggata tctggccctc tgtgtccgct agttacaggc ccacccgacc cgtgggcagg 600
aatttcacta accttaggtt cttacaacag atcaagagca gtagtcgcca ggaagcaccg 660
aaacccctgg ccttgccatc tcgaggtaca aagaggcatc tgagtctcac cagtacaagt 720
gtgccttcag ctaagaaggc cagatgctat cctgtcccga gagtggagga gggaccccac 780
aggcaggtgc taccaacccc atcaggcaaa tcatgggtgc caagtcctgc tcggtccccc 840
gaggtgccta ctgcagagaa agatttgtct tctaaaggaa aggtgtctga cctgagtctc 900
tctgggtcgg tgtgctgtaa acacaagccc agctccacat ctctgctgtc accaccccgc 960
caaaatgcct ttcagctcag gccatttatt gagaccagac atttccttta ctccagggga 1020
gatggccaag agcgtctaaa cccctcattc ctactcagca acctccagcc taacttgact 1080
ggggccagga gactggtgga gatcatcttt ctgggctcaa ggcctaggac atcaggacca 1140
ctctgcagga cacaccgtct atcgcgtcga tactggcaga tgcggcccct gttccaacag 1200
ctgctggtga accatgcaga gtgccaatat gtcagactcc tcaggtcaca ttgcaggttt 1260
cgaacagcaa accaacaggt gacagatgcc ttgaacacca gcccaccgca cctcatggat 1320
ttgctccgcc tgcacagcag tccctggcag gtatatggtt ttcttcgggc ctgtctctgc 1380
aaggtggtgt ctgctagtct ctggggtacc aggcacaatg agcgccgctt ctttaagaac 1440
ttaaagaagt tcatctcgtt ggggaaatac ggcaagctat cactgcagga actgatgtgg 1500
aagatgaaag tagaggattg ccactggctc cgcagcagcc cggggaagga ccgtgtcccc 1560
gctgcagagc accgtctgag ggagaggatc ctggctacgt tcctgttctg gctgatggac 1620
acatacgtgg tacagctgct taggtcattc ttttacatca cagagagcac attccagaag 1680
aacaggctct tcttctaccg taagagtgtg tggagcaagc tgcagagcat tggagtcagg 1740
caacaccttg agagagtgcg gctacgggag ctgtcacaag aggaggtcag gcatcaccag 1800
gacacctggc tagccatgcc catctgcaga ctgcgcttca tccccaagcc caacggcctg 1860
cggcccattg tgaacatgag ttatagcatg ggtaccagag ctttgggcag aaggaagcag 1920
gcccagcatt tcacccagcg tctcaagact ctcttcagca tgctcaacta tgagcggaca 1980
aaacatcctc accttatggg gtcttctgta ctgggtatga atgacatcta caggacctgg 2040
cgggcctttg tgctgcgtgt gcgtgctctg gaccagacac ccaggatgta ctttgttaag 2100
gcagatgtga ccggggccta tgatgccatc ccccagggta agctggtgga ggttgttgcc 2160
aatatgatca ggcactcgga gagcacgtac tgtatccgcc agtatgcagt ggtccggaga 2220
gatagccaag gccaagtcca caagtccttt aggagacagg tcaccaccct ctctgacctc 2280
cagccataca tgggccagtt ccttaagcat ctgcaggatt cagatgccag tgcactgagg 2340
aactccgttg tcatcgagca gagcatctct atgaatgaga gcagcagcag cctgtttgac 2400
ttcttcctgc acttcctgcg tcacagtgtc gtaaagattg gtgacaggtg ctatacgcag 2460
tgccagggca tcccccaggg ctccagccta tccaccctgc tctgcagtct gtgtttcgga 2520
gacatggaga acaagctgtt tgctgaggtg cagcgggatg ggttgctttt acgttttgtt 2580
gatgactttc tgttggtgac gcctcacttg gaccaagcaa aaaccttcct cagcaccctg 2640
gtccatggcg ttcctgagta tgggtgcatg ataaacttgc agaagacagt ggtgaacttc 2700
cctgtggagc ctggtaccct gggtggtgca gctccatacc agctgcctgc tcactgcctg 2760
tttccctggt gtggcttgct gctggacact cagactttgg aggtgttctg tgactactca 2820
ggttatgccc agacctcaat taagacgagc ctcaccttcc agagtgtctt caaagctggg 2880
aagaccatgc ggaacaagct cctgtcggtc ttgcggttga agtgtcacgg tctatttcta 2940
gactcgcagg tgaacagcct ccagacagtc tgcatcaata tatacaagat cttcctgctt 3000
caggcctaca ggttccatgc atgtgtgatt cagcttccct ttgaccagcg tgttaggaag 3060
aacctcacat tctttctggg catcatctcc agccaagcat cctgctgcta tgctatcctg 3120
aaggtcaaga atccaggaat gacactaaag gcctctggct cctttcctcc tgaagccgca 3180
cattggctct gctaccaggc cttcctgctc aagctggctg ctcattctgt catctacaaa 3240
tgtctcctgg gacctctgag gacagcccaa aaactgctgt gccggaagct cccagaggcg 3300
acaatgacca tccttaaagc tgcagctgac ccagccctaa gcacagactt tcagaccatt 3360
ttggactaa 3369
SEQ ID NO: 5
ttaagctgcc ttctgcgggg cttgccttct ggccatgccc ttcttctctc ccttgcacct 60
gtacctcttg gtctttgaat aaagcctgag taggaagtct ag 102

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

Citation of the above publications or documents is not intended as an admission that any of the foregoing is pertinent prior art, nor does it constitute any admission as to the contents or date of these publications or documents.

1. A method of delivering a polynucleotide to a subject, comprising administering, by intravenous injection, a polynucleotide encapsulated in a lipid nanoparticle (LNP) comprising a cationic lipid in a molar percentage of between about 25% and about 60%, and either:
(a) an ionizable lipid comprising SS-OP, or an SS-OP analog, at a molar percentage of between about 25% and about 65%, wherein the LNP has a cationic lipid:ionizable lipid molar (C/I) ratio of between 0.7 and 1.8, and a lipid nitrogen:polynucleotide phosphate (N/P) ratio of between 10.5 and 31; or,
(b) an ionizable lipid comprising DLin-MC3-DMA, or a DLin-MC3-DMA analog, at a molar percentage of between at or about 30% and at or about 50%, wherein the LNP has a C/I ratio of between 0.5 and 2.5, and an N/P ratio of between 2.5 and 8.

2. The method of claim 1, wherein the polynucleotide comprises a synthetic RNA, which upon or after administration of the LNP, the mRNA is translated into a corresponding protein encoded by the mRNA in vivo in the subject, and the half-life of the lipid DOTAP in vivo in the:
lung of the subject is at least 30 hours;
a) liver of the subject is no more than 11 hours; and/or
b) spleen of the subject is no more than 9 hours.

3. The method of claim 2, wherein the half-life of the protein in vivo in the lung of the subject is at least 10 hours, 15 hours, 20 hours, 25 hours, 30 hours, or 35 hours.

4. The method of claim 1, wherein the polynucleotide comprises a synthetic RNA, which upon or after administration of the LNP, the mRNA is translated into a corresponding protein encoded by the mRNA in vivo in the subject, and wherein at or about 6 hours after intravenous infusion of the LNP, the lung:liver ratio of the amount of the protein per mg of tissue is at least 2:1, 3:1, or 4:1.

5. The method of claim 4, wherein the lung:liver ratio of the amount of the protein per mg of tissue is at least 5:1, 6:1, 7:1, 8:1, 9:1, 10:1, 20:1, or 50:1.

6. The method of claim 1, wherein the polynucleotide comprises a synthetic RNA encoding a telomerase reverse transcriptase (TERT), which upon or after administration of the LNP, the mRNA is translated into a corresponding TERT protein in vivo in the subject.

7. The method of claim 6, wherein the median telomere length of lung alveolar cells is extended by at least 5%, at least 10%, at least 20%, or at least 50%.

8. The method of claim 6, wherein the median telomere length of lung AT2 cells is extended by at least 5%, at least 10%, at least 20%, or at least 50%.

9. The method of claim 6, wherein the increase in telomerase activity in the lung is detectable by the TRAP assay for at least 24 or 48 hours.

10. The method of claim 1, wherein the ionizable lipid comprises SS-OP, or an SS-OP analog, and the LNP has an N/P ratio of: (a) between 28 and 31 or (b) at or about 29.

11. (canceled)

12. The method of claim 1, wherein the ionizable lipid comprises SS-OP, or an SS-OP analog, and the LNP has an N/P ratio of: (a) between 10.5 and 15, (b) at or about 12, or (c) at or about 12.1.

13. (canceled)

14. The method of claim 1, wherein the ionizable lipid comprises DLin-MC3-DMA, or a DLin-MC3-DMA analog, and the LNP has an N/P ratio of (a) between 3 and 5, or (b) at or about 4.5.

15. (canceled)

16. The method of claim 1, wherein the ionizable lipid is SS-OP and the LNP has a C/I ratio at or about 1.8 and is composed of 5 different types of lipids.

17. The method of claim 1, wherein the ionizable lipid is SS-OP and the LNP has a C/I ratio at or about 1.8 and is composed of 5 lipids, including:
(a) DOTAP, DOPC and three additional different types of lipids,
(b) DMC-PRO2000, DOPC, DOTAP and two additional different types of lipids
wherein DOTAP is the cationic lipid.

18. (canceled)

19. The method of claim 1, wherein the ionizable lipid is SS-OP and the LNP has a C/I ratio at or about 0.8 and is composed of 3 or 5 different types of lipids.

20. The method of claim 1, wherein the ionizable lipid is SS-OP and the LNP has a C/I ratio at or about 0.8 and is composed of 3 or 5 different types of lipids, including:
(a) the ionizable lipid, DOTAP, and one or optionally three additional different types of lipids, or
(b) the ionizable lipid, DMG-PBG2000, DOTAP and one or optionally three additional different types of lipids;
wherein DOTAP is the cationic lipid.

21. (canceled)

22. The method of claim 1, wherein the ionizable lipid is DLin-MC3-DMA and the LNP has a C/I ratio at or about 1.5 and is composed of 3 lipids.

23. The method of claim 1, wherein the ionizable lipid is DLin-MC3-DMA and the LNP has a C/I ratio at or about 1.5 and is composed of 3 lipids, including:
(a) the ionizable lipid, DOTAP, and another different type of lipid,
(b) the ionizable lipid, DMC-PEG2000 and DOTAP, or
(c) the ionizable lipid, DMG-PEG2000 and DOTAP, and N/P ratio is about 4.5;
wherein DOTAP is the cationic lipid.

24. (canceled)

25. (canceled)

26. The method of claim 1, wherein the LNP is selected from an LNP of Tables 8-10.


SEQUENCE LISTING (5 sequences)

SEQ ID NO: 1 - DNA, 3399 bp, synthetic construct
  source 1..3399: mol_type = other DNA
atgcccagagcccccagatgcagagccgtgagaagcctgctgagaagccactacagagaggtgctgcccctggccaccttcgtgagaagactgggcccccagggctggagactggtgcagagaggcgaccccgccgccttcagagccctggtggcccagtgcctggtgtgcgtgccctgggacgccagaccccctcccgccgcccccagcttcagacaggtgagctgcctgaaggagctggtggccagagtgctgcagagactgtgcgagagaggcgccaagaacgtgctggccttcggcttcgccctgctggacggcgccagaggcggccctcccgaggccttcaccaccagcgtgagaagctacctgcccaacaccgtgaccgacgccctgagaggcagcggcgcctggggcctgctgctgagaagagtgggcgacgacgtgctggtgcacctgctggccagatgcgccctgttcgtgctggtggcccccagctgcgcctaccaggtgtgcggccctcccctgtaccagctgggcgccgccacccaggccagaccccctccccacgccagcggccccagaagaagactgggctgcgagagagcctggaaccacagcgtgagagaggccggcgtgcccctgggcctgcccgcccccggcgccagaagaagaggcggcagcgccagcagaagcctgcccctgcccaagagacccagaagaggcgccgcccccgagcccgagagaacccccgtgggccagggcagctgggcccaccccggcagaaccagaggccccagcgacagaggcttctgcgtggtgagccccgccagacccgccgaggaggccaccagcctggagggcgccctgagcggcaccagacacagccaccccagcgtgggcagacagcaccacgccggccctcccagcaccagcagacctcccagaccctgggacaccccctgccctcccgtgtacgccgagaccaagcacttcctgtacagcagcggcgacaaggagcagctgagacccagcttcctgctgagcagcctgagacccagcctgaccggcgccagaagactggtggagaccatcttcctgggcagcagaccctggatgcccggcacccccagaagactgcccagactgccccagagatactggcagatgagacccctgttcctggagctgctgggcaaccacgcccagtgcccctacggcgtgctgctgaagacccactgccccctgagagccgccgtgacccccgccgccggcgtgtgcgccagagagaagccccagggcagcgtggccgcccccgaggaggaggacaccgaccccagaagactggtgcagctgctgagacagcacagcagcccctggcaggtgtacggcttcgtgagagcctgcctgagaagactggtgcctcccggcctgtggggcagcagacacaacgagagaagattcctgagaaacaccaagaagttcatcagcctgggcaagcacgccaagctgagcctgcaggagctgacctggaagatgagcgtgagagactgcgcctggctgagaagaagccccggcgtgggctgcgtgcccgccgccgagcacagactgagagaggagatcctggccaagttcctgcactggctgatgagcgtgtacgtggtggagctgctgagaagcttcttctacgtgaccgagaccaccttccagaagaacagactgttcttctacagaaagagcgtgtggagcaagctgcagagcatcggcatcagacagcacctgaagagagtgcagctgagagagctgagcgaggccgaggtgagacagcacagagaggccagacccgccctgctgaccagcagactgagattcatccccaagcccgacggcctgagacccatcgtgaacatggactacgtggtgggcgccagaaccttcagaagagagaagagagccgagagactgaccagcagagtgaaggccctgttcagcgtgctgaactacgagagagccagaagacccggcctgctgggcgccagcgtgctgggcctggacgacatccacagagcctggagaaccttcgtgctgagagtgagagcccaggatccccctcccgagctgtacttcgtgaaggtggacgtgaccggcgcctacgacaccatcccccaggacagactgaccgaggtgatcgccagcatcatcaagccccagaacacctactgcgtgagaagatacgccgtggtgcagaaggccgcccacggccacgtgagaaaggccttcaagagccacgtgagcaccctgaccgacctgcagccctacatgagacagttcgtggcccacctgcaggagaccagccccctgagagacgccgtggtgatcgagcagagcagcagcctgaacgaggccagcagcggcctgttcgacgtgttcctgagattcatgtgccaccacgccgtgagaatcagaggcaagagctacgtgcagtgccagggcatcccccagggcagcatcctgagcaccctgctgtgcagcctgtgctacggcgacatggagaacaagctgttcgccggcatcagaagagacggcctgctgctgagactggtggacgacttcctgctggtgacaccccacctgacccacgccaagaccttcctgagaaccctggtgagaggcgtgcccgagtacggctgcgtggtgaacctgagaaagaccgtggtgaacttccccgtggaggacgaggccctgggcggcaccgccttcgtgcagatgcccgcccacggcctgttcccctggtgcggcctgctgctggacaccagaaccctggaggtgcagagcgactacagcagctacgccagaaccagcatcagagccagcctgaccttcaacagaggcttcaaggccggcagaaacatgagaagaaagctgttcggcgtgctgagactgaagtgccacagcctgttcctggacctgcaggtgaacagcctgcagaccgtgtgcaccaacatctacaagatcctgctgctgcaggcctacagattccacgcctgcgtgctgcagctgcccttccaccagcaggtgtggaagaaccccaccttcttcctgagagtgatcagcgacaccgccagcctgtgctacagcatcctgaaggccaagaacgccggcatgagcctgggcgccaagggcgccgccggccccctgcccagcgaggccgtgcagtggctgtgccaccaggccttcctgctgaagctgaccagacacagagtgacctacgtgcccctgctgggcagcctgagaaccgcccagacccagctgagcagaaagctgcccggcaccaccctgaccgccctggaggccgccgccaaccccgccctgcccagcgacttcaagaccatcctggactga

SEQ ID NO: 2 - DNA, 3601 bp, Mus muculus
  source 1..3601: mol_type = genomic DNA
aggaaataagagagaaaagaagagtaagaagaaatataagagccaccatgacccgcgctcctcgttgccccgcggtgcgctctctgctgcgcagccgataccgggaggtgtggccgctggcaacctttgtgcggcgcctggggcccgagggcaggcggcttgtgcaacccggggacccgaagatctaccgcactttggttgcccaatgcctagtgtgcatgcactggggctcacagcctccacctgccgacctttccttccaccaggtgtcatccctgaaagagctggtggccagggttgtgcagagactctgcgagcgcaacgagagaaacgtgctggcttttggctttgagctgcttaacgaggccagaggcgggcctcccatggccttcactagtagcgtgcgtagctacttgcccaacactgttattgagaccctgcgtgtcagtggtgcatggatgctactgttgagccgagtgggcgacgacctgctggtctacctgctggcacactgtgctctttatcttctggtgccccccagctgtgcctaccaggtgtgtgggtctcccctgtaccaaatttgtgccaccacggatatctggccctctgtgtccgctagttacaggcccacccgacccgtgggcaggaatttcactaaccttaggttcttacaacagatcaagagcagtagtcgccaggaagcaccgaaacccctggccttgccatctcgaggtacaaagaggcatctgagtctcaccagtacaagtgtgccttcagctaagaaggccagatgctatcctgtcccgagagtggaggagggaccccacaggcaggtgctaccaaccccatcaggcaaatcatgggtgccaagtcctgctcggtcccccgaggtgcctactgcagagaaagatttgtcttctaaaggaaaggtgtctgacctgagtctctctgggtcggtgtgctgtaaacacaagcccagctccacatctctgctgtcaccaccccgccaaaatgcctttcagctcaggccatttattgagaccagacatttcctttactccaggggagatggccaagagcgtctaaacccctcattcctactcagcaacctccagcctaacttgactggggccaggagactggtggagatcatctttctgggctcaaggcctaggacatcaggaccactctgcaggacacaccgtctatcgcgtcgatactggcagatgcggcccctgttccaacagctgctggtgaaccatgcagagtgccaatatgtcagactcctcaggtcacattgcaggtttcgaacagcaaaccaacaggtgacagatgccttgaacaccagcccaccgcacctcatggatttgctccgcctgcacagcagtccctggcaggtatatggttttcttcgggcctgtctctgcaaggtggtgtctgctagtctctggggtaccaggcacaatgagcgccgcttctttaagaacttaaagaagttcatctcgttggggaaatacggcaagctatcactgcaggaactgatgtggaagatgaaagtagaggattgccactggctccgcagcagcccggggaaggaccgtgtccccgctgcagagcaccgtctgagggagaggatcctggctacgttcctgttctggctgatggacacatacgtggtacagctgcttaggtcattcttttacatcacagagagcacattccagaagaacaggctcttcttctaccgtaagagtgtgtggagcaagctgcagagcattggagtcaggcaacaccttgagagagtgcggctacgggagctgtcacaagaggaggtcaggcatcaccaggacacctggctagccatgcccatctgcagactgcgcttcatccccaagcccaacggcctgcggcccattgtgaacatgagttatagcatgggtaccagagctttgggcagaaggaagcaggcccagcatttcacccagcgtctcaagactctcttcagcatgctcaactatgagcggacaaaacatcctcaccttatggggtcttctgtactgggtatgaatgacatctacaggacctggcgggcctttgtgctgcgtgtgcgtgctctggaccagacacccaggatgtactttgttaaggcagatgtgaccggggcctatgatgccatcccccagggtaagctggtggaggttgttgccaatatgatcaggcactcggagagcacgtactgtatccgccagtatgcagtggtccggagagatagccaaggccaagtccacaagtcctttaggagacaggtcaccaccctctctgacctccagccatacatgggccagttccttaagcatctgcaggattcagatgccagtgcactgaggaactccgttgtcatcgagcagagcatctctatgaatgagagcagcagcagcctgtttgacttcttcctgcacttcctgcgtcacagtgtcgtaaagattggtgacaggtgctatacgcagtgccagggcatcccccagggctccagcctatccaccctgctctgcagtctgtgtttcggagacatggagaacaagctgtttgctgaggtgcagcgggatgggttgcttttacgttttgttgatgactttctgttggtgacgcctcacttggaccaagcaaaaaccttcctcagcaccctggtccatggcgttcctgagtatgggtgcatgataaacttgcagaagacagtggtgaacttccctgtggagcctggtaccctgggtggtgcagctccataccagctgcctgctcactgcctgtttccctggtgtggcttgctgctggacactcagactttggaggtgttctgtgactactcaggttatgcccagacctcaattaagacgagcctcaccttccagagtgtcttcaaagctgggaagaccatgcggaacaagctcctgtcggtcttgcggttgaagtgtcacggtctatttctagacttgcaggtgaacagcctccagacagtctgcatcaatatatacaagatcttcctgcttcaggcctacaggttccatgcatgtgtgattcagcttccctttgaccagcgtgttaggaagaacctcacattctttctgggcatcatctccagccaagcatcctgctgctatgctatcctgaaggtcaagaatccaggaatgacactaaaggcctctggctcctttcctcctgaagccgcacattggctctgctaccaggccttcctgctcaagctggctgctcattctgtcatctacaaatgtctcctgggacctctgaggacagcccaaaaactgctgtgccggaagctcccagaggcgacaatgaccatccttaaagctgcagctgacccagccctaagcacagactttcagaccattttggactaataattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 3 - DNA, 47 bp, synthetic construct
  source 1..47: mol_type = other DNA
aggaaataagagagaaaagaagagtaagaagaaatataagagccacc

SEQ ID NO: 4 - DNA, 3369 bp, Mus muculus
  source 1..3369: mol_type = genomic DNA
atgacccgcgctcctcgttgccccgcggtgcgctctctgctgcgcagccgataccgggaggtgtggccgctggcaacctttgtgcggcgcctggggcccgagggcaggcggcttgtgcaacccggggacccgaagatctaccgcactttggttgcccaatgcctagtgtgcatgcactggggctcacagcctccacctgccgacctttccttccaccaggtgtcatccctgaaagagctggtggccagggttgtgcagagactctgcgagcgcaacgagagaaacgtgctggcttttggctttgagctgcttaacgaggccagaggcgggcctcccatggccttcactagtagcgtgcgtagctacttgcccaacactgttattgagaccctgcgtgtcagtggtgcatggatgctactgttgagccgagtgggcgacgacctgctggtctacctgctggcacactgtgctctttatcttctggtgccccccagctgtgcctaccaggtgtgtgggtctcccctgtaccaaatttgtgccaccacggatatctggccctctgtgtccgctagttacaggcccacccgacccgtgggcaggaatttcactaaccttaggttcttacaacagatcaagagcagtagtcgccaggaagcaccgaaacccctggccttgccatctcgaggtacaaagaggcatctgagtctcaccagtacaagtgtgccttcagctaagaaggccagatgctatcctgtcccgagagtggaggagggaccccacaggcaggtgctaccaaccccatcaggcaaatcatgggtgccaagtcctgctcggtcccccgaggtgcctactgcagagaaagatttgtcttctaaaggaaaggtgtctgacctgagtctctctgggtcggtgtgctgtaaacacaagcccagctccacatctctgctgtcaccaccccgccaaaatgcctttcagctcaggccatttattgagaccagacatttcctttactccaggggagatggccaagagcgtctaaacccctcattcctactcagcaacctccagcctaacttgactggggccaggagactggtggagatcatctttctgggctcaaggcctaggacatcaggaccactctgcaggacacaccgtctatcgcgtcgatactggcagatgcggcccctgttccaacagctgctggtgaaccatgcagagtgccaatatgtcagactcctcaggtcacattgcaggtttcgaacagcaaaccaacaggtgacagatgccttgaacaccagcccaccgcacctcatggatttgctccgcctgcacagcagtccctggcaggtatatggttttcttcgggcctgtctctgcaaggtggtgtctgctagtctctggggtaccaggcacaatgagcgccgcttctttaagaacttaaagaagttcatctcgttggggaaatacggcaagctatcactgcaggaactgatgtggaagatgaaagtagaggattgccactggctccgcagcagcccggggaaggaccgtgtccccgctgcagagcaccgtctgagggagaggatcctggctacgttcctgttctggctgatggacacatacgtggtacagctgcttaggtcattcttttacatcacagagagcacattccagaagaacaggctcttcttctaccgtaagagtgtgtggagcaagctgcagagcattggagtcaggcaacaccttgagagagtgcggctacgggagctgtcacaagaggaggtcaggcatcaccaggacacctggctagccatgcccatctgcagactgcgcttcatccccaagcccaacggcctgcggcccattgtgaacatgagttatagcatgggtaccagagctttgggcagaaggaagcaggcccagcatttcacccagcgtctcaagactctcttcagcatgctcaactatgagcggacaaaacatcctcaccttatggggtcttctgtactgggtatgaatgacatctacaggacctggcgggcctttgtgctgcgtgtgcgtgctctggaccagacacccaggatgtactttgttaaggcagatgtgaccggggcctatgatgccatcccccagggtaagctggtggaggttgttgccaatatgatcaggcactcggagagcacgtactgtatccgccagtatgcagtggtccggagagatagccaaggccaagtccacaagtcctttaggagacaggtcaccaccctctctgacctccagccatacatgggccagttccttaagcatctgcaggattcagatgccagtgcactgaggaactccgttgtcatcgagcagagcatctctatgaatgagagcagcagcagcctgtttgacttcttcctgcacttcctgcgtcacagtgtcgtaaagattggtgacaggtgctatacgcagtgccagggcatcccccagggctccagcctatccaccctgctctgcagtctgtgtttcggagacatggagaacaagctgtttgctgaggtgcagcgggatgggttgcttttacgttttgttgatgactttctgttggtgacgcctcacttggaccaagcaaaaaccttcctcagcaccctggtccatggcgttcctgagtatgggtgcatgataaacttgcagaagacagtggtgaacttccctgtggagcctggtaccctgggtggtgcagctccataccagctgcctgctcactgcctgtttccctggtgtggcttgctgctggacactcagactttggaggtgttctgtgactactcaggttatgcccagacctcaattaagacgagcctcaccttccagagtgtcttcaaagctgggaagaccatgcggaacaagctcctgtcggtcttgcggttgaagtgtcacggtctatttctagacttgcaggtgaacagcctccagacagtctgcatcaatatatacaagatcttcctgcttcaggcctacaggttccatgcatgtgtgattcagcttccctttgaccagcgtgttaggaagaacctcacattctttctgggcatcatctccagccaagcatcctgctgctatgctatcctgaaggtcaagaatccaggaatgacactaaaggcctctggctcctttcctcctgaagccgcacattggctctgctaccaggccttcctgctcaagctggctgctcattctgtcatctacaaatgtctcctgggacctctgaggacagcccaaaaactgctgtgccggaagctcccagaggcgacaatgaccatccttaaagctgcagctgacccagccctaagcacagactttcagaccattttggactaa

SEQ ID NO: 5 - DNA, 102 bp, synthetic construct
  source 1..102: mol_type = other DNA
ttaagctgccttctgcggggcttgccttctggccatgcccttcttctctcccttgcacctgtacctcttggtctttgaataaagcctgagtaggaagtctag